SEQUENCE LISTING (19 sequences)

SEQ ID NO: 1 - DNA, 366 bp, Artificial
  Synthetic sequence F45D9 VH encoding nucleotide sequence
caggtgcagctgcagcagtggggagccggcctgctgaagccctctgagacactgagcctgatctgcgctgtgtacggcggatctttcagcacctactactggacctggattaggcagcccccaggcaagggcctggaatggatcggcgagatcaaccacaggggcaccaccaactacagccccagcctgaagagcagagtgaccatcagcgtggacaccagcaagaaccacatcagcctgaacctgaccagcgtgacagccgccgacaccgccctgtactactgcgcccgcggactgctgtggattggggagggcgactacggcctggacgtgtggggacagggcaccaccgtgaccgtgtccagc

SEQ ID NO: 2 - protein, 122 aa, Artificial
  Synthetic sequence F45D9 VH amino acid sequence
QVQLQQWGAGLLKPSETLSLICAVYGGSFSTYYWTWIRQPPGKGLEWIGEINHRGTTNYSPSLKSRVTISVDTSKNHISLNLTSVTAADTALYYCARGLLWIGEGDYGLDVWGQGTTVTVSS

SEQ ID NO: 3 - DNA, 324 bp, Artificial
  Synthetic sequence F45D9 VL encoding nucleotide sequence
gacatccagatgacccagagcccctctagcctcagcgctagcgtcggcgacagagtgaccatcacctgcagggccagccagggcatcaggcggtggctggcctggtatcagcagaagcccgagaaggcccccaagagcctgatctacgccgccagctccctgcagagcggcgtgcccagcaggttcagcggcagcggctccggcaccgacttcaccctgaccatcagcagcctgcagcccgaggacttcgccacctactactgccagcagtacaacagctacccctacaccttcggccagggcaccaagctggaaatcaagagg

SEQ ID NO: 4 - protein, 108 aa, Artificial
  Synthetic sequence F45D9 VL amino acid sequence
DIQMTQSPSSLSASVGDRVTITCRASQGIRRWLAWYQQKPEKAPKSLIYAASSLQSGVPSRFSGSGSGTDFTLTISSLQPEDFATYYCQQYNSYPYTFGQGTKLEIKR

SEQ ID NO: 5 - protein, 6 aa, Artificial
  Synthetic sequence F45D9 VH CDR1 amino acid sequence
TYYWTW

SEQ ID NO: 6 - protein, 16 aa, Artificial
  Synthetic sequence F45D9 VH CDR2 amino acid sequence
EINHRGTTNYSPSLKS

SEQ ID NO: 7 - protein, 14 aa, Artificial
  Synthetic sequence F45D9 VH CDR3 amino acid sequence
GLLWIGEGDYGLDV

SEQ ID NO: 8 - protein, 11 aa, Artificial
  Synthetic sequence F45D9 VL CDR1 amino acid sequence
RASQGIRRWLA

SEQ ID NO: 9 - protein, 7 aa, Artificial
  Synthetic sequence F45D9 VL CDR2 amino acid sequence
AASSLQS

SEQ ID NO: 10 - protein, 9 aa, Artificial
  Synthetic sequence F45D9 VL CDR3 amino acid sequence
QQYNSYPYT

SEQ ID NO: 11 - protein, 319 aa, Homo sapiens
RLSSKSVNAQVTDINSKGLELRKTVTTVETQNLEGLHHDGQFCHKPCPPGERKARDCTVNGDEPDCVPCQEGKEYTDKAHFSSKCRRCRLCDEGHGLEVEINCTRTQNTKCRCKPNFFCNSTVCEHCDPCTKCEHGIIKECTLTSNTKCKEEGSRSNLGWLCLLLLPIPLIVWVKRKEVQKTCRKHRKENQGSHESPTLNPETVAINLSDVDLSKYITTIAGVMTLSQVKGFVRKNGVNEAKIDEIKNDNVQDTAEQKVQLLRNWHQLHGKKEAYDTLIKDLKKANLCTLAEKIQTIILKDITSDSENSNFRNEIQSLV

SEQ ID NO: 12 - protein, 30 aa, Homo sapiens
HGIIKECTLTSNTKCKEEGSRSNLGWLCLL

SEQ ID NO: 13 - protein, 30 aa, Homo sapiens
ECTLTSNTKCKEEGSRSNLGWLCLLLLPIP

SEQ ID NO: 14 - protein, 30 aa, Homo sapiens
SNTKCKEEGSRSNLGWLCLLLLPIPLIVWV

SEQ ID NO: 15 - protein, 20 aa, Homo sapiens
SNTKCKEEGSRSNLGWLCLL

SEQ ID NO: 16 - protein, 468 aa, Artificial [sequence omitted]

SEQ ID NO: 17 - protein, 40 aa, Homo sapiens
HGIIKECTLTSNTKCKEEGSRSNLGWLCLLLLPIPLIVWV

SEQ ID NO: 18 - protein, 4 aa, Artificial sequence
  Synthetic sequence IgG1 like hinge sequence
CPPC

SEQ ID NO: 19 - protein, 4 aa, Homo sapiens
CPSC

(19) United States
(12) Patent Application Publication
(10) Pub. No.: US 2010/0233157 A1
(43) Pub. Date: Sep. 16, 2010

(54) HUMAN ANTIBODIES AGAINST HUMAN FAS AND THEIR USE

(76) Inventors: LYDA M. OSORIO, STOCKHOLM (SE); MAORONG RUAN, MALMO (SE); FRANCESCA CHIODI, STOCKHOLM (SE); HANS-PETER EKRE, STOCKHOLM (SE)

Correspondence Address: KLARQUIST SPARKMAN, LLP, 121 SW SALMON STREET, SUITE 1600, PORTLAND, OR, 97204, US

(21) Appl. No.: 12/721,482

(22) Filed: Mar. 10, 2010

Related U.S. Application Data
(60) Provisional application No. 61/159,762, filed on Mar. 12, 2009.

(51) Int. Cl. A61K 39/395; C07K 16/18; C07H 21/04; C12N 1/21; C12N 5/10; C12N 1/19; C12N 7/01; C12P 21/00; A61P 31/12; A61P 3/00; A61P 25/00

(52) U.S. Cl. 424/130.1; 530/387.1; 530/387.3; 536/23.53; 435/252.3; 435/325; 435/254.2; 435/235.1; 435/358; 435/367; 435/354; 435/69.6

(57) ABSTRACT

Binding members directed to human Fas (Fas), in particular antibody molecules against human Fas, employing the antibody VH and/or VL domain of the antibody molecule termed F45D9, which may be in IgG1 or IgG4 format. Methods of use in patients, diseases or disorders involving apoptosis, such as Graft-Versus-Host Disease, HIV-infection, Stevens-Johnson syndrome or Toxic epidermal necrolysis, Islet transplantation as treatment for insulin-dependent diabetes, diseases based on ischemia or ischemic reperfusion injury, heart disease, renal disease, neurological disorders and injuries and lymphocyte depletion in cancer patients associated to cytotoxic antineoplastic therapy.

The present invention relates to binding members directed to human Fas (Fas), in particular antibody molecules against human Fas. Preferred embodiments of the present invention employ the antibody VH and/or VL domain of the antibody molecule herein termed F45D9. Further preferred embodiments employ one or more complementarity determining regions (CDRs) of the F45D9 heavy chain variable (VH) and/or light chain variable (VL) domains, especially VH CDR3 in other antibody framework regions.

Fas/APO-1 first appeared in the literature in 1989, when it was described by two independent groups led by Minako Yonehara in Japan and Peter Krammer in Germany (Yonehara, S. et al., J. Exp. Med., 169:1747-1756, 1989) (Trauth B. C. et al., Science, 245:301-305, 1989). Both teams reported that Fas (CD95) was a cell surface molecule, expressed on human lymphocytes, which triggered cell death when cross-linked with agonistic anti-Fas antibodies.

Apoptosis mediated by interaction of CD95 and its ligand CD95L is one of the best understood apoptotic system in T cells. CD95 is a 45-kDa type I transmembrane protein and belongs to the tumor necrosis factor (TNF) receptor family (Itoh, N. et al. Cell, 66:233-243, 1991). CD95 is widely expressed in various tissues with particularly abundant expression in thymocytes and T cells (Klas C. et al. Int. Immunol., 5:625-630, 1993; Ogasawara J. et al., J. Exp. Med., 181:485-491).

The ligand of CD95, CD95L, is a CD40-kDa type II cell surface glycoprotein and belongs to the TNF family (Suda, T. et al., Cell, 75:1169-1178, 1993). The expression of CD95L appears more tightly controlled, as it has been mainly detected in immune-privileged sites and on activated T cells and NK cells. Binding of CD95L to CD95 generally results in rapid caspase-dependent apoptosis in CD95 bearing cells (Tomohiro Takahashi, et al., Int. Immunol., 6:1567-1574, 1994). As in the case of TNF, human CD95L in the human body is estimated to be in the form of a trimer (Masato Tanaka, et al., EMBO J., 14:1129-1135, 1995).

A role for CD95/CD95L in the immune system was supported by the study of mice that spontaneously developed autoimmune disease, characterized by lymphoproliferation and manifesting in lymphadenopathy and splenomegaly. These mice were found to have defects that resulted in a decreased expression of CD95 (termed lpr mice for lymphoproliferation) (Watanabe-Fukunaga R. et al., Nature, 356:314-317, 1992). Additionally, gld mice (for generalized lymphoproliferative disorder) which carry a mutation in CD95L rendering the protein unable to bind to the receptor were found to have a very similar phenotype (Lynch, D. H., et al., Immunity 1:131-136, 1994; Takahashi T. et al., Cell, 76:969-976, 1994). Recently, mice have been generated that are deficient for CD95L and display an even more sever phenotype than gld mice, again reinforcing the importance of these genes in the elimination of autoreactive lymphoid cells and in the immune system homeostasis (Karray S. et al., J. Immunol., 172:2118-2125, 2004).

Several studies have shown multiples models by which CD95 signalling can regulate T and B cell development, maturation and deletion. During an adaptive immune response to an infection activated T cells are deleted by CD95-mediated apoptosis in a process called activation-induced cell death (AICD). In addition, Fas-mediated apoptosis regulates other cells involved in adaptive immunity such as antigen-presenting cells and is a principal mechanism by which cytotoxic T lymphocytes (CTL) induce apoptosis in cells expressing foreign antigens (Medema, J. P. et al., Eur. J. Immunol., 27:3492-3498, 1997).

In addition to its role in the immune system there is also evidence that CD95/CD95L plays an essential role in the pathogenesis of a variety of diseases which are characterized by either too much or too little apoptosis. It has been suggested that CD95/CD95L plays important role, at least in part, in HIV-induced CD4+ T cell depletion (Katsikis, P. D. J. Exp. Med. 181:2029, 1995; Gehri, R. AIDS, 10:9-16, 1996). Fas-mediated apoptosis has also been implicated in fulminant hepatitis (Song, E. et al. Nat. med. 9:347, 2003), ischemic reperfusion injury (Lee, P. et al. Am. J. Physiol. Heart Circul., 284: H456, 2003), post-ischemic neuronal degeneration (Martin-Villalba A. et al, J. Neurosci. 19:3809-17, 1999), during traumatic brain injury (Qiu J. et al., J. Neurosci., 22:3504-3511, 2002), graft-versus-host-disease (GVHD) (Via, C., et al., J. Immunol., 157:5387-5393, 1996) and in some types of autoimmune diseases (Nishimura-Morita Y. et al., Int. Immunol., 9:1793, 1997). In view of this situation reagents regulating CD95/CD95L pathway, such as agonistic or antagonistic anti-CD95 antibodies, represent candidates for use as a therapeutic agent in these diseases.

Antibody molecules provided herein and obtained by the inventors exhibit notably advantageous properties, as discussed further below, especially F45D9. These antibody molecules were obtained by a combination of techniques in a strategy designed by the inventors and not previously reported.

The present inventors have provided for the first time monoclonal antibody molecules, which may be fully human, which bind with high affinity to the human Fas molecule, as well as to non-human primate (chimpanzee and common marmoset) Fas and inhibit FasL/Fas-mediated apoptosis. Antibody molecules provided herein according to particular aspects of the invention do not induce apoptosis in-vitro and are tolerable at high doses in-vivo in a preclinical safety model, employing common marmosets. Antibody molecules provided herein may antagonise FasL/Fas-mediated apoptosis of for example human and/or common marmoset T cells and B cells in-vitro and in a SCID mouse model in-vivo, using human target cells. Antibody molecules provided may activate signals other than apoptosis-related signalling such as, co-stimulatory signal for activation and proliferation and non-apoptotic Fas-mediated signalling leading to survival. An antibody molecule of the invention, which may be a F(ab′)₂ fragment, may have the property of completely blocking Fas-induced apoptosis upon ≧12% of receptor occupancy.

The excellent properties mean that binding members with the properties of F45D9 are highly advantageous for binding hFas in its physiological setting. As demonstrated herein, F45D9 and other binding members according to the invention may thus be used to bind Fas and inhibit apoptosis. This may be used to treat a disease or disorder such as (1) Graft-Versus-Host Disease (GVHD) (2) HIV-infected individuals, in particular those non treated HIV-infected individuals with decreasing CD4 T cells and low viral load, or anti-viral treated HIV-infected individuals with controlled viral load but not recovered CD4 T counts (3) Stevens-Johnson syndrome (SJS) and Toxic epidermal necrolysis (TEN) (4) Islet transplantation as treatment for insulin-dependent diabetes (autoimmune diabetes) (5) diseases based on ischemia or ischemic reperfusion injury, and in particular, disease based on ischemic reperfusion injury in heart, kidney, liver, lung, gut or brain (ex. stroke); and diseases based on ischemic reperfusion injury associated with surgery or transplantation and ischemic reperfusion injury associated with thrombolytic therapy or angioplasty (6) heart disease, and preferably, ischemic heart diseases, and especially, myocardial infarction; heart failure; and ischemic reperfusion injury (7) renal disease, and preferably, renal failure; renal ischemia; ischemic reperfusion injury and acute renal failure (8) neurological disorders and injuries, particularly cerebral or spinal cord injury, and stroke. (9) lymphocyte depletion in cancer patients associated to cytotoxic antineoplastic therapy.

Binding members according to the present invention are useful in binding to human Fas and preferably, but not limited to, inhibiting Fas-mediated apoptosis, with therapeutic potential in various diseases and disorders in which cells that undergo Fas-mediate apoptosis play a role. Exemplary diseases and disorders are discussed further herein.

### BRIEF DESCRIPTION OF THE FIGURES

FIG. 1A shows the results of binding of F45D9-γ1 and F45D9-γ4 to Jurkat cells with titration at different concentrations as indicated. Diamonds: F45D9-γ1; squares: F45D9-γ4.

FIG. 1B shows the results of experiments determining the reactivity of F45D9-γ1 to the surface of Jurkat cells. The bold solid line indicates staining by F45D9-γ1 mAb and the light solid line represents staining by isotype control antibodies.

FIG. 1C shows the results of binding of F45D9-γ1 and F45D9-γ4 to SKW6.4 cells (titration). Diamonds: F45D9-γ1; squares: F45D9-γ4.

FIG. 2A shows results of experiments demonstrating blocking of antibody binding to the surface of Jurkat cell line by means of pre-incubation with recombinant sFas. Solid blocks: F45D9-γ1; open blocks F45D9-γ1+sFas*.

FIG. 2B shows results of experiments demonstrating blocking of antibody binding to the surface of SKW6.4 cells (malignant human lymphoblastoid B cell) by means of pre-incubation with recombinant sFas. Solid blocks: F45D9-γ1; open blocks F45D9-γ1+sFas*.

FIG. 2C shows histograms of F45D9-γ4 mAb binding to the surface of Jurkat cells expressing different levels of Fas. The bold solid line indicates staining by F45D9-γ4 mAb or anti-CD95 positive control mAb and filled histogram represents staining with control antibodies.

FIG. 3A shows sensorgrams with bivalent analyte fit showing the binding of F45D9-γ1 mAb interaction to Fas at 2.12, 4.25, 17 and 68 3, 6, 33, 66,132 nM mAb concentrations. Sensogram shows the relative response in resonance units after background subtraction vs time in seconds.

FIG. 3B shows sensorgrams with bivalent analyte fit of F45D9-γ4 mAb interaction to Fas at 2.12, 4.25, 17 and 68 nM mAb concentrations. Sensogram shows the relative response in resonance units after background subtraction vs time in seconds. FIG. 3B shows results of application of the BIAcore models to calculate the binding and affinity constants. 1:1 binding model gave a nice fit using 3, 6 and 33 nM.

FIG. 4 shows an alignment of Fas molecule amino acid sequence with amino acid sequence of binding peptides 31, 32 and 33 after epitope mapping analysis using peptide microarrays from JPT peptide technology.

FIG. 5 illustrates the domain structure of Fas and the common region (145-164 aa) of peptides bound by F45D9 antibody molecules. PLAD: pre-ligand-binding assembly domain TM: transmembrane domain.

FIG. 6A shows results of experiments determining apoptosis after Annexin-V and Propidium iodide (PI) staining and flow cytometry analysis, demonstrating that F45D9-γ1 alone does not induce apoptosis in Jurkat cells.

FIG. 6B shows results of experiments determining apoptosis after Annexin-V and Propidium iodide (PI) staining and flow cytometry analysis, demonstrating that F45D9-γ4 alone does not induce apoptosis in Jurkat cells.

FIG. 6C shows results of experiments determining apoptosis after Annexin-V and Propidium iodide (PI) staining and flow cytometry analysis, showing blocking of rFasL-induced apoptosis in Jurkat cells by F45D9-γ1.

FIG. 6D shows results of experiments determining apoptosis after Annexin-V and Propidium iodide (PI) staining and flow cytometry analysis; blocking of rFasL-induced apoptosis in SKW6.4 cells by F45D9-γ1, again showing that F45D9-γ1 alone does not induce apoptosis.

FIG. 6E shows the results of binding of F45D9-γ1, F45D9-γ4, F45D9-γ1 F(ab)₂ or Fab fragments to Jurkat cells with titration at different concentrations as indicated.

FIG. 6F shows results of experiments determining apoptosis after Annexin-V and Propidium iodide (PI) staining and flow cytometry analysis; blocking of rFasL-induced apoptosis in Jurkat cells by F45D9-γ1, F45D9-γ4, F45D9-γ1 F(ab)₂ or Fab fragments.

FIG. 7A shows results of experiments determining apoptosis after Annexin-V and Propidium iodide (PI) staining and flow cytometry analysis, showing blocking of rFasL-induced apoptosis of Activated Human T cells by F45D9-γ1. F45D9-γ1 alone does not induce apoptosis of Activated Human T cells.

FIG. 7B shows results of experiments determining binding of F45D9-γ1 to Fas on Activated Human T cells (titration).

FIG. 7C shows results of experiments determining apoptosis after Annexin-V and Propidium iodide (PI) staining and flow cytometry analysis, showing blocking of rFasL-induced apoptosis of Activated Human T cells by F45D9-γ4. F45D9-γ4 alone does not induce apoptosis of Activated Human T cells.

FIG. 7D shows results of experiments determining binding of F45D9-γ4 to Fas on Activated Human T cells (titration).

FIG. 8 shows images of tissue sections obtained from corresponding treated animals and subject to TUNEL assay to detect cell death. An illustration of the animal treatment procedure preceding use of the assay to detect cell death in tissues is also shown. In TUNEL staining red areas are dead cells.

FIG. 9A shows histograms with results illustrating reactivity of antibodies to Fas antigen on cells from different species quantified by flow cytometry. The bold solid line indicates staining with anti-Fas antibodies and the light solid line represents staining by isotype control antibodies.

FIG. 9B shows histograms with results illustrating reactivity of antibodies to Fas antigen on cells from different species quantified by flow cytometry. The bold solid line indicates staining with anti-Fas antibodies and the light solid line represents staining by isotype control antibodies.

FIG. 9C shows the results of binding of F45D9-γ1 to SKW6.4 cells and two marmoset B cell lines (9505 and 9601) with titration at different concentrations as indicated.

FIG. 9D shows the results of binding of F45D9-γ4 to SKW6.4 cells and two marmoset B cell lines (9505 and 9601) with titration at different concentrations as indicated.

FIG. 9E shows the results of binding of F45D9-γ1 and F45D9-γ4 to PBMC isolated from marmoset animal with titration at different concentrations as indicated.

FIG. 9F shows the results of binding of F45D9-γ1 and F45D9-γ4 to PBMC isolated from human healthy donor with titration at different concentrations as indicated.

FIG. 9G shows immunohistochemistry staining results illustrating reactivity of the F45D9-γ4 (upper sections) and human IgG4 isotype control (lower sections) in human (left panel) and marmoset (right panel) liver tissue.

FIG. 10A shows results of experiments determining blocking of rFasL-induced apoptosis in marmoset B cell line (9505) with F45D9-γ1; F45D9-γ1 alone does not induce apoptosis.

FIG. 10B shows results of experiments determining blocking of rFasL-induced apoptosis in marmoset B cell line (9505) and human B cell line (SKW6.4) with F45D9-γ4 titrated at different concentrations as indicated; F45D9-γ4 alone does not induce apoptosis of marmoset cells.

FIG. 10C shows results of experiments determining apoptosis after Annexin-V and Propidium iodide (PI) staining and flow cytometry analysis (apoptosis of cells in medium alone was substracted), showing blocking of rFasL-induced apoptosis of Activated marmoset lymphocytes (marmoset 1196) by F45D9-γ4 titrated at different concentrations as indicated. F45D9-γ4 alone does not induce apoptosis of activated marmoset lymphocytes.

FIG. 10D shows results of experiments determining apoptosis after Annexin-V and Propidium iodide (PI) staining and flow cytometry analysis (apoptosis of cells in medium alone was substracted), showing blocking of rFasL-induced apoptosis of Activated marmoset lymphocytes (marmoset 1181) by F45D9-γ4 titrated at different concentrations as indicated. F45D9-γ4 alone does not induce apoptosis of activated marmoset lymphocytes.

FIG. 11A shows the percentage of CD25 and CD69 double positive cells on CD4+ T cells in experiments determining F45D9-γ1 effect on activation of human T cells.

FIG. 11B shows the percentage of CD25 and CD69 double positive cells on CD8+ T cells+ in experiments determining F45D9-γ1 effect on activation of human T cells.

FIG. 12A shows results of experiments determining Fas-mediated T cell proliferation, by measuring ³H-thymidine incorporation, and the enhancing effect of F45D9-γ1.

FIG. 12B shows results of experiments determining Fas-mediated T cell proliferation, measured after CFSE staining.

FIG. 13 shows results of experimental determination of antibody dependent cell mediated cytotoxicity (ADCC). Squares: IgG4; circles IgG1.

FIG. 14 shows results of experimental determination of complement dependent cytotoxicity (CDC).

FIG. 15 shows results of determination of in vitro hepatotoxicity of F45D9-γ1 (left panel) and APO-1-3 (right panel—mouse anti-Fas antibody, used as a positive control).

FIG. 16A shows the effect of the human F45D9-γ1 anti-Fas antibody in down-regulating the GvHR in skin tissue sections from three (indicated by arrow) out of six experiments using the skin explants model of human GVHD under mismatch setting and adding F45D9 antibody in MLR and skin explants wells.

FIG. 16B shows the effect of the human F45D9-γ1 anti-Fas antibody in down-regulating the GvHR in skin tissue sections from a representative experimental skin explants model of human GVHD.

FIG. 16C shows results of IL-10 determination in supernatants from MLR treated with F45D9-γ1 or control human IgG1 in a skin explants model of human GVHD.

FIG. 16D shows results of IL-2 determination in supernatants from MLR treated with F45D9-γ1 or control human IgG1 in a skin explants model of human GVHD.

FIG. 16E shows the effect of the human F45D9-γ4 anti-Fas antibody in down-regulating the GvHR in skin tissue sections from four (indicated by arrow) out of nine experiments using the skin explants model of human GVHD under mismatch setting and adding F45D9 antibody in MLR and skin explants wells.

FIG. 17A shows results of experiments determining percentage of specific lysis (results shown as % inhibition of killing) in a ⁵¹Cr release assay showing blocking of Ates B mediated killing of HLA-A2 expressing LCL BK-B5, by F45D9-γ4 mAb titrated at different concentrations as indicated.

FIG. 17B shows results of experiments determining percentage of specific lysis (results shown as % inhibition of killing) in a ⁵¹Cr release assay showing blocking of cytolysis mediated by a CMA treated allogeneic T cell clone (310905/Mon-B1) of BK-B5 targets by F45D9-γ4 mAb titrated at different concentrations as indicated.

FIG. 17C shows results of experiments determining percentage of specific lysis (% of killing) in a ⁵¹Cr release assay showing blocking of Ates B mediated killing of BK-B5 target cells, by F45D9-γ4 mAb, anti-FasL NOK-2 mAb, and Fas-Fc fusion protein, titrated at different concentrations as indicated.

The following sequences are disclosed herein:

SEQ ID NO. 1 F45D9 VH encoding nucleotide sequence

SEQ ID NO. 2 F45D9 VH amino acid sequence

SEQ ID NO. 3 F45D9 VL encoding nucleotide sequence

SEQ ID NO. 4 F45D9 VL amino acid sequence

SEQ ID NO. 5 F45D9 VH CDR1 amino acid sequence

SEQ ID NO. 6 F45D9 VH CDR2 amino acid sequence

SEQ ID NO. 7 F45D9 VH CDR3 amino acid sequence

SEQ ID NO. 8 F45D9 VL CDR1 amino acid sequence

SEQ ID NO. 9 F45D9 VL CDR2 amino acid sequence

SEQ ID NO. 10 F45D9 VL CDR3 amino acid sequence

SEQ ID NO. 11 Human Fas antigen amino acid sequence

SEQ ID NO. 12 JPT31 peptide amino acid sequence

SEQ ID NO. 13 JPT32 peptide amino acid sequence

SEQ ID NO. 14 JPT33 peptide amino acid sequence

SEQ ID NO. 15 JPT31, 32 and 33 peptide common region amino acid sequence

SEQ ID NO. 16 F45D9-IgG4 heavy chain amino acid sequence

In one aspect, the present invention provides a binding member which binds human Fas and which comprises the F45D9 VH domain (SEQ ID NO. 2) and/or the F45D9 VL domain (SEQ ID NO. 4)

Generally, a VH domain is paired with a VL domain to provide an antibody antigen binding site, although as discussed further below a VH domain alone may be used to bind antigen. In one preferred embodiment, the F45D9 VH domain (SEQ ID NO. 2) is paired with the F45D9 VL domain (SEQ ID NO. 4), so that an antibody antigen binding site is formed comprising both the F45D9 VH and VL domains. In other embodiments, the F45D9 VH is paired with a VL domain other than the F45D9 VL. Light-chain promiscuity is well established in the art.

One or more CDRs may be taken from the F45D9 VH or VL domain and incorporated into a suitable framework. This is discussed further below. F45D9 VH CDR's 1, 2 and 3 are shown in SEQ ID NO.'s 5, 6 and 7, respectively. F45D9 VL CDR's 1, 2 and 3 are shown in SEQ ID NO.'s 8, 9 and 10, respectively.

Variants of the VH and VL domains and CDRs of which the sequences are set out herein and which can be employed in binding members for human Fas can be obtained by means of methods of sequence alteration or mutation and screening. Such methods are also provided by the present invention.

Variable domain amino acid sequence variants of any of the VH and VL domains whose sequences are specifically disclosed herein may be employed in accordance with the present invention, as discussed. Particular variants may include one or more amino acid sequence alterations (addition, deletion, substitution and/or insertion of an amino acid residue), maybe less than about 20 alterations, less than about 15 alterations, less than about 10 alterations or less than about 5 alterations, 4, 3, 2 or 1. Alterations may be made in one or more framework regions and/or one or more CDR's.

A binding member according to the invention may be one which competes for binding to antigen with any binding member which both binds the antigen and comprises a binding member, VH and/or VL domain disclosed herein, or VH CDR3 disclosed herein, or variant of any of these. Competition between binding members may be assayed easily in vitro, for example using ELISA and/or by tagging a specific reporter molecule to one binding member which can be detected in the presence of other untagged binding member(s), to enable identification of binding members which bind the same epitope or an overlapping epitope.

Thus, a further aspect of the present invention provides a binding member comprising a human antibody antigen-binding site which competes with F45D9 for binding to human Fas.

Various methods are available in the art for obtaining antibodies against human Fas and which may compete with F45D9 for binding to human Fas.

As noted, the epitope recognised by F45D9 is within SNTKCKEEGSRSNLGWLCLL (SEQ ID NO.15). The invention provides binding members that bind the peptide of SEQ ID NO: 12, 13 and/or 14 or a fragment of any one or more thereof that is bound by F45D9, and binding members that compete with F45D9 for binding to the peptide of SEQ ID NO: 12, 13 and/or 14 or a fragment of any one or more thereof bound by F45D9.

Binding members of the present invention may do one or more or any combination of any of the following:
bind human Fas;
bind non-human primate common marmoset Fas;
bind non-human primate chimpanzee Fas;
bind a peptide with sequence of SEQ ID NO. 12 or a fragment thereof that is bound by F45D9;
bind a peptide with sequence of SEQ ID NO. 13 or a fragment thereof that is bound by F45D9;
bind a peptide with sequence of SEQ ID NO. 14 or a fragment thereof that is bound by F45D9;
compete with F45D9 for binding to the peptide of SEQ ID NO. 12 or a fragment thereof bound by F45D9;
compete with F45D9 for binding to the peptide of SEQ ID NO. 13 or a fragment thereof bound by F45D9;
compete with F45D9 for binding to the peptide of SEQ ID NO. 14 or a fragment thereof bound by F45D9;
inhibit or antagonise human Fas-mediated apoptosis of cells, e.g. T cells and/or B cells, in vitro and/or in a SCID mouse model, the cells being human or non-human e.g. common marmoset;
inhibit or antagonise rhFasL-induced apoptosis of cells, e.g. T and/or B cells, in vitro and/or in a SCID mouse model, the cells being human or non-human e.g. common marmoset;
mediate a co-stimulatory signal, e.g. with an anti-CD3 antibody molecule, in the proliferation of human T cells;
do not induce complement dependent cytotoxicity;
do not induce complement dependent cytotoxicity at a concentration of 0.15 ug/ml to 20 ug/ml;
do not induce hepatotoxicity in primary human hepatocytes, e.g. as determined using XTT assay;
do not induce hepatotoxicity in primary human hepatocytes, e.g. as determined using XTT assay, at a concentration in the range of 0.1 ug/ml to 10 ug/ml.
induce antibody dependent cell mediated cytotoxicity (ADCC) in the form of IgG1 isotype;
do not induce antibody dependent cell mediated cytotoxicity (ADCC) in the form of IgG4 isotype;
inhibit the GvHR in skin tissue sections from experimental skin explants model of human GVHD;
Inhibit or antagonise human cytotoxic T cell (CTL) activity.

In a further aspect, the present invention provides a method of obtaining one or more binding members able to bind the antigen, the method including bringing into contact a library of binding members according to the invention and said antigen, and selecting one or more binding members of the library able to bind said antigen.

The library may be displayed on the surface of bacteriophage or other biological particles, each particle containing nucleic acid encoding the antibody VH variable domain displayed on its surface, and optionally also a displayed VL domain if present. Alternatives include ribosome or peptide display, whereby the antibody variable domains are bound to a selectable material to which encoding nucleic acid is also bound.

Following selection of binding members able to bind the antigen and displayed on bacteriophage or other particles, or bound to ribosomes or other selectable material, nucleic acid may be taken from the particle, ribosome or other selectable material displaying or bound to a said selected binding member. Such nucleic acid may be used in subsequent production of a binding member or an antibody VH variable domain (optionally an antibody VL variable domain) by expression from nucleic acid with the sequence of nucleic acid taken from the particle displaying a said selected binding member or other selectable material to which the selected binding member was bound.

An antibody VH variable domain with the amino acid sequence of an antibody VH variable domain of a said selected binding member may be provided in isolated form, as may a binding member comprising such a VH domain.

Ability to bind human Fas may be further tested, also ability to compete with a binding member with an antigen binding site composed of the F45D9 VH and F45D9 VL domains for binding to human Fas. Ability to antagonise action of Fas may be tested, as discussed further below.

A binding member according to the present invention may bind human Fas with the affinity of F45D9. Affinity of a binding member in accordance with the present invention may be determined by BIAcore, Fas specific ELISA and/or Flow cytometry analysis of antibody binding to Fas molecule on the surface of Jurkat cell line.

A binding member according to the present invention may inhibit apoptosis with the potency of F45D9. This may be measure by Annexin-V and Propidium iodide staining and flow cytometry analysis of recombinant FasL-induced apoptosis of Jurkat cell line.

Binding affinity and potency of different binding members can be compared under appropriate conditions.

A binding member according to the present invention may inhibit Fas-mediated apoptosis which can be measured by Annexin-V and Propidium iodide staining and flow cytometry analysis of recombinant FasL-induced apoptosis of Jurkat cell line.

In addition to antibody sequences, a binding member according to the present invention may comprise other amino acids, e.g. forming a peptide or polypeptide, such as a folded domain, or to impart to the molecule another functional characteristic in addition to ability to bind antigen. Binding members of the invention may carry a detectable label, or may be conjugated to a toxin or enzyme (e.g. via a peptidyl bond or linker).

In further aspects, the invention provides an isolated nucleic acid which comprises a sequence encoding a binding member, VH domain and/or VL domain according to the present invention, and methods of preparing a binding member, a VH domain and/or a VL domain of the invention, which comprise expressing said nucleic acid under conditions to bring about production of said binding member, VH domain and/or VL domain, and recovering it.

Binding members according to the invention may be used in a method of treatment or diagnosis of the human or animal body, such as a method of treatment (which may include prophylactic treatment) of a disease or disorder in a human patient which comprises administering to said patient an effective amount of a binding member of the invention. Conditions treatable in accordance with the present invention include those discussed elsewhere herein. The present invention provides a composition comprising a binding member of the invention for use in such methods of diagnosis or treatment and for the use of a binding member of the invention in the manufacture of a medicament for diagnosis or treatment in accordance with such methods.

A further aspect of the present invention provides nucleic acid, generally isolated, encoding an antibody VH variable domain and/or VL variable domain disclosed herein.

Another aspect of the present invention provides nucleic acid, generally isolated, encoding a VH CDR or VL CDR sequence disclosed herein, especially a VH CDR selected from SEQ ID NO.'s 5, 6 and 7 or a VL CDR selected from SEQ ID NO.'s 8, 9 and 10, most preferably F45D9 VH CDR3 (SEQ ID NO. 7).

A further aspect provides a host cell transformed with nucleic acid of the invention.

A yet further aspect provides a method of production of an antibody VH variable domain, the method including causing expression from encoding nucleic acid. Such a method may comprise culturing host cells under conditions for production of said antibody VH variable domain.

Analogous methods for production of VL variable domains and binding members comprising a VH and/or VL domain are provided as further aspects of the present invention.

A method of production may comprise a step of isolation and/or purification of the product.

A method of production may comprise formulating the product into a composition including at least one additional component, such as a pharmaceutically acceptable excipient.

These and other aspects of the invention are described in further detail below.

### Terminology

### Binding Member

This describes a member of a pair of molecules that bind one another. The members of a binding pair may be naturally derived or wholly or partially synthetically produced. One member of the pair of molecules has an area on its surface, or a cavity, which binds to and is therefore complementary to a particular spatial and polar organisation of the other member of the pair of molecules. Thus the members of the pair have the property of binding to each other. Examples of types of binding pairs are antigen-antibody, biotin-avidin, hormone-hormone receptor, receptor-ligand, enzyme-substrate. This application is concerned with antigen-antibody type reactions.

### Antibody Molecule

This describes an immunoglobulin whether natural or partly or wholly synthetically produced. The term also covers any polypeptide or protein comprising an antibody binding domain. Antibody fragments which comprise an antigen binding domain are such as Fab, scFv, Fv, dAb, Fd; and diabodies.

It is possible to take monoclonal and other antibodies and use techniques of recombinant DNA technology to produce other antibodies or chimeric molecules which retain the binding ability of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB 2188638A or EP-A-239400. A hybridoma or other cell producing an antibody may be subject to genetic mutation or other changes, which may or may not alter the binding specificity of antibodies produced.

As antibodies can be modified in a number of ways, the term “antibody molecule” should be construed as covering any binding member or substance having an antibody antigen-binding domain with the required binding for epitope or antigen. Thus, this term covers antibody fragments and derivatives, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) bispecific single chain Fv dimers (PCT/US92/09965) and (ix) “diabodies”, multivalent or multispecific fragments constructed by gene fusion (WO94/13804; P. Holliger et al, Proc. Natl. Acad. Sci. USA 90 6444-6448, 1993). Fv, scFv or diabody molecules may be stabilised by the incorporation of disulphide bridges linking the VH and VL domains (Y. Reiter et al, Nature Biotech, 14, 1239-1245, 1996). Minibodies comprising a scFv joined to a CH3 domain may also be made (S. Hu et al, Cancer Res., 56, 3055-3061, 1996).

Where bispecific antibodies are to be used, these may be conventional bispecific antibodies, which can be manufactured in a variety of ways (Holliger, P. and Winter G. Current Opinion Biotechnol. 4, 446-449 (1993)), e.g. prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against Fas, then a library can be made where the other arm is varied and an antibody of appropriate specificity selected. Bispecific whole antibodies may be made by knobs-into-holes engineering (J. B. B. Ridgeway et al, Protein Eng., 9, 616-621, 1996).

### Antigen Binding Domain

This describes the part of an antibody molecule which comprises the area which binds to and is complementary to part or all of an antigen. Where an antigen is large, an antibody may only bind to a particular part of the antigen, which part is termed an epitope. An antigen binding domain may be provided by one or more antibody variable domains (e.g. a so-called Fd antibody fragment consisting of a VH domain). Preferably, an antigen binding domain comprises an antibody light chain variable region (VL) and an antibody heavy chain variable region (VH).

### Comprise

This is generally used in the sense of include, that is to say permitting the presence of one or more features or components.

### Isolated

This refers to the state in which binding members of the invention, or nucleic acid encoding such binding members, will generally be in accordance with the present invention. Members and nucleic acid will be free or substantially free of material with which they are naturally associated such as other polypeptides or nucleic acids with which they are found in their natural environment, or the environment in which they are prepared (e.g. cell culture) when such preparation is by recombinant DNA technology practised in vitro or in vivo. Members and nucleic acid may be formulated with diluents or adjuvants and still for practical purposes be isolated—for example the members will normally be mixed with gelatin or other carriers if used to coat microtitre plates for use in immunoassays, or will be mixed with pharmaceutically acceptable carriers or diluents when used in diagnosis or therapy. Binding members may be glycosylated, either naturally or by systems of heterologous eukaryotic cells (e.g. CHO or NS0 (ECACC 85110503) cells, or they may be (for example if produced by expression in a prokaryotic cell) unglycosylated.

By “substantially as set out” it is meant that the relevant CDR or VH or VL domain of the invention will be either identical or highly similar to the specified regions of which the sequence is set out herein. By “highly similar” it is contemplated that from 1 to 5, preferably from 1 to 4 such as 1 to 3 or 1 or 2, or 3 or 4, amino acid substitutions may be made in the CDR and/or VH or VL domain.

The structure for carrying a CDR of the invention will generally be of an antibody heavy or light chain sequence or substantial portion thereof in which the CDR is located at a location corresponding to the CDR of naturally occurring VH and VL antibody variable domains encoded by rearranged immunoglobulin genes. The structures and locations of immunoglobulin variable domains may be determined by reference to (Kabat, E. A. et al, Sequences of Proteins of Immunological Interest. 4th Edition. US Department of Health and Human Services. 1987, and updates thereof, now available on the Internet (http://immuno.bme.nwu.edu or find “Kabat” using any search engine).

Preferably, a CDR amino acid sequence substantially as set out herein is carried as a CDR in a human variable domain or a substantial portion thereof. The VH CDR3 sequences substantially as set out herein represent preferred embodiments of the present invention and it is preferred that each of these is carried as a VH CDR3 in a human heavy chain variable domain or a substantial portion thereof.

Variable domains employed in the invention may be obtained from any germ-line or rearranged human variable domain, or may be a synthetic variable domain based on consensus sequences of known human variable domains. A CDR sequence of the invention (e.g. CDR3) may be introduced into a repertoire of variable domains lacking a CDR (e.g. CDR3), using recombinant DNA technology.

For example, Marks et al (Bio/Technology, 1992, 10:779-783) describe methods of producing repertoires of antibody variable domains in which consensus primers directed at or adjacent to the 5′ end of the variable domain area are used in conjunction with consensus primers to the third framework region of human VH genes to provide a repertoire of VH variable domains lacking a CDR3. Marks et al further describe how this repertoire may be combined with a CDR3 of a particular antibody. Using analogous techniques, the CDR3-derived sequences of the present invention may be shuffled with repertoires of VH or VL domains lacking a CDR3, and the shuffled complete VH or VL domains combined with a cognate VL or VH domain to provide binding members of the invention. The repertoire may then be displayed in a suitable host system such as the phage display system of WO92/01047 so that suitable binding members may be selected. A repertoire may consist of from anything from 10⁴ individual members upwards, for example from 10⁶ to 10⁸ or 10¹⁰ members.

Analogous shuffling or combinatorial techniques are also disclosed by Stemmer (Nature, 1994, 370:389-391), who describes the technique in relation to a β-lactamase gene but observes that the approach may be used for the generation of antibodies.

A further alternative is to generate novel VH or VL regions carrying a CDR-derived sequences of the invention using random mutagenesis of one or more selected VH and/or VL genes to generate mutations within the entire variable domain. Such a technique is described by Gram et al (1992, Proc. Natl. Acad. Sci., USA, 89:3576-3580), who used error-prone PCR.

Another method which may be used is to direct mutagenesis to CDR regions of VH or VL genes. Such techniques are disclosed by Barbas et al, (1994, Proc. Natl. Acad. Sci., USA, 91:3809-3813) and Schier et al (1996, J. Mol. Biol. 263:551-567).

Human antibody technology, specifically HuMAb-Mouse technology by Medarex was used in the present invention to generate F45D9 antibody. In these transgenic mice, the mouse genes for creating antibodies have been inactivated and replaced by human antibody genes. HuMAb-Mouse transgenic strains contain key gene sequences from unrearranged human antibody genes that code for both the heavy and light chains of human antibodies. Then, these transgenic mice make human antibody proteins. This avoids the need to humanize murine monoclonal antibodies, and because the human genes in HuMAb-Mouse are stable, they are passed on to offspring of the mice. Mice can, therefore, be bred indefinitely at relative low cost and without additional genetic engineering.

All the above described techniques are known as such in the art and in themselves do not form part of the present invention. The skilled person will be able to use such techniques to provide binding members of the invention using routine methodology in the art.

A further aspect of the invention provides a method for obtaining an antibody antigen-binding domain for human Fas antigen, the method comprising providing by way of addition, deletion, substitution or insertion of one or more amino acids in the amino acid sequence of a VH domain set out herein a VH domain which is an amino acid sequence variant of the VH domain, optionally combining the VH domain thus provided with one or more VL domains, and testing the VH domain or VH/VL combination or combinations for to identify a binding member or an antibody antigen binding domain for human Fas and optionally with one or more of preferred properties, preferably ability to inhibit Fas-mediated apoptosis. Said VL domain may have an amino acid sequence which is substantially as set out herein.

An analogous method may be employed in which one or more sequence variants of a VL domain disclosed herein are combined with one or more VH domains.

A further aspect of the invention provides a method of preparing a binding member for human Fas, which method comprises:
(a) providing a starting repertoire of nucleic acids encoding a VH domain which either include a CDR3 to be replaced or lack a CDR3 encoding region;
(b) combining said repertoire with a donor nucleic acid encoding an amino acid sequence substantially as set out herein for a VH CDR3 such that said donor nucleic acid is inserted into the CDR3 region in the repertoire, so as to provide a product repertoire of nucleic acids encoding a VH domain;
(c) expressing the nucleic acids of said product repertoire;
(d) selecting a binding member for a Fas; and
(e) recovering said binding member or nucleic acid encoding it.

Again, an analogous method may be employed in which a VL CDR3 of the invention is combined with a repertoire of nucleic acids encoding a VL domain which either include a CDR3 to be replaced or lack a CDR3 encoding region.

Similarly, one or more, or all three CDRs may be grafted into a repertoire of VH or VL domains which are then screened for a binding member or binding members for Fas.

A substantial portion of an immunoglobulin variable domain will comprise at least the three CDR regions, together with their intervening framework regions. Preferably, the portion will also include at least about 50% of either or both of the first and fourth framework regions, the 50% being the C-terminal 50% of the first framework region and the N-terminal 50% of the fourth framework region. Additional residues at the N-terminal or C-terminal end of the substantial part of the variable domain may be those not normally associated with naturally occurring variable domain regions. For example, construction of binding members of the present invention made by recombinant DNA techniques may result in the introduction of N- or C-terminal residues encoded by linkers introduced to facilitate cloning or other manipulation steps. Other manipulation steps include the introduction of linkers to join variable domains of the invention to further protein sequences including immunoglobulin heavy chains, other variable domains (for example in the production of diabodies) or protein labels as discussed in more details below.

Although in a preferred aspect of the invention binding members comprising a pair of VH and VL domains are preferred, single binding domains based on either VH or VL domain sequences form further aspects of the invention. It is known that single immunoglobulin domains, especially VH domains, are capable of binding target antigens in a specific manner.

In the case of either of the single chain binding domains, these domains may be used to screen for complementary domains capable of forming a two-domain binding member able to bind Fas.

This may be achieved by phage display screening methods using the so-called hierarchical dual combinatorial approach as disclosed in WO92/01047 in which an individual colony containing either an H or L chain clone is used to infect a complete library of clones encoding the other chain (L or H) and the resulting two-chain binding member is selected in accordance with phage display techniques such as those described in that reference. This technique is also disclosed in Marks et al, ibid.

Binding members of the present invention may further comprise antibody constant regions or parts thereof. For example, a VL domain may be attached at its C-terminal end to antibody light chain constant domains including human Cκ or Cλ chains, preferably Cκ chains. Similarly, a binding member based on a VH domain may be attached at its C-terminal end to all or part of an immunoglobulin heavy chain derived from any antibody isotype, e.g. IgG, IgA, IgE and IgM and any of the isotype sub-classes, particularly IgG1 and IgG4, preferred is IgG4. Fc regions such as Δnab and Δnac as disclosed in WO99/58572 may be employed.

It is well established in the literature that the native IgG4 isotype forms functionally monovalent antibody species caused by dynamic Fab arm exchange (Van der Neut Kolfschoten 2007, Science, 317,1554). This property of the IgG4 antibody has been ascribed an intrinsic instability of the inter-heavy chain disulphide bonds of the molecule leading to an equilibrium with IgG4 half molecules. To circumvent this unwanted property (from a drug development and manufacturing point of view) a mutation was previously introduced by researchers (Angal 1993, Mol. Immunol., 30, 105) and was shown to stabilize the IgG4 antibody (Schuurman 2001, Mol. Immunol., 38, 1; Aalberse 2002, Immunol., 105, 9). This mutation corresponds to the more stable IgG1 like hinge sequence Cys-Pro-Pro-Cys as compared to the native IgG4 Cys-Pro-Ser-Cys sequence (S228P mutation; EU index), apparently stabilizing the adjacent inter-chain disulphide bond.

A preferred Fc region employed in different aspects and embodiments of the present invention is of IgG4 isotype containing the S228P mutation (EU index), such that the hinge sequence Cys-Pro-Pro-Cys is present instead of the native IgG4 Cys-Pro-Ser-Cys sequence.

Binding members of the invention may be labelled with a detectable or functional label. Examples of detectable labels include radiolabels such as ¹³¹I or ⁹⁹Tc, which may be attached to antibodies of the invention using conventional chemistry known in the art of antibody imaging, enzyme labels such as horseradish peroxidise, chemical moieties such as biotin which may be detected via binding to a specific cognate detectable moiety, e.g. labelled avidin, and fluorochromes, e.g. fluorescein isothiocyanate (FITC).

Binding members of the present invention are designed to be used in methods of diagnosis or treatment in human or animal subjects, preferably human.

Accordingly, further aspects of the invention provide methods of treatment comprising administration of a binding member as provided, compositions comprising a binding member for use in such methods, pharmaceutical compositions comprising such a binding member, and use of such a binding member in the manufacture of a medicament for administration, for example in a method of making a medicament or pharmaceutical composition comprising formulating the binding member with a pharmaceutically acceptable excipient.

Clinical indications in which an anti-Fas antibody may be used to provide therapeutic benefit include any condition in which apoptosis and/or Fas has pathological consequences, for example in (1) GVHD (2) HIV-infected individuals, in particular, those non treated HIV-infected individuals with decreasing CD4 T cells and low viral load, as wells as anti-viral treated HIV-infected individuals that controlled viral load but not recovered CD4 T counts (3) Stevens-Johnson syndrome (SJS) and Toxic epidermal necrolysis (TEN) (4) Islet transplantation as treatment for insulin-dependent diabetes (autoimmune diabetes) (5) diseases based on ischemia or ischemic reperfusion injury, and in particular, disease based on ischemic reperfusion injury in heart, kidney, liver, lung, gut or brain (ex. stroke); and diseases based on ischemic reperfusion injury associated with surgery or transplantation and ischemic reperfusion injury associated with thrombolytic therapy or angioplasty (6) heart disease, and preferably, ischemic heart diseases, and especially, myocardial infarction; heart failure; and ischemic reperfusion injury (7) renal disease, and preferably, renal failure; renal ischemia; ischemic reperfusion injury and acute renal failure (8) neurological disorders and injuries, particularly cerebral or spinal cord injury, and stroke. (9) lymphocyte depletion in cancer patients associated to cytotoxic antineoplastic therapy.

In addition diseases and other clinical conditions involving cell death may be ameliorated.

Graft-versus-host disease (hereinafter referred to as GVHD) is a disease caused by graft versus host reaction (GVH reaction), which is an immunoreaction that may occur upon transplantation of lymphocytes of a donor or a graft, against the tissue antigens of the host. Exemplary GVHDs are GVHD after bone marrow transplantation, such as with allogenic bone marrow transplantation or with bone marrow transplantation in congenital immune deficiency syndrome; GVHD after organ transplantation; GVHD after blood transfusion, in which a large amount of blood is transfused to a patient of hypoimmunity; and the like. GVHD is associated with organ or tissue failure based on GVH reaction, and diarrhea, exhaustion such as weight loss and thinning, exanthema, splenomegaly, and liver dysfunction are clinically observed. GVHD is also associated with histological symptoms such as disorganization of bone marrow and lymphoid tissue and atrophy of intestinal villi.

Graft-versus-host disease (GVHD) is a complication associated with hematopoietic cell transplantation from allogenic donors. The complex physiopathology of acute GVHD (aGVHD) has been described as a 3-phase process (reviews: Jaksch and Mattsson, Scand. J. Immunol., 61:398, 2005; Shlomchick, W. D., Nat. Rev. Immunol. 7:340, 2007). Phase I is a direct consequence of conditioning before the stem-cell transplant (SCT) that consists in chemotherapy and/or irradiation. These treatments are toxic to the patient's tissues, leading to injuries and cell activation. During this phase pro-inflammatory cytokines are secreted, such as tumor necrosis factor (TNF)-α and interleukin (IL)-1. Phase II occurs after the transplantation of donor hematopoietic stem cells and T cells into the recipient when donor cells become activated. The disparity between donor and recipient major histocompatibility complex (MHC) remains the primary cause for the donor T cell activation. However, even in human leukocyte antigen (HLA)-identical pairs, the differences in minor antigens can lead to a T cell response. Phases I and II lead to Phase III, the effector phase. The main cells responsible for the effector phase are the cytotoxic T lymphocytes (CTL) which infiltrate and damage the tissues. Fas is a crucial molecule, together with perforin and granzyme, for cytotoxic T-lymphocytes (CTL) to kill their targets. Besides cell mediated cytotoxicity, an inflammatory reaction following the release of TNF-α, IFN-γ, IL-1 and nitric oxide (NO) is also responsible for tissue injuries.

Fas is broadly expressed, including on the characteristic GVHD target organs: skin, liver, intestine and thymus. Experimental animal models demonstrate a critical role of Fas/FasL pathway in the physiopathology of GVHD. When Fas or FasL are deficient on host cells, an increased morbidity and mortality was observed in transplanted mice (van den Brink, M. R., et al., Transplantation, 70:184, 2000; van den Brink, M. R., et al., J. Immunol. 164: 469, 2000). However, either by infusing FasL deficient donor cells or by blocking the pathway with FasL neutralizing antibodies, GVHD was markedly reduced (Baker, M. B, et al., J. Exp. Med., 183:2645, 1996; Via, C. S., et al. J. Immunol. 157: 5387, 1996). By treating mice suffering from aGVHD with a combination of anti-FasL and anti-TNF-α antibodies, Hattori et al. observed a complete inhibition of mortality and a decrease of lesions (Hattori, K. et al., Blood, 91:4051, 1998). When administrated separately the anti-FasL antibody was more potent on hepatic lesions, anti-TNF-α improved the intestinal lesions, while both antibodies acted on cutaneous and splenic lesions. Using different mice strains and anti-FasL monoclonal antibody, Miwa et al. confirmed reduced mortality and weight loss in treated mice compared to controls, although they did not report any significant improvement for the other signs of aGVHD, including skin lesions (Miwa, K. et al., Int. Immunol., 11:925, 1999). In human studies, Fas upregulation has been observed and associated with GVHD in gastrointestinal tract (Socie, G., et al., Blood, 103:50, 2004). Based on these studies the potential use of Fas as a therapeutic target in GVHD has been postulated (French and Tschopp, Schweiz Med. Wochenschr., 130:1656, 2000). Furthermore, as compare to blocking perforin/granzyme or TNF-α, targeting Fas would have the beneficial effect of leaving the graft-versus-leukaemia (GVL) effect unaffected as it has been shown in animal studies (Schmaltz, C. et al., Blood, 97:2886, 2001). GVHD grades I-II in the clinical setting are regarded as potentially curative i.e. by immunosuppression and the disease can be under control. However if this worsens to grade III-IV GVHD the disease is very difficult to treat and is life-threatening. In fact there are very few drugs which may be able to reverse this on-going immune reaction and then the responses to the drugs are often only transitory. In steroid refractory GVHD again there are few means of treatment—one is extracorporeal phototherapy—but few drugs are consistently useful as therapeutics in this situation.

Activated human T cells are induced to undergo apoptosis upon triggering through CD3/T cell receptor complex, a process called Activation Induced Cell Death (AICD). AICD is observed in T cells freshly isolated from HIV-infected, but not form uninfected individuals (Groux, et al. J. Exp. Med. 175: 331, 1992). Thus, apoptosis seems to play a role in the depletion of CD4 T cells and progression to AIDS in HIV infected individuals. The therapeutic intervention for HIV-infected individuals with a Fas antagonist, thus may be possible, in particular, those non treated HIV infected individuals with decreasing CD4 T cells and low viral load, as wells as anti-viral treated HIV infected individuals that controlled viral load but not recovered CD4 T counts.

Stevens-Johnson syndrome (SJS) and Toxic epidermal necrolysis (TEN) are severe adverse drug reactions characterized by a low incidence but high mortality. Studies of the pathogenesis of TEN suggest that destruction in epidermis is due to Fas-mediated keratinocyte apoptosis, and probably apoptosis of keratinocytes is also involved in SJS. IVIG inhibits Fas-FasL interaction and cell death in vitro, and thus provides a rationale for use in humans (French, L. E Allergology Int. 55:9, 2006).

Islet transplantation is an effective treatment for insulin-dependent diabetes (autoimmune diabetes). Two major obstacles to successful islet transplantation are Islet Primary nonfunction (PNF) and graft rejection. PNF is defined as loss of islet function after transplantation for reasons other than graft rejection. Fas-mediated apoptosis plays an important role in Islet primary nonfunction. FasL induces apoptosis in Beta cells in vitro and in Fas or FasL deficient mice islet transplantation was more efficient (Wu, Y., Diabetes, 52:2279, 2003).

Ischemic reperfusion injury is found in practically all tissues and organs, and is involved in various diseases. Ischemic reperfusion injury is also a problem in preservation and transplantation of organs. Among such ischemic reperfusion injuries, those associated with infarction of liver, heart, kidney or brain and those associated with surgery or transplantation, and in particular, tissue injury and dysfunction (such as cardiac arrhythmia) in the particular organ may lead to the death of the individual when they are serious, and therefore, such cases are a serious social problem. It is known that organ preservation and reperfusion in the course of organ transplantation is associated with the occurrence of the apoptosis. In addition, modulation of Fas or FasL expression has been reported in some experimental models and increasing evidences implicates Fas-mediated apoptosis in extending infarct size during reperfusion of ischemic tissue in multiple tissues, including the brain, heart, kidney and gut (Martin-Villalba, et al. Cell Death Differ., 8:679, 2001; Hamar P. et al. PNAS, 101: 14883, 2004; Castaneda, M. P: et al., Transplantation, 76:50, 2003. Lung ischemia-reperfusion injury is the inciting event in acute lung failure following transplantation, surgery and shock. Fas deficient mice did show apoptosis induced during in vivo isquemia-reperfusion lung injury. In addition anti-FasL antibody inhibited apoptosis induced during in vitro lung anoxia (Zang, X. et al. J. Biol. Chem, 278:22061, 2003).

Apoptosis of cardiomyocytes, and in particular the involvement of Fas-mediated apoptosis, has been shown to be associated to heart diseases in several experimental models. Apoptosis of cardiomyocytes was found in canine heart failure and myocardial infarction model, in association with an increase in Fas expression (Kajstura J, Lab. Invest. 74:86, 1996; Lab. Invest. 73: 771, 1995). It has also been shown the suppressive effect of one anti-FasL antibody for myocardial infarction lesion in experimental rat model of heart ischemic reperfusion injury model (U.S. Pat. No. 7,128,905 B2). Hearts isolated from lpr mice (lacking functional Fas) or hearts from an in vivo ischemia reperfusion model with the same mice displayed market reduction in cell death after ischemia and reperfusion compared with wild type controls (Jeremias I, et al., Circulation, 102: 915, 2000; Lee P., et al. Am. J. Physiol Heart Circ. Physiol. 284: H456, 2003).

Involvement of apoptosis is also indicated for renal diseases, and increase in Fas mRNA expression is reported in an experimental model of renal ischemic reperfusion injury and small interfering RNA targeting Fas protects mice against renal ischemia-reperfusion injury (Hamar P. et al. PNAS, 101: 14883, 2004). Mice lacking Fas expression, have less kidney tissue damage after ischemia-reperfusion than wild-type mice (Miyazawa, S. et al., J. Lab. Clin. Med., 139:269, 2002).

Hepatic ischemia-reperfusion injury remains a significant problem for liver surgery, including transplantation and apoptosis has been implicated in this type of hepatic injury. Blockage of Fas/FasL interaction with anti-Fas or neutralizing anti-FasL antibodies suppresses hepatocyte apoptosis, hepatic infiltration of macrophages and NK cells as well as liver injury in ischemic-reperfusion rat liver model (Nakajima H., Apoptosis, 13: 1013, 2008). Although hepatic ischemia-reperfusion injury often occurs in liver surgery for trauma, cancer, or transplantation and is clinically a serious problem, an effective regime for treatment remains elusive due to its complex mechanism of onset.

Apoptotic cell death contributes to secondary damage and neurological dysfunction following spinal cord injury (SCI). Main inducers of the apoptotic program in other neurodegenerative models, such as stroke (Martin-Villalba, et al. Cell Death Differ., 8:679, 2001), are the TNF and FasL/Fas system. Following SCI, expression of TNF, Fas and FasL is increased at the lesion site. Expression of Fas was found in astrocytes, oligodendrocytes and microglia following cervical SCI (Casha, W. R., et al. Neuroscience, 103: 203, 2001). In a rodent model of SCI the therapeutic neutralization of FasL alone or of both Fas and TNF significantly improved the clinical outcome and promotes functional recovery after spinal cord injury (US 2006/0234968 A1). This treatment decreased apoptotic cell death following SCI. Neutralization of FasL in this study protects neurons and inhibits demyelination. The spinal and cerebral trauma account for the majority of cases of death and permanent disabilities in the population under the age of 40. The consequences for the society are devastating. Currently, the strategies aimed at repairing spinal cord lesions focus either on neuroprotection, enhanced regeneration, or treatment of demyelination. Given the complexity of spinal cord injury, multiple interventions targeting the different sources of damage, would be required. Neutralization of FasL/Fas system, alone of together with other type of treatment, may offer a new therapy for human spinal trauma.

Although cancer itself is immunosuppressive, cytotoxic antineoplastic therapy is the primary contributor to the clinical immunodeficiency observed in cancer patients. The immunodeficiency induced by cytotoxic antineoplastic therapy is primarily related to T-cell depletion, especially in CD4 T cells. Dose-intensive chemotherapy in cancer patients induced dramatic T-cell depletion associated with activation of lymphocytes and higher susceptibility to apoptosis, suggesting AICD as possible mechanisms in the CD4 depletion (Mackall C. L. et al. Blood, 96:754, 2000; Mackall C. L., Stem Cells 18:10, 2000). Specific approaches are needed to enhance immune reconstitution during chemotherapy to face opportunistic infections and eradication of residual tumors. Therefore, treatment with blocking anti-Fas antibody would ameliorate severe, prolonged CD4+ depletion associated cytotoxic antineoplastic therapy in cancer patients.

No therapeutic agent and no therapy for GVHD wherein the GVHD is treated by inhibiting Fas-mediated apoptosis are known to date. In addition, no therapeutic agent and no therapy for GVHD wherein the GVHD is treated by utilizing selective immunosuppression are known to date.

With regards to diseases based on ischemic reperfusion injury, commercially available drugs mainly aim at thrombolysis, and improvement of circulation, and no drug is available that directly prevents or treats the damage.

The drugs used for the diseases based on organ damage mainly aim at palliative treatment, and no drug is available that prevents or radically treats the diseases based on organ damage. In addition, no prophylactic or therapeutic agent which is widely effective for various tissues and organs is available.

Treatment in accordance with the present invention may be used to provide clear benefit for patients. Treatment may be given by injection (e.g. intravenously) or by local delivery methods (e.g. pre-coating of stents or other indwelling devices). The antibody molecule may be administered via any suitable route, including for example systemically, e.g. intraperitoneally, or intravenously, or locally, e.g. intrathecally or by lumbar puncture. Intravenously administration may be preferred, except for the treatment of neurological disorders and injuries where intrathecally administration may be preferred.

Anti-Fas may be delivered by gene-mediated technologies. Alternative formulation strategies may provide preparations suitable for oral or suppository route. The route of administration may be determined by the physicochemical characteristics of the treatment, by special considerations for the disease, to optimise efficacy or to minimise side-effects.

In accordance with the present invention, compositions provided may be administered to individuals. Administration is preferably in a “therapeutically effective amount”, this being sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage etc, is within the responsibility of general practitioners and other medical doctors. Appropriate doses of antibody are well known in the art; see Ledermann J. A. et al. (1991) Int. J. Cancer 47: 659-664; Bagshawe K. D. et al. (1991) Antibody, Immunoconjugates and Radiopharmaceuticals 4: 915-922.

The precise dose will depend upon a number of factors, including whether the antibody is for diagnosis or for treatment, the size and location of the area to be treated, the precise nature of the antibody (e.g. whole antibody, fragment or diabody), and the nature of any detectable label or other molecule attached to the antibody. A typical antibody dose will be in the range 0.5 mg-1.0 g, and this may be administered as a bolus intravenously. Other modes of administration include intravenous infusion over several hours, to achieve a similar total cumulative dose. This is a dose for a single treatment of an adult patient, which may be proportionally adjusted for children and infants, and also adjusted for other antibody formats in proportion to molecular weight. Treatments may be repeated at daily, twice-weekly, weekly or monthly intervals, at the discretion of the physician.

A further mode of administration employs precoating of, or otherwise incorporation into, indwelling devices, for which the optimal amount of antibody will be determined by means of appropriate experiments.

Binding members of the present invention will usually be administered in the form of a pharmaceutical composition, which may comprise at least one component in addition to the binding member.

Thus pharmaceutical compositions according to the present invention, and for use in accordance with the present invention, may comprise, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

A composition may include a stent or other indwelling device.

A composition may be administered alone or in combination with other treatments, either simultaneously or sequentially dependent upon the condition to be treated. Other treatments may include the administration of suitable doses of pain relief drugs such as non-steroidal anti-inflammatory drugs (e.g. asprin, paracetamol, ibuprofen or ketoprofen) or opiates such as morphine, or anti-emetics.

A composition in accordance with the present invention may be administered in a case of acute disease or injury. Treatment may be started as soon as possible, e.g. immediately after the occurrence of organ or tissue injury, or detection of ischemia. The composition may be administered once or more than once.

Another aspect of the present invention provides as a preservative for an organ such as heart, kidney, liver or islets characterized by its inclusion of a Fas antagonist of the invention as its effective component.

The present invention provides a method comprising causing or allowing binding of a binding member as provided herein to Fas. As noted, such binding may take place in vivo, e.g. following administration of a binding member, or nucleic acid encoding a binding member, or it may take place in vitro, for example in ELISA, Western blotting, immunocytochemistry, immuno-precipitation or affinity chromatography.

The amount of binding of binding member to Fas may be determined. Quantitation may be related to the amount of the antigen in a test sample, which may be of diagnostic interest.

The reactivity of antibodies on a sample may be determined by any appropriate means. Radioimmunoassay (RIA) is one possibility. Radioactive labelled antigen is mixed with unlabelled antigen (the test sample) and allowed to bind to the antibody. Bound antigen is physically separated from unbound antigen and the amount of radioactive antigen bound to the antibody determined. The more antigen there is in the test sample the less radioactive antigen will bind to the antibody. A competitive binding assay may also be used with non-radioactive antigen, using antigen or an analogue linked to a reporter molecule. The reporter molecule may be a fluorochrome, phosphor or laser dye with spectrally isolated absorption or emission characteristics. Suitable fluorochromes include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed.

The signals generated by individual antibody-reporter conjugates may be used to derive quantifiable absolute or relative data of the relevant antibody binding in samples (normal and test).

The present invention also provides the use of a binding member as above for measuring antigen levels in a competition assay, that is to say a method of measuring the level of antigen in a sample by employing a binding member as provided by the present invention in a competition assay. This may be where the physical separation of bound from unbound antigen is not required. Linking a reporter molecule to the binding member so that a physical or optical change occurs on binding is one possibility. The reporter molecule may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule.

The present invention also provides for measuring levels of antigen directly, by employing a binding member according to the invention for example in a biosensor system.

The mode of determining binding is not a feature of the present invention and those skilled in the art are able to choose a suitable mode according to their preference and general knowledge.

The present invention further extends to a binding member which competes for binding to Fas with any binding member which both binds the antigen and comprises a V domain including a CDR with amino acid substantially as set out herein or a V domain with amino acid sequence substantially as set out herein. Competition between binding members may be assayed easily in vitro, for example by tagging a specific reporter molecule to one binding member which can be detected in the presence of other untagged binding member(s), to enable identification of binding members which bind the same epitope or an overlapping epitope. Competition may be determined for example using ELISA or flow cytometry.

A competition reaction may be used to select one or more binding members such as derivatives of F45D9, which may have one or more additional or improved properties.

In testing for competition a peptide fragment of the antigen may be employed, especially a peptide including an epitope of interest. A peptide having the epitope sequence plus one or more amino acids at either end may be used. Such a peptide may be said to “consist essentially” of the specified sequence. Binding members according to the present invention may be such that their binding for antigen is inhibited by a peptide with or including the sequence given. In testing for this, a peptide with either sequence plus one or more amino acids may be used.

Binding members which bind a specific peptide may be isolated for example from a phage display library by panning with the peptide(s).

The present invention further provides an isolated nucleic acid encoding a binding member of the present invention. Nucleic acid includes DNA and RNA. In a preferred aspect, the present invention provides a nucleic acid which codes for a CDR or VH or VL domain of the invention as defined above.

The present invention also provides constructs in the form of plasmids, vectors, transcription or expression cassettes which comprise at least one polynucleotide as above.

The present invention also provides a recombinant host cell which comprises one or more constructs as above. A nucleic acid encoding any CDR, VH or VL domain, or binding member as provided itself forms an aspect of the present invention, as does a method of production of the encoded product, which method comprises expression from encoding nucleic acid. Expression may conveniently be achieved by culturing under appropriate conditions recombinant host cells containing the nucleic acid. Following production by expression a VH or VL domain, or binding member may be isolated and/or purified using any suitable technique, then used as appropriate.

Binding members, VH and/or VL domains, and encoding nucleic acid molecules and vectors according to the present invention may be provided isolated and/or purified, e.g. from their natural environment, in substantially pure or homogeneous form, or, in the case of nucleic acid, free or substantially free of nucleic acid or genes origin other than the sequence encoding a polypeptide with the required function. Nucleic acid according to the present invention may comprise DNA or RNA and may be wholly or partially synthetic. Reference to a nucleotide sequence as set out herein encompasses a DNA molecule with the specified sequence, and encompasses a RNA molecule with the specified sequence in which U is substituted for T, unless context requires otherwise.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, NS0 mouse melanoma cells, YB2/0 rat myeloma cells and many others. A common, preferred bacterial host is E. coli.

The expression of antibodies and antibody fragments in prokaryotic cells such as E. coli is well established in the art. For a review, see for example Plückthun, A. Bio/Technology 9: 545-551 (1991). Expression in eukaryotic cells in culture is also available to those skilled in the art as an option for production of a binding member, see for recent reviews, for example Ref, M. E. (1993) Curr. Opinion Biotech. 4: 573-576; Trill J. J. et al. (1995) Curr. Opinion Biotech 6: 553-560.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. ‘phage, or phagemid, as appropriate. For further details see, for example, Sambrook and Russell, Molecular Cloning: a Laboratory Manual: 3rd edition, 2001, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Ausubel et al. eds., Short Protocols in Molecular Biology: A Compendium of Methods from Current Protocols in Molecular Biology, John Wiley & Sons, 4^{th} edition 1999. The disclosures of Sambrook et al. and Ausubel et al. are incorporated herein by reference.

Thus, a further aspect of the present invention provides a host cell containing nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage.

The introduction may be followed by causing or allowing expression from the nucleic acid, e.g. by culturing host cells under conditions for expression of the gene.

In one embodiment, the nucleic acid of the invention is integrated into the genome (e.g. chromosome) of the host cell. Integration may be promoted by inclusion of sequences which promote recombination with the genome, in accordance with standard techniques.

The present invention also provides a method which comprises using a construct as stated above in an expression system in order to express a binding member or polypeptide as above.

Aspects and embodiments of the present invention will now be illustrated by way of example with reference to the following experimentation, without limitation to the scope of the invention.

All documents cited in this specification are incorporated by reference.

Example 1: Generation of the human anti-Fas F45D9 monoclonal antibody.
Example 2: F45D9-γ1 and F45D9-γ4 mAbs binds to the surface of Fas expressing human T cells.
Example 3: F45D9-γ1 and F45D9-γ4 mAb binds Fas molecule in a specific manner.
Example 4: Fas binding affinity of F45D9-γ1 and F45D9-γ4 mAb.
Example 5: Epitope mapping of F45D9-γ1 mAb.
Example 6: In vitro antagonistic activity of F45D9-γ1 and F45D9-γ4 mAbs, as wells as F(ab)₂ and Fab fragments of F45D9-γ1 mAb in rhFasL-induced apoptosis in Human T and B cells.
Example 7: In vitro antagonistic activity of F45D9-γ1 and F45D9-γ4 mAb in Activation Induced Cell Death (AICD) in Human T cells.
Example 8: In vivo antagonistic activity of F45D9-γ1 mAb in FasL-induced cell death (SCID mice model).
Example 9: Reactivity of F45D9-γ1 and F45D9-γ4 with Fas molecules of various species, non-human primate common marmoset and chimpanzee. Reactivity of F45D9-γ1 and F45D9-γ4 mAbs with Fas molecules on marmoset PBMC and B cell lines. Comparison of binding affinity between marmoset and human lymphocytes. Immunohistochemical cross-reactivity study with F45D9-γ4 mAb in human and marmoset tissues.
Example 10: In vitro antagonistic activity of F45D9-γ1 and F45D9-γ4 mAb in rhFasL-induced apoptosis in B cells and activated lymphocytes from non-human primate common marmoset.
Example 11: F45D9-γ1-mediated co-stimulatory signal in activation and proliferation of human T cells.
Example 12: Effect of F45D9-γ1 and F45D9-γ4 mAbs in inducing antibody dependent cell mediated cytotoxicity (ADCC).
Example 13: Effect of F45D9-γ1 mAb in inducing Complement Dependent Cytotoxicity (CDC).
Example 14: Toxicity test of F45D9-γ1 mAb, in primary human hepatocytes.
Example 15: Pilot toxicity study in marmosets with F45D9 mAbs.
Example 16: Effect of the human F45D9 anti-Fas antibody in skin explants model of human GVHD.
Example 17: Effect of F45D9-γ4 mAb on Human cytotoxic T cell (CTL) activity in vitro

### EXAMPLE 1

Generation of the Human Anti-Fas F45D9 Monoclonal Antibody

The anti-human Fas monoclonal antibody (F45D9/1F8/6) that led to the subsequent development of the recombinant human Mab T17/B2-1G4 (gamma-1 isotype) and Mab T19/B5-1F9 (gamma-4 isotype) was generated by hybridoma antibody technique using two (2) HuMAb transgenic mice (two different batches: batch 2 and batch 3) from Medarex Inc. (Cottonwood Drive, Calif., USA) that were immunized at Microbiology and Tumorbiology Center (MTC) laboratories and animal facilities at Nobels väg 16, Stockholm, Sweden (the immunization schedule shown in the Table 1). The genotypes of the mice were: Mouse 3(3^{rd} batch) Genotype I [(CMD)++; (HCo12)15087+; (JKD)++; (KCo5)9272+] and Mouse 18(2^{nd} batch) Genotype II [(CMD)++; (HCo12)15087+; (HCo7)11952; (JKD)++; (KCo5)9272+]. Each mouse comprises disrupted mouse heavy and mouse kappa light chain loci, designed CMD and JKD, respectively. These disruptions prevent the expression of any antibodies that are completely murine. Nevertheless, they still allow for the expression of two different types of mouse immunoglobulins sequences. Mouse non-mu heavy chain isotype sequences are expressed as components of chimeric human/mouse heavy chains, and mouse lambda light chains are expressed as hybrid human/mouse antibodies.

The transgenic Mouse 18 (2^{nd} batch) was immunized by intraperitoneal injection (i.p.) with whole Jurkat cells (Human T cell leukemia cell line, DSMZ ACC 282) expressing human Fas surface receptor (10⁷ live cells/mouse in PBS) combined with 10 ug/mouse of recombinant human soluble Fas (recombinant human soluble Fas obtained from PeproTech EC Ltd., London W6 8LL, UK, Cat No. 310-20) or rhFas/Fc chimera (recombinant human (NSO-derived) from R&D Systems, Minneapolis, Minn. 55413, USA; Cat. No. 326-FS/CF). The 4^{th}-7^{th} immunizations were done with 5 or 10 ug/mouse of recombinant human soluble Fas (from PeproTech EC Ltd., London W6 8LL, UK) or rhFas/Fc chimera (from R&D Systems, Minneapolis, Minn. 55413, USA) plus 10 ug/mouse of peptides FP5, FP8, FP9, FP11, FP18 (sequences in U.S. Pat. No. 6,846,637 B1) (Peptide FP5-KLH, SigmaGenosys, #94519-1; Peptide FP9-KLH, SigmaGenosys, #96221-1; Peptide FP11-KLH, SigmaGenosys, #94519-3; Peptide FP8-KLH (peptide 3-KLH, Thermo Hybaid,); Peptide FP18-KLH (peptide 5-KLH, Thermo Hybaid, 354/3) together with adjuvant RIBI MPL+TDM Emulsion (SIGMA, M-6536). Three boosts were done by intravenously injection (i.v.) with 10 ug/mouse of recombinant human sFas (PeproTech EC Ltd) and non-conjugated peptides FP5, FP8, FP9, FP11, FP18.

The transgenic Mouse 3(3^{rd} batch) was immunized by intraperitoneal injection (i.p.) with 10 or 20 ug/mouse of recombinant human soluble Fas (from PeproTech EC Ltd., London W6 8LL, UK, Cat No. 310-20) together with adjuvant RIBI MPL+TDM Emulsion (SIGMA, M-6536). The 3^{th} and 5^{th} immunizations were done with 10 ug/mouse of recombinant human soluble Fas (from PeproTech EC Ltd) plus 10 ug/mouse of peptides FP5, FP8, FP9, FP11, FP18, together with adjuvant RIBI MPL+TDM Emulsion (SIGMA, M-6536). Three boosts were done by intravenously injection (i.v.) with 10 ug/mouse of recombinant human sFas (PeproTech EC Ltd) and non-conjugated peptides FP5, FP8, FP9, FP11, FP18.

After the immunization and confirmation of the increase of the serum level of the desired anti-Fas antibody (by ELISA), the splenocytes were isolated from the animals and subjected to cell fusion. The spleen cells from the two mice (mouse 18 and 3) were used for the fusion. Spleen cells were fused with the myeloma cell line Sp2/0 cells (European Collection of Cell Cultures (ECACC) at a ratio 2:1, in the presence of PEG and RPMI-1640 medium, followed by culture in 20% FBS/RPMI/OPI/HAT. The parent cell to be fused with the splenocytes is not limited to any particular type, however Sp2/0 myeloma cell line is preferred as a fusion partner.

The hybridomas were then screened for Fas binding by ELISA for the one producing the target antibody used in the present invention and subsequently cloned. The clone F45D9 was selected for further development based on the data of ELISA screening; cell growth feature; and FACS analysis. Supernatant from F45D9 hybridoma was able to bind to recombinant human soluble Fas (PeproTech EC Ltd) on ELISA (see method below), and bind to Jurkat cells expressing human Fas on cell surface (FACS analysis according EXAMPLE 2), and to inhibit FasL-induced apoptosis in Jurkat cells without inducing apoptosis itself (Apoptosis assay according EXAMPLE 6).

ELISA for screening of positive cell clones after fusion: Microtiter plate wells were coated overnight at 4° C. with 25 μl/well of 0.56 ug/ml (14 ng/well) of Human recombinant soluble Fas (PeproTech EC Ltd., London W6 8LL, UK, Cat No. 310-20) diluted in coating buffer (Sodium carbonate buffer 0.1M; pH 9.6). The wells were then emptied and blocked with 2% Ovalbumin in PBS at room temperature (RT) for 1 hr. After washing three times with washing buffer (0.02M Tris; NaCL 0.15M; 0.05% Tween 20) 40 ul of culture medium from wells containing hybridoma cells was added to each well for 90 minutes at RT. The wells were then washed six times with washing buffer and peroxidise-conjugated rabbit anti-human IgG (Dako, Cat. P0214) or peroxidise-conjugated rabbit anti-human kappa light chain (Dako, Cat. P0219) antibodies were added and incubated for 1 h at RT. After washing substrate buffer containing 3,5-tetramethylbenzidine (BD Opt EIA™ Substrate, BD Biosciences PharMingen) were added. Reaction was stopped after 30 min by the addition of 1M H₂SO₄ and absorbance at 450 nm was measured.

The F45D9 clone was subcloned by limit dilution and the subclones were further screened for binding to recombinant human sFas in ELISA (described before). The subclone 1F8 (F45D9/1F8) was chosen for further development. The clone was expanded, adapted into serum free & protein free CD medium (Gibco, 11279-023), and frozen for safety bank storage. The F45D9/1F8 clone was re-cloned by limit dilution to ensure the monoclonality. The F45D9/1F8 subclones were screened by ELISA to detect fully human anti-Fas antibodies (binding to recombinant human sFas) using peroxidase-conjugated goat anti-human IgG Fcγ fragment specific antibody (Jackson ImmunoResearch, cat. 109-035-098) instead of the rabbit anti-human IgG (Dako, Cat. P0214) antibodies used in previous ELISA; or peroxidase-conjugated rabbit anti-human kappa light chain (Dako, Cat. P0219); or peroxidase-conjugated goat anti-mouse IgG Fcγ fragment specific antibody (Jackson ImmunoResearch, cat. 115-035-071). The screening test with these goat antibodies showed reactivity of F45D9 antibody with anti-mouse IgG Fcγ fragment specific antibody and not with anti-human IgG Fcγ fragment specific antibody, indicating that the antibody probably contains chimeric human/mouse IgG heavy chains. The F45D9 antibody also showed reactivity with anti-human kappa light chain specific antibody. After screening the subclone “6” (F45D9/1F8/6) was chosen for further development to generate fully human antibody against human Fas. The clone was expanded, adapted into serum free & protein free CD medium, and frozen for safety bank storage.

### Generation of Recombinant Human mAb T17/B2-1G4 (Gamma-1 Isotype)

cDNA was cloned and sequenced for the variable regions of the heavy and light chains of the F45D9/1F8/6 clone. Three (3) sequence patterns (from 11 clones) were obtained for VL, and one VH sequence was observed from 3 clones. The work employed an RT protocol as well as a 5′RACE protocol. The cDNA sequence of the variable regions' fragments of the heavy and light chains of the F45D9/1F8/6 antibody were checked with GenBank, and the variable regions of the heavy and light chains of the antibody were as human origin. The constant regions of the antibody matched mouse immunoglobulin sequences.

The fragments of variable regions of the heavy and light chains of the F45D9/1F8/6 antibody were cloned into pCR®2.1-TOPO® vector (Invitrogen, cat. K4500) and transformed into E. coli (One Shot® competent cells, Invitrogen, cat. 44-0301). In order to obtain human antibody, the fragments of the heavy and light chains' variable regions were re-cloned into the pIESRαγ1fa vector from Medarex that contains SR alpha promoters and constant regions of human kappa light chain and human gamma1 heavy chain that is γ1fa allotype.

PCR was performed for adding restriction sites to VL (kappa light chain variable region) fragment for pIE vector insertion. VL-RACE-7 fragment was amplified by PCR using RACE-7_F and VL-RACE-7_R primers. The PCR fragments were re-cloned back to pCR®2.1-TOPO® vector (Invitrogen cat. 46-0801) in order to gain enough material for further work. The correct VL fragment was cleaved with Bg1 II/Bsi WI enzymes from purified VL-RACE-7-B plasmid DNA and inserted into pIESRαγ1fa-(KV#1/VH-S1) vector (digested with Bg1 II/Bsi WI enzymes) with T4 DNA ligase (Invitrogen, Cat. 15224-017). The pIESRαγ1fa-(KV#1/VH-S1) vector was reconstructed by inserting VH-S1 segments into pIESRαγ1fa vector. The correct insertion into the vector was checked by PCR using pIE-F and PIE-KV-R primers. The reconstructed vector was named: VL-RACE-7-pIESRαγ1fa(VH-S1). In order to gain enough material for transfection, as well as for back up storage MAX Efficiency Stb12 competent cells (Invitrogen Cat. #10268-019) were transformed with the VL/VH inserted pIESRαγ1fa vector (VL-RACE-7-pIESRαγ1fa(VH-S1).

The reconstructed plasmid (VL-RACE-7-pIESRαγ1fa(VH-S1) was transfected into CHO DG44 cells (obtained directly from Professor Lawrence Chasin, Columbia University, MC2433, New York, N.Y. 10027, USA) with Lipofectamine™ 2000 (Invitrogen, cat. 11668-027) in 24-well plates. ELISA was applied to screen the transfected CHO DG44 clones. The ELISA procedure was same as before for detecting human anti-human Fas antibodies using Recombinant human soluble Fas obtained from PeproTech EC Ltd and rabbit anti-Human kappa light chain-HRP (DAKO, cat P0129). Another ELISA was applied to detect whole human IgG Immunoglobulin, by coating with goat anti-human IgG F(ab)₂ fragment specific (Jackson ImmunoResearch, cat. 109-005-097) and detecting with secondary antibody peroxidase-conjugated goat anti-human IgG Fcγ fragment specific (Jackson ImmunoResearch, cat. 109-035-098). Supernatants from the clone T17/B2 (T17/L18/B2) showed positive to rFas in ELISA and showed to be whole human kappa/IgG. The antibody's prospective biological activity was also confirmed: supernatant from T17/B2 (T17/L18/B2) clone was able to inhibit FasL-induced apoptosis in Jurkat cells (method in EXAMPLE 6).

The T17/B2 (T17/L18/B2) clone was subcloned by limit dilution in 3×96-well plates. Twenty-nine (29) T17/B2 (T17/L18/B2) subclones were screened by the described ELISA and the subclone “T17/L18/B2-1G4” was chosen for further development based on the data of ELISA screening; cell growth feature; and biological activity. Supernatant from subclone “T17/L18/B2-1G4” was able to inhibit FasL-induced apoptosis in Jurkat cells and not to induce apoptosis by itself. The subclone “T17/L18/B2-1G4” was cultivated in F-12 (Ham) medium (Gibco, cat. 31765-027) with FBS, following adaptation to chemically defined, protein-free medium—CD DG44 Medium. While adaptation the T17/L18/B2-1G4 clone has been cultivated 5 passages in 5% FCS-F-12 medium; 5 passages in 2.5% FCS-F-12 medium; and 9 passages in 1.25% FCS-F-12 medium; and eventually in 100% CD DG44 Medium. Cells adapted to 100% CD DG44 medium were growing well, and were producing antibody with good biological activity.

### Generation of Recombinant Human mAb T19/B5-1F9 (Gamma-4 Isotype):

In order to obtain the recombinant human T19/B5-1F9 (gamma-4 isotype) antibody, the fragments of the heavy and light chains' variable regions from T17/L18/B2-1G4 clone were re-cloned into the pIESRαγ4P vector from Medarex that is containing SR alpha promoters and constant regions of human kappa light chain and human gamma4 heavy chain that is γ4P allotype.

The correct VH fragment (VH-S1) was cleaved with Nhe I/Not I enzymes from purified VL-RACE-7-pIESRαγ1fa(VH-S1) plasmid DNA and inserted into pIESRαγ4P vector (digested with Nhe I/Not I enzymes) with T4 DNA ligase (Invitrogen, Cat. 15224-017). The reconstructed vector was named: pIESRαγ4P-VH-S1. In order to gain enough material for further work, as well as for back up storage, MAX Efficiency Stb12 competent cells (Invitrogen Cat. #10268-019) were transformed with the pIESRαγ4P-VH-S1 vector. The correct insertion into the vector was checked by PCR using pIE-F and VH_R primers. The correct VL fragment (VL-RACE-7) was cleaved with Bg1 II/Bsi WI enzymes from purified VL-RACE-7-pIESRαγ1fa(VH-S1) plasmid DNA and inserted into pIESRαγ4P-VH-S1 vector (digested with Bg1 II/Bsi WI enzymes) with T4 DNA ligase (Invitrogen, Cat. 15224-017). The correct insertion into the vector was checked by PCR using pIE-KV_F and pIE-KV_R primers. The reconstructed vector was named: VL-RACE-7-pIESRαγ4P (VH-S1). In order to gain enough material for transfection, as well as for back up storage MAX Efficiency Stb12 competent cells (Invitrogen Cat. #10268-019) were transformed with the VL/VH inserted pIESRαγ4P vector (VL-RACE-7-pIESRαγ4P(VH-S1).

The reconstructed plasmid (VL-RACE-7-pIESRαγ4P(VH-S1) was transfected into CHO DG44 cells with Lipofectamine™ 2000 (Invitrogen, cat. 11668-027). ELISA was applied to screen the transfected cells. The ELISA procedure was same as before for detecting human anti-human Fas antibodies using Recombinant human soluble Fas obtained from PeproTech EC Ltd and Rabbit anti-Human kappa light chain-HRP (DAKO, cat P0129). Another ELISA was applied to detect whole human IgG Immunoglobulin, by coating with goat anti-human IgG F(ab)₂ fragment specific (Jackson ImmunoResearch, cat. 109-005-097) and detecting with secondary antibody peroxidase-conjugated goat [F(ab′)2 fragment] of anti-human IgG Fcγ fragment specific (Jackson ImmunoResearch, cat. 109-036-098). Supernatants from the clone T19/B5 (T19/L22/B5) showed positive to rFas in ELISA and showed to be whole human kappa/IgG. IgG4 isotype of the T19/B5 (T19/L22/B5) clone was also confirmed by ELISA, by coating with sheep anti-human IgG4 specific (THE BINDING SITE, cat. PC009) and detecting with secondary antibody Rabbit anti-Human kappa light chain-HRP (DAKO, cat P0129). The antibody's prospective biological activity was also confirmed: supernatant from T19/B5 (T19/L22/B5) clone was able to inhibit FasL-induced apoptosis in Jurkat cells (see method in EXAMPLE 6).

The T19/B5 (T19/L22/B5) clone was subcloned by limit dilution in 2×96-well plates. Eighteen (18) T19/B5 (T19/L22/B5) subclones were screened by the described ELISA. The subclone “T19/L22/B5-1F9” was chosen for further development based on the data of ELISA screening, cell growth feature and biological activity. Supernatant from subclone “T19/L22/B5-1F9” was able to bind to Jurkat (from ATCC, Jurkat, clone E6-1, ATCC-TIB-152, Human T Leukemia cell line) and SKW6.4 cells (from ATCC, ATCC-TIB-215, Human B lymphoblastoid cell line), both expressing human Fas on cell surface, and to inhibit FasL-induced apoptosis in SKW6.4 cells and not to induce apoptosis by itself in these cells (see method in EXAMPLE 2 and 6, respectively). The T19/L22/B5-1F9 clone was cultivated in F-12 (Ham) medium (Gibco, cat. 31765-027) with FBS. The cells were adapted to chemically defined, protein-free medium—CD DG44 Medium (Gibco, Cat. 12610-010). While adaptation the T19/L22/B5-1F9 clone has been cultivated 3 passages in 2.5% FCS-F-12 medium; 7 passages in 1.25% FCS-F-12 medium; and 3 passages in 0.3% FCS-F-12 medium; and eventually in 100% CD DG44 Medium. The cells were growing well, and have been producing antibody with good biological activity.

### EXAMPLE 2

### F45D9-γ1 and F45D9-74 mAb Binds to the Surface of Fas Expressing Human T Cells

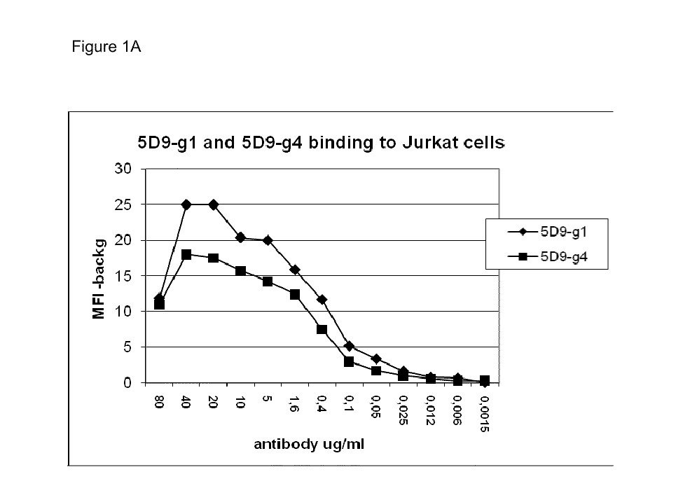

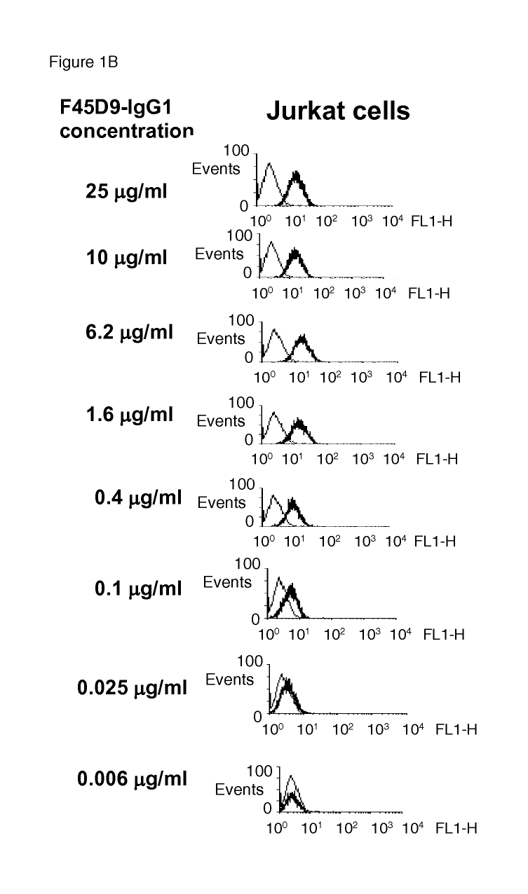

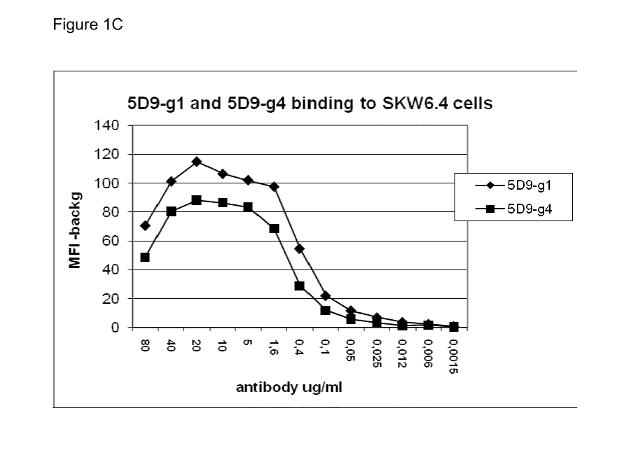

Binding of F45D9-γ1 and F45D9-γ4 to the surface of Fas expressing Jurkat and SKW6.4 cells (from ATCC, Jurkat, clone E6-1, ATCC-TIB-152, Human T Leukemia cell line); SKW6.4 cells, ATCC-TIB-215, Human B lymphoblastoid cell line) was explored by immunofluorescence staining and flow cytometry analysis. Cells were cultured in RPMI 1640 medium (GIBCO, Cat. 3105205) supplemented with 2 mM glutamax (GIBCO), 100 UI/mL penicillin, 100 μg/mL streptomycin (Sigma) and 5-10% fetal bovine serum (GIBCO), at 37° C. and 5% CO₂. After washing in PBS Jurkat or SKW6.4 cells (0.2×10⁶ cells/well) were incubated in staining buffer (PBS/1% FBS) containing F45D9-γ1, F45D9-γ4 or human IgG control antibodies (Human IgG1, kappa, Sigma, Cat. I5154 or Human IgG4, kappa, Sigma, Cat. I4639) in a 96-well U-bottom plate for 30 min on ice, in a 100 ul/well volume. Cells were then washed once with staining buffer, and further incubated for 30 min on ice with 1:30 dilution of FITC-conjugated rabbit F(ab′)₂ anti-human IgG (DakoCytomation Cat. F0056). After washing and fixing with 1% formaldehyde in PBS cells were analyzed in a FACScan (Becton Dickinson, Mountain View, Calif.). The reactivity of F45D9-γ1 and F45D9-γ4 to the surface of Jurkat cells was quantified and Geometric Mean Fluorescence Intensity (MFI) after background subtraction (FIG. 1A) and histograms (FIG. 1B) are shown. The bold solid line indicates staining by F45D9-γ1 mAb and the light solid line represents staining by isotype control antibodies. FIG. 1C shows binding of F45D9-γ1 and F45D9-γ4 mAb to the surface of SKW6.4 cells; results are shown as Geometric Mean Fluorescence Intensity (MFI) after background subtraction. Forward and side scatter gates were set to exclude dead cells. Data from a representative of two experiments are shown.

### EXAMPLE 3

### F45D9-γ1 and F45D9-γ4 mAb Binds Fas Molecule in a Specific Manner

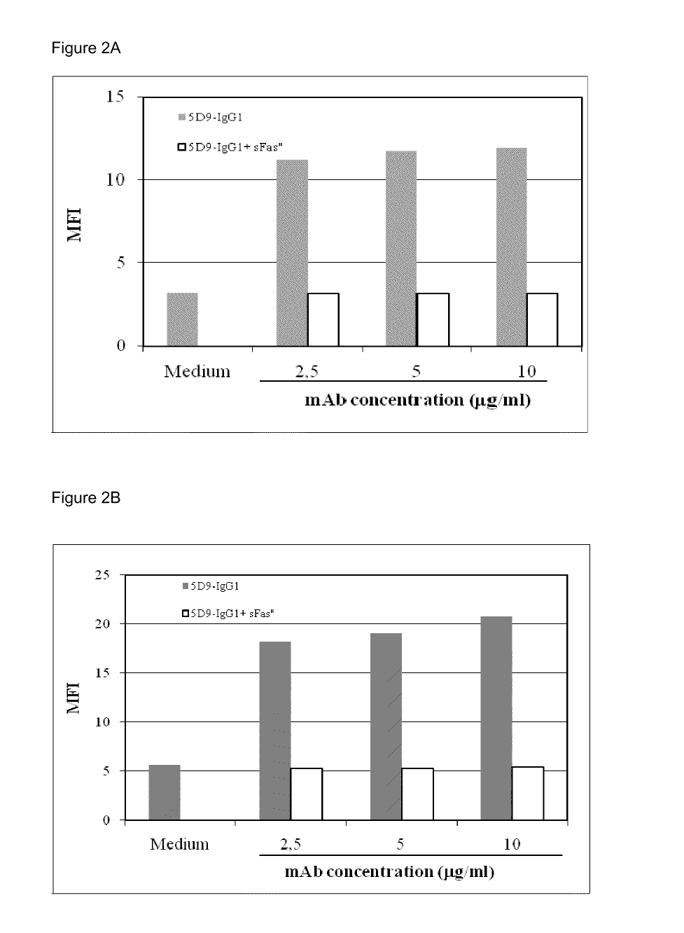

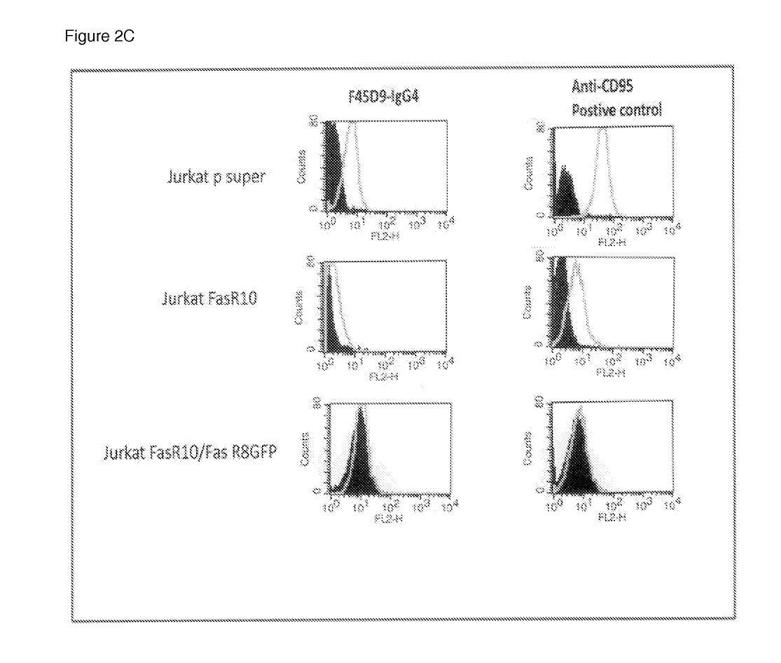

F45D9-γ1 mAbs, diluted in staining buffer (PBS/1% FBS), were pre-incubated or not in 96-well U-bottom plates during 1 h with 40 μg/ml of recombinant sFas (recombinant human soluble Fas receptor, Peprotech EC Ltd, Cat. 310-20). Then 0.2×10⁶ (FIG. 2A) Jurkat (FIG. 2B) SKW6.4 cells (malignant human lymphoblastoid B cell), were added per well and plate was incubated for 30 min on ice. Cells were then washed once with staining buffer, further incubated for 30 min on ice with 1:30 dilution of FITC-conjugated rabbit F(ab′)2 anti-human kappa (DakoCytomation Cat. F0434). After washing and fixing with 1% formaldehyde in PBS cells were analyzed in a FACScan (Becton Dickinson, Mountain View, Calif.). Forward and side scatter gates were set to exclude dead cells. Data represent specific mean florescence intensity (MFI) after subtraction of background MFI (using human IgG control antibodies (Human IgG1, kappa, Sigma, Cat. I5154)). FIGS. 2A and 2B shows data from a representative of two experiments are shown.

Binding of F45D9-γ4 to the surface of different Jurkat cell lines expressing different levels of Fas after transduction with anti-Fas siRNA to knock-down Fas expression (Dotti G., et al, Blood, 105:4677-4684, 2005) was explored by immunofluorescence staining and flow cytometry analysis. Jurkat p super (transduced with empty vector), Jurkat FasR10 (transduced with anti-Fas siRNA10) and Jurkat FasR10/FasR8GFP (sequentially transduced with anti-Fas siRNA10 and GFP-siRNA8) were obtained from Dr. G. Dotti Laboratory (Center for Cell and Gene Therapy; Baylor College of Medicine, Houston, Tex., USA) and were cultured in RPMI 1640 medium (GIBCO, Cat. 3105205) supplemented with 2 mM glutamax (GIBCO), 100 UI/mL penicillin, 100 μg/mL streptomycin (Sigma) and 5-10% fetal bovine serum (GIBCO), at 37° C. and 5% CO₂. After washing in PBS Jurkat cells were incubated in staining buffer (PBS/1% FBS) containing 10 ug/ml of F45D9-γ4 in a 96-well U-bottom plate for 30 min on ice, in a 100 ul/well volume. Cells were then washed once with staining buffer, and further incubated for 30 min on ice with 1:30 dilution of PE-conjugated rabbit F(ab′)₂ anti-human kappa (DakoCytomation Cat. F0436). As positive control PE-labelled mouse anti-CD95 mAb (BD Pharmingen Cat. 555674) was used. After washing and fixing with 1% formaldehyde in PBS, cells were analyzed in a FACScan as described before. FIG. 2C shows histograms of F45D9-γ4 mAb binding to the surface of Jurkat cells expressing different levels of Fas. The bold solid line indicates staining by F45D9-γ4 mAb or anti-CD95 positive control mAb and filled histogram represents staining with control antibodies.

### Results

FIGS. 2A and 2B shows that pre-incubation of F45D9-γ1 mAbs with recombinant sFas completely blocked the binding of the antibodies to the surface of Fas expressing Jurkat or SKW6.4 cell lines.

FIG. 2C shows no binding of F45D9-γ4 mAb to Jurkat cells with complete knock-down expression of Fas (Jurkat FasR10/FasR8GFP), demonstrating the specific binding of F45D9-γ4 mAb to Fas molecule.

### EXAMPLE 4

### Fas Binding Affinity of F45D9-γ1 and F45D9-γ4 mAb

The interaction of F45D9 mAb's (γ-γ1 and γ4-isotypes) with immobilized soluble human Fas receptor was monitored by surface plasmon resonance detection using a BIAcore 3000 instrument. Recombinant human soluble Fas receptor (srFas) (PeproTech, cat. 310-20) was immobilized (concentration of 5 ug/ml in immobilization buffer: 10 mM sodium acetate pH 5.0) onto a CM5 sensor chip (BIAcore BR-1001-14) using an Amine Coupling kit (BIAcore, Cat. BR-1000-50), at a surface density of 1000110 resonance units (RU). Deactivation of excess reactive groups on the chip surface was done by adding 1.0 M ethalonamine hydrochloride (pH 8.5). F45D9 mAb's-γ1 mAb were passed over the surface in equilibrium binding experiments at concentrations ranging from 2.123 to 68132 nM at a flow rate of 30 ul/min. Dilutions and binding experiments were conducted in 0.01 M HEPES (pH 7.4), 0.15 M NaCl, 3 mM EDTA, and 0.005% P-20 (BIAcore surfactant, BR-1001-88). Between each cycle 100 mM HCl was used to regenerate the surfaces at a flow rate 30 ul/min. K_{A} and K_{D} of F45D9 mAb's were-γ1 mAb was determined by fitting bivalentusing BIAcore models (BIAevaluation).

### Results

The calculated binding constant K_{D} for IgG1 is 4.11×10⁻¹¹ M, Chi values 139; K_{D} for IgG4 is 5.87×10⁻¹¹ M, Chi values 51.

Values obtained from BIAcore binding affinity analysis are: K_{A} 6.5×10⁹ M⁻¹ and K_{D}1.53×10⁻¹⁰ M, Chi values 0.75.

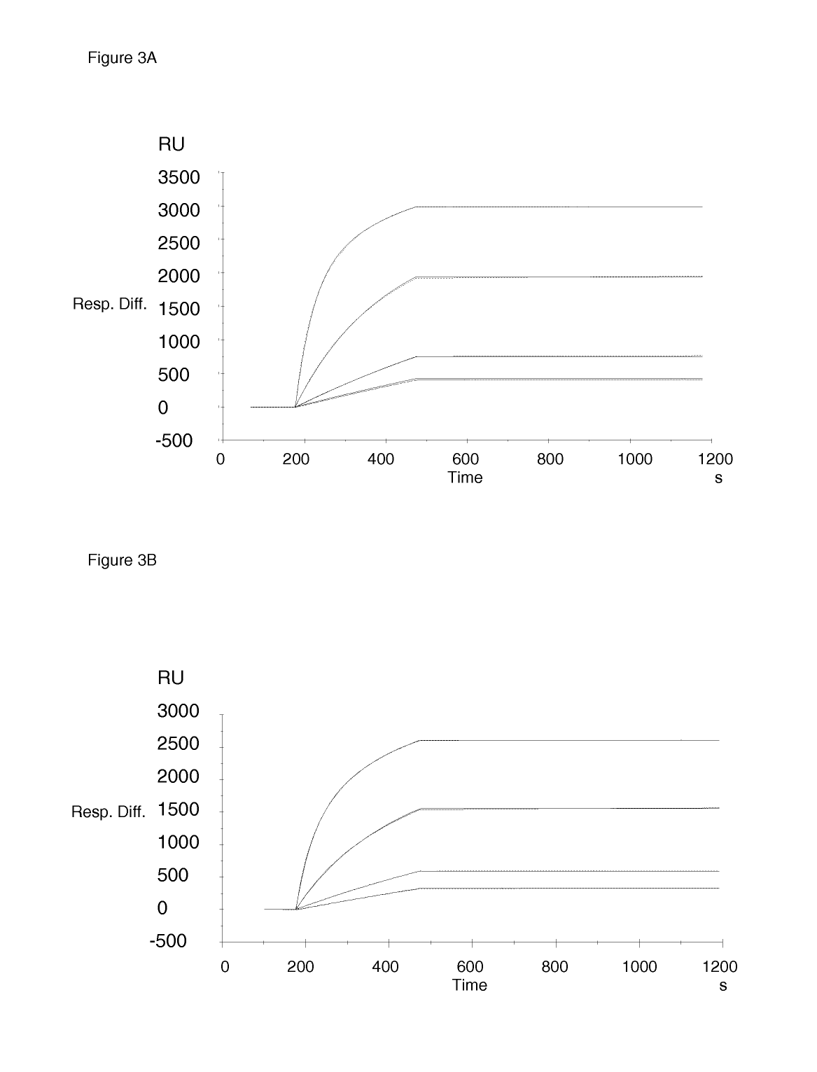

FIG. 3A shows sensograms and bivalent analyte fit of Fas/IgG1 interaction data (superimposed). Coloured lines are injections of IgG1 at different showing the binding of F45D9-γ1 mAb to Fas at 3, 6, 33, 66,132 nM mAb concentrations over the Fas receptor surface. The IgG1 concentrations are 2.12, 4.25, 17 and 68 nM. Black lines represent a best fit of the binding data to a bivalent analyte model.

FIG. 3B shows bivalent analyte fit of Fas/IgG4 interaction data. Colour lines are injections of IgG4 at different concentrations over Fas receptor surface. The IgG4 concentrations are 2.12, 4.25, 17 and 68 nM. Black lines represent a best fit of results of application of the BIAcore models to calculate the binding data to a bivalent analyte model and affinity constants. 1:1 binding model gave a nice fit using 3, 6 and 33 nM.

The generic structures of antibodies usually contain carbohydrate chains in the Fc region. In addition, two potential glycosylation sites in the variable region of the heavy chain (Fab) were suggested in F45D9 by in silico modelling (N-glycosylations) at . . . TNY . . . (N58) and . . . LNL . . . (N81). Both these asparagines are surface exposed residues in beta-sheets and as such could be actual glycosylation sites, also from a structural point of view. These carbohydrate structures could directly affect the actual binding of the antibodies to the Fas receptor. Carbohydrate structures at the specific suggested sites has not been identified but the presence of carbohydrates in the Fab region has been non-specifically established making it highly plausible to suggest the presence of carbohydrates at one or both of the suggested sites.

### EXAMPLE 5

### Epitope Mapping of F45D9-γ1 and F45D9-γ4 mAb

Epitope mapping of F45D9-γ1 and F45D9-γ4 mAb was done using peptide microarrays from JPT peptide technologies. The microarray is composed of several different peptide scans of TNR6_Human protein (FAS molecule, accession number P25445; Oehm A. et al. J. Biol. Chem. 267:10709, 1992), immobilized on a glass surface. The microarrays were pre-treated 2 h at room temperature with blocking buffer (Pierce, Superblock), followed by washings with TBS buffer, pH 8.0 and water (3 times each). Pre-treated arrays were scanned using Axon-4000B-Microarray Scanner for background control. Arrays were then incubated with F45D9-γ1 mAb (final concentration 50 ug/ml in assay buffer, Pierce, Superblock), followed by washings with TBS buffer pH 8.0 and further incubation with secondary antibody, anti-human-Cy5 (Jackson ImmunoResearch 209-175-082). Control incubation with secondary antibody only was performed in parallel. All microarrays were scanned using Axon-4000B-Microarray Scanner with appropriate wavelength settings. SPOT recognition software package ArrayPro was used for data analysis. Mean of signal intensities from 3 identical subarrays on each microarray image were used for data evaluation.

### Results

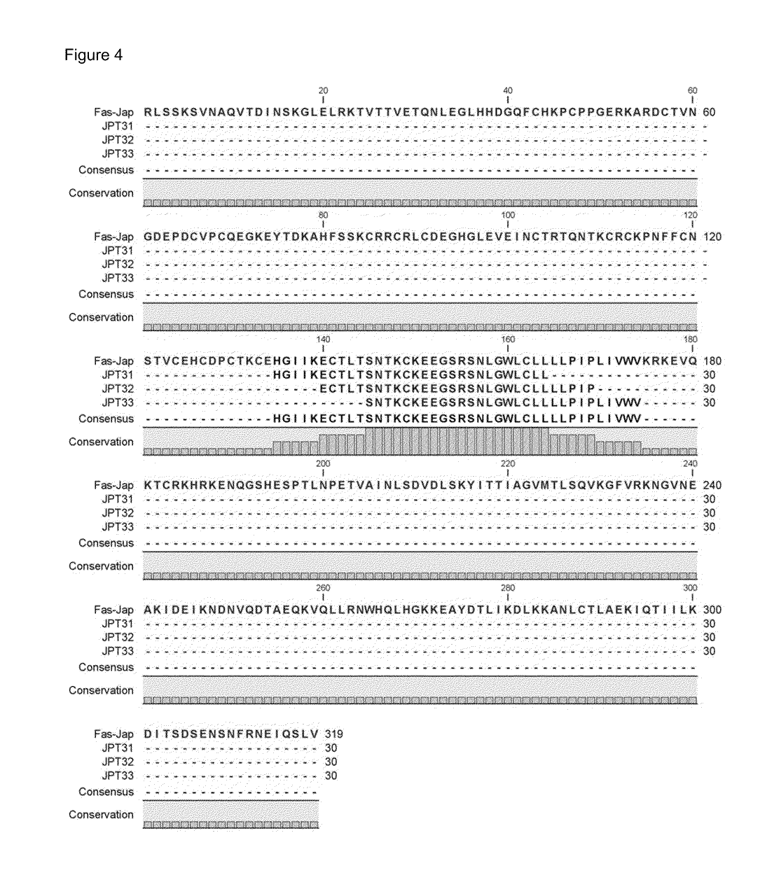

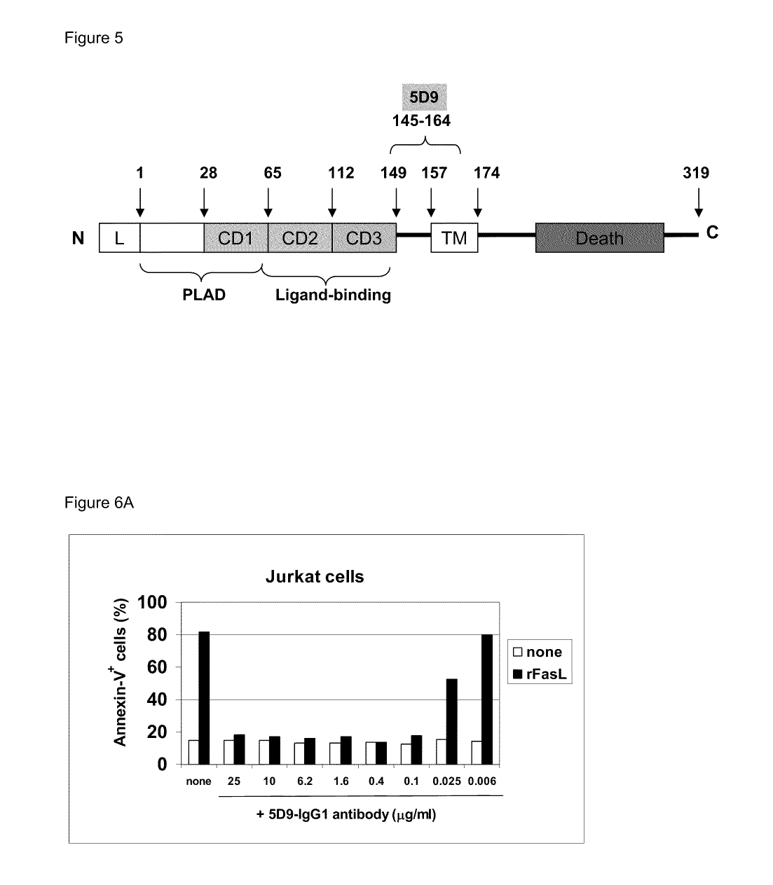

The epitope mapping analysis shows F45D9-γ1 mAb binding on TNR6_Human protein region aa169-aa191 (Oehm A. et al. J. Biol. Chem. 267:10709, 1992), which seems to be a linear epitope. The alignment with the Fas protein sequence (SEQ ID NO 11; Itoh, N. et al., Cell, 66:233-243, 1991) shows that F45D9-γ1 mAb binds to a common region of the 31, 32 and 33 binding peptides (SEQ ID NO:12, 13 and 14 respectively), corresponding to aa145-aa164, i.e. SNTKCKEEGSRSNLGWLCLL (SEQ ID NO.15) (FIGS. 4 and 5).

### EXAMPLE 6

### In Vitro Antagonistic Activity of F45D9-γ1, F45D9-γ4 mAb and Fab Fragments in rhFasL-Induced Apoptosis in Human T and B Cells

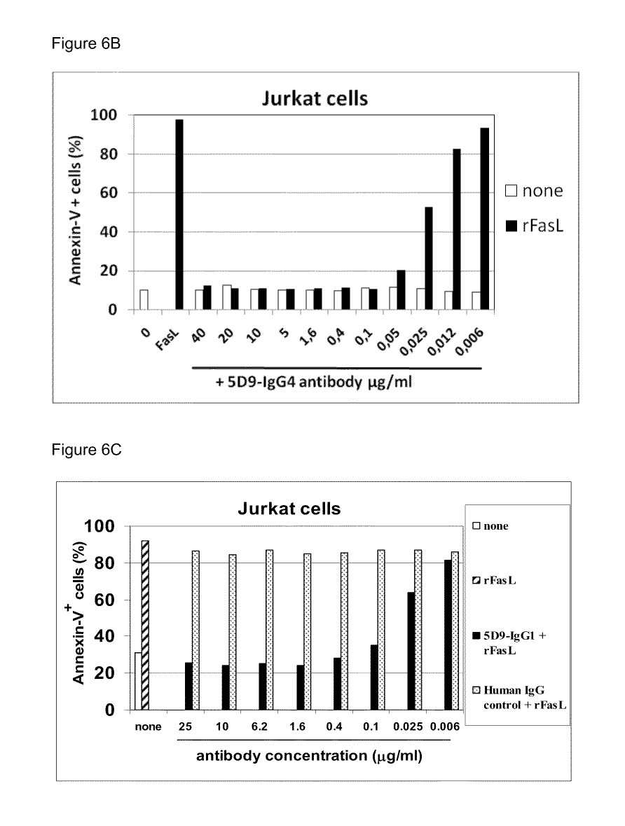

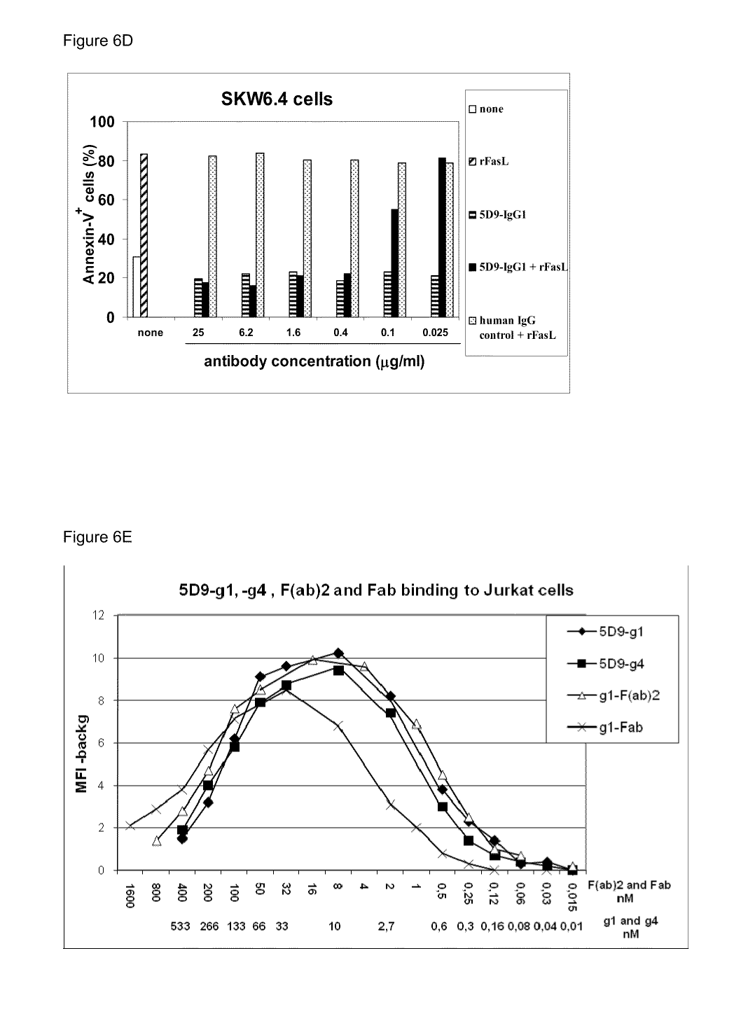

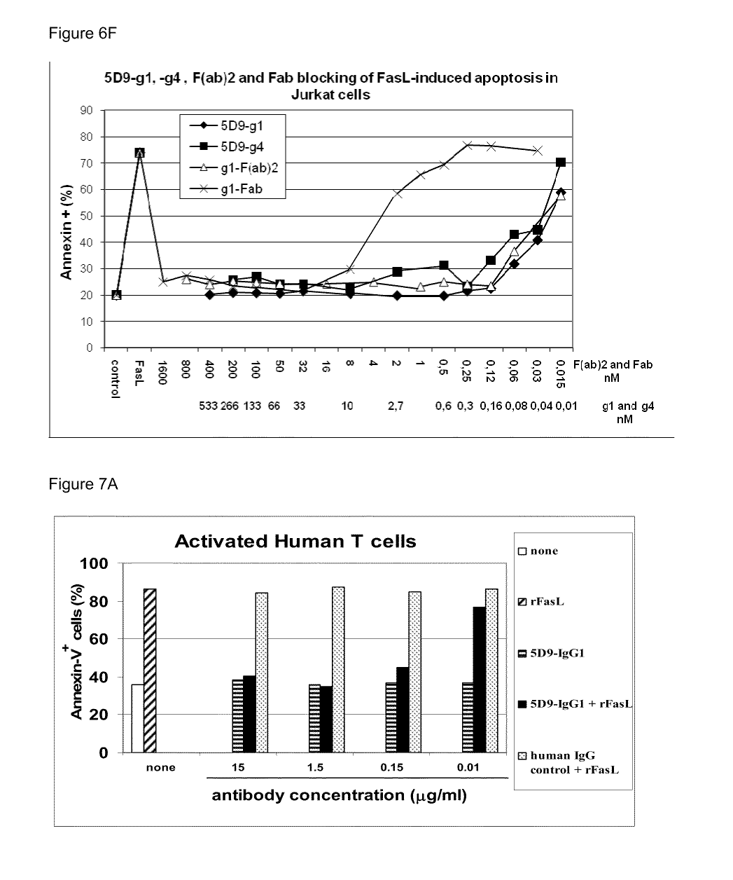

For assessing F45D9-γ1 and F45D9-γ4 mAb effect on apoptosis human T and B cell lines were induced to die in vitro with recombinant human FasL (rhFasL). Apoptosis was determined after Annexin-V and Propidium iode (PI) staining and flow cytometry analysis. Jurkat (FIG. 6A, FIG. 6B and FIG. 6C) or SKW6.4 cells (FIG. 6D) (0.2×10⁶ cells/well) were cultured overnight at 37° C. in 96-well U-bottom plates in 100 ul/well of RPMI 1640 medium supplemented with 2 mM glutamax, 100 UI/mL penicillin, 100 μg/mL streptomycin and 5% fetal bovine serum, alone or with 200 ng/ml of rhFasL (R & D Systems, Cat. 126-FL) plus 10 μg/ml anti-6× Histidine mAb (R & D Systems, Cat. MAB050). For assessing antibodies effect on apoptosis, cells were pre-incubated for 1 h at 37° C. with different concentrations of F45D9-γ1 or F45D9-γ4 mAb or human IgG control antibodies (Human IgG1, kappa). Apoptosis was determined by Annexin-V and PI staining by using the Annexin-V-FITC apoptosis detection kit (BD Biosciences, Cat. 556547) according manufacturer's instructions: cells were washed with PBS and incubated for 10 min, at RT in the dark, in 50 μl of binding buffer (10 mM HEPES/NaOH (pH 7.4), 140 mM NaCl, 2.5 mM CaCl₂) containing 2 ul of Annexin-V-FITC solution and 2 μl of propidium iodide solution. After adding 200 μL of binding buffer cells were analyzed immediately with a FACScan (FIGS. 6A, 6B, 6C and 6D).

Binding titration (FIG. 6E) and the effect on FasL-induced apoptosis (FIG. 6F) of F45D9-γ1, F45D9-γ4 mAbs, and F45D9-γ1 F(ab′)₂ and Fab fragments were studied in Jurkat cells, following the experimental procedure described in Example 2 and Example 6, respectively. FITC-conjugated rabbit F(ab′)₂ anti-human IgG (DakoCytomation Cat. F0056) was used as secondary antibody in FIG. 6E. Antibody concentrations are represented in nM to be able to compare whole molecule and fragments of antibodies.

### Results

It was demonstrated that F45D9-γ1 mAb or F45D9-γ4 mAb inhibits in vitro FasL-induced apoptosis in the human T cell line Jurkat in a dose dependent manner at a concentration in the range of 0.1 ug/ml to 25 ug/ml (IC50: 10-40 μM) and in the human B cell line SKW6.4 in the range of 0.4 ug/ml to 25 ug/ml (IC50: 200 pM). It was also shown that F45D9-γ1 mAb or F45D9-γ4 mAb alone does not induce apoptosis in these cell types.

In FIG. 6F it is shown that 5D9-γ1 Fab blocks FasL-induced apoptosis in Jurkat cells at a concentration 100 times more than for 5D9-γ1, 5D9-γ4 and 5D9-γ1 F(ab′)₂. This concentration correlates with saturation in binding with 5D9-γ1 Fab (FIG. 6D). However for 5D9-γ1, 5D9-γ4 and 5D9-γ1 F(ab′)₂ complete blocking in apoptosis is achieved when there is ≧12.5% of binding capacity (FIGS. 6E and 6F). These results suggest that 5D9 Fab mechanism of action might be by blocking FasL, but for 5D9-γ1, 5D9-γ4 and 5D9-γ1 F(ab′)₂, it might be some other mechanisms associated to activation of signals leading to apoptosis inhibition (ex. DISC formation related mechanism; Triggering of non-apoptotic Fas-mediated signaling leading to survival such as activation of Mitogen-activated protein kinase (MAPK), NFkB, c-Jun N-terminal Kinase (JNK), AKT)

### EXAMPLE 7

### In Vitro Antagonistic Activity of F45D9-γ1 mAb and F45D9-γ4 mAb in Activation Induced Cell Death (AICD) in Human T Cells

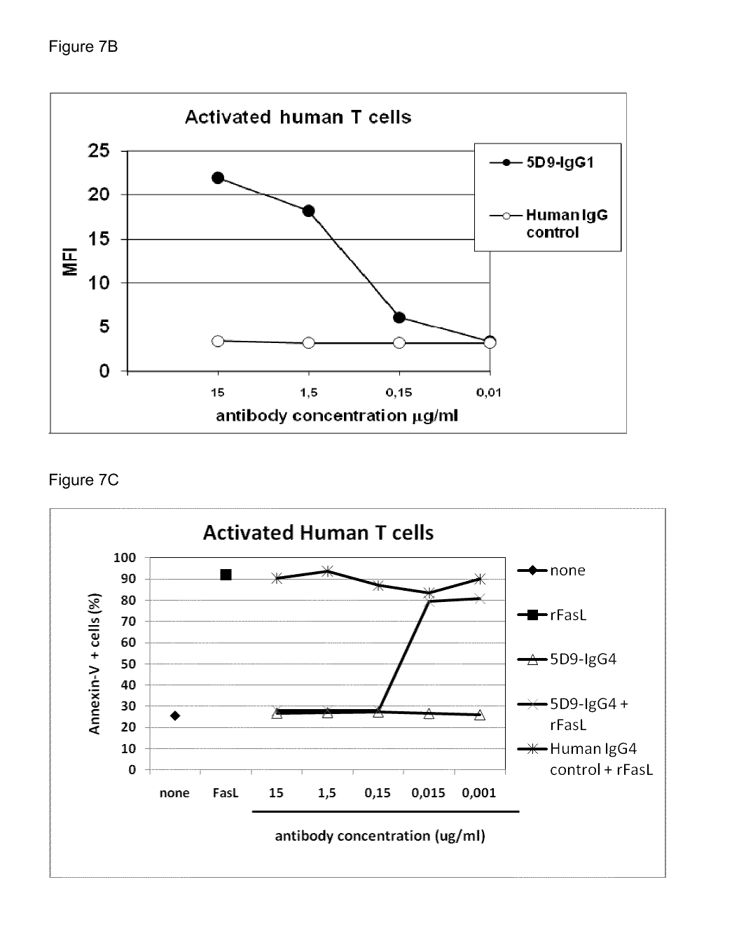

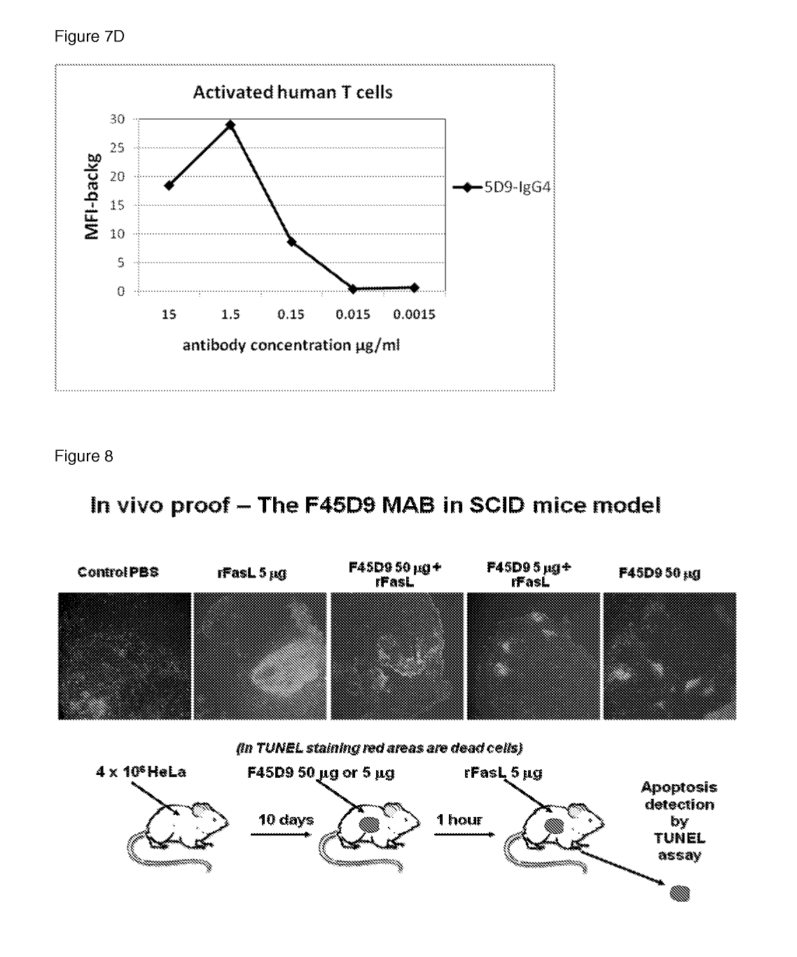

FIG. 7A and FIG. 7C shows results of the experiments demonstrating blocking of Activation Induced Cell Death (AICD) in human T cells by F45D9-γ1 and F45D9-γ4, respectively.

Preparation of primary T cells: Human PBMC (Peripheral Blood mononuclear cells) were isolated from venous blood samples from healthy volunteer donors by Lymphoprep (Fresenius Kabi Norge AS for Axis-Shield PoC AS, Oslo, Norway) density gradient centrifugation. Then T cells were obtained by negative selection using a Pant T cell isolation kit II (human, MACS, Miltenyi Biotech Inc., Cat. 130-091-156) according to manufacturer's instruction. This isolation procedure routinely yielded a population of T cells that was 90% CD3 positive as assessed by flow cytometry.

Activation of T cells was done as described by Schmitz, I, et al (J. Immunol., 171:2930-2936, 2003). Briefly, resting T cells (day 0) were cultured in T25 flask at 2×10⁶ cells/ml in RPMI 1640 medium supplemented with 2 mM glutamax, 100 UI/mL penicillin, 100 μg/mL streptomycin and 10% fetal bovine serum containing 1 ug/ml PHA for 16 h (day 1). Day 1 T cells (>95% CD3 positive) were then washed three times with PBS and cultured for an additional 5 days in the presence of 25 U/ml IL-2 (day 6). On day 6, dead cells were removed by density centrifugation. Cells were washed with medium and resuspended in fresh medium. Activated T cells were then used to assess F45D9-γ1 or F45D9-γ4 mAb effect on apoptosis induced in vitro with recombinant human FasL (rhFasL) as described in Example 6. Activated human T cells were also stained with F45D9-γ1 (FIG. 7B) or F45D9-γ4 (FIG. 7D) mAb followed by FITC-conjugated rabbit F (ab')₂ anti-Human kappa light chain or anti-IgG Fc antibodies (Dako Cytomation Cat. F0434 or F0056) as described in Example 2. The reactivity of F45D9 to Fas antigen was quantified by flow cytometry and represented in MFI. Forward and side scatter gates were set to exclude dead cells. Data from a representative of two experiments with different donors are shown.

### Results

It was demonstrated that F45D9-γ1 or F45D9-γ4 mAb inhibits in vitro FasL-induced apoptosis in activated human T cells in a dose dependent manner at a concentration in the range of 0.1 ug/ml to 15 ug/ml (IC50: 20 pM). It was also shown that F45D9-γ1 or F45D9-γ4 mAb alone does not induce apoptosis in activated human T cells.

### EXAMPLE 8

### In Vivo Antagonistic Activity of F45D9-γ1 mAb In FasL-Induced Cell Death (SCID Mice Model)

Tumor inoculation in SCID mice and antibody treatment: Female CB.17 SCID/SCID mice aged 4-5 weeks (Harlan; Correzzana, Milan, Italy) were kept under specific pathogen-free conditions. Each mouse was injected subcutaneously in the right flank with 4×10⁶ HeLa cells (human cervical carcinoma cell line; purchased from American Type Culture Collection (ATCC)) that had been resuspended in 0.2 ml of RPMI-1640. After 10 days, a selection of mice with a tumor diameter of about 0.5 cm was made. F45D9-γ1 mAb (50 or 5 ug) resuspended in PBS and in 100 ul volume was injected directly into the tumor, followed 1 hour later by injection in the same place of 100 ul/mouse of a mix of 5 ug/mouse of rhFasL (R & D System, Cat. 126-FL) and 50 ug/mouse of monocolonal anti-6× Histidine (R & D System, Cat. MAB050) diluted in PBS and that was preincubated for 1 hour at 37° C. Each animal from the control group was only injected with 100 ul/mouse PBS into the tumor. After 24 hours the animals were sacrificed and tissue sections were processed for apoptosis detection by TUNEL assay. Three mice were used for each treatment group.

TUNEL assay: Frozen sections were fixed with 4% paraformaldehyde and permeabilized with 0.1% Triton X-100, 0.1% sodium citrate. The TUNEL reaction was performed according to manufacture instructions (In situ Cell Death detection kit, TMR red (Roche, Cat. 2 156792). Slides were rinsed, counter stained, and analyzed under a light microscope. Positive control sections included in each assay were DNase treated.

### Results

As demonstrated by the images shown in FIG. 8, there is a decreased cell staining in the presence of F45D9-γ1 mAb, indicating reduced levels of FasL-induced cell death in F45D9 treated tumors versus untreated tumors (only injected with rhFasL). These results confirmed the in vitro data that provides indication that the F45D9-γ1 mAb can block apoptosis via FasL. The treatment with F45D9-γ1 mAb alone did not increase cell death over the background level, confirming the in vitro data of no apoptosis induction by F45D9-γ1 mAb alone.

### EXAMPLE 9

### Reactivity of F45D9-γ1 and F45D9-γ4 mAb with Fas Molecues of Non-Human Primates Marmoset and Chimpanzee

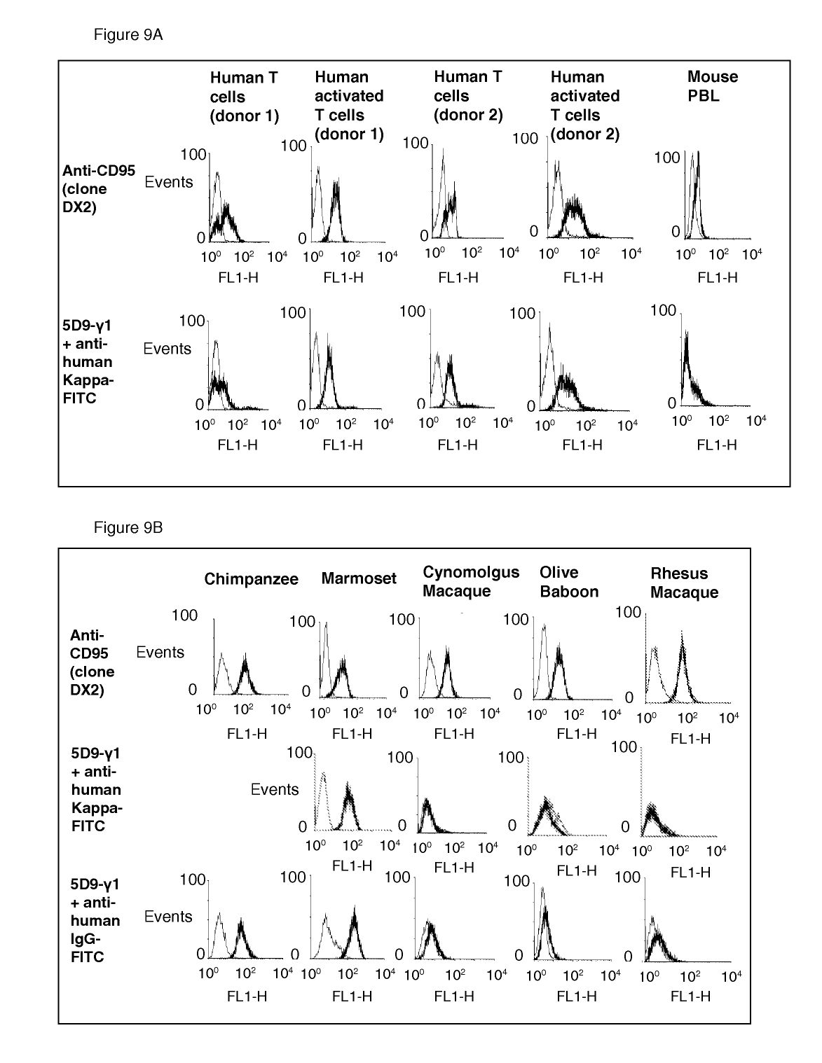

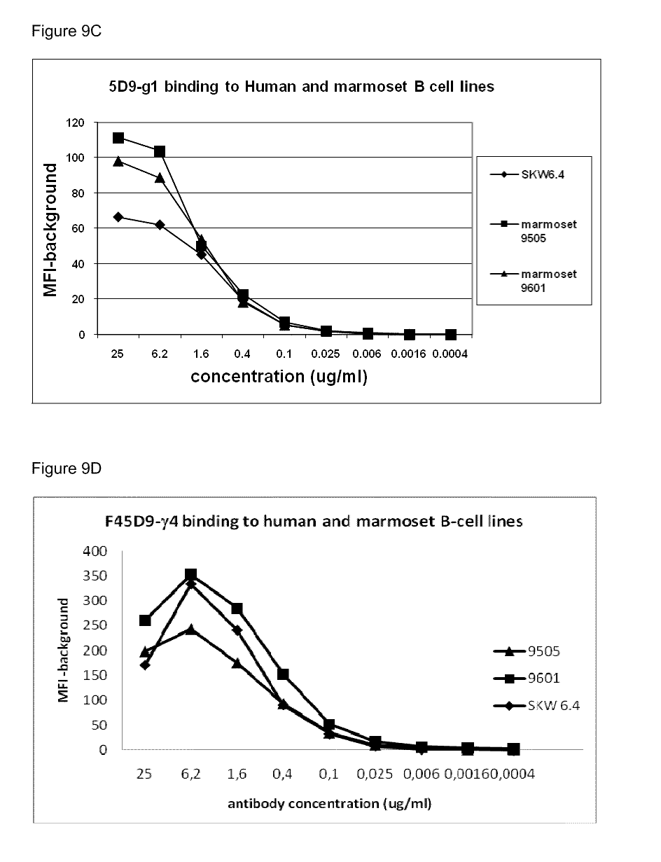

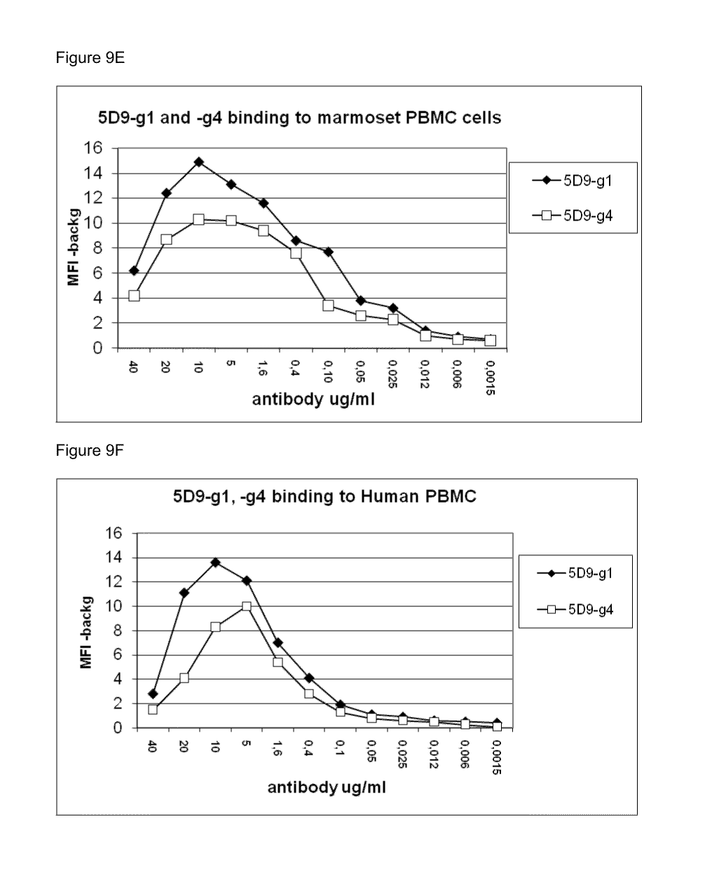

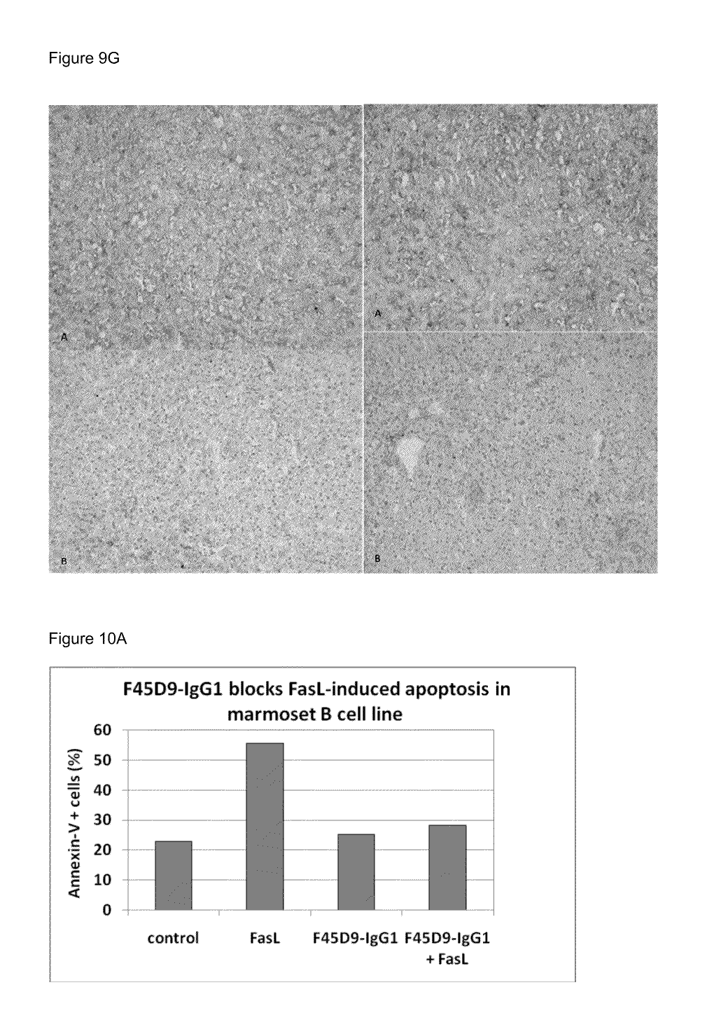

Reactivity of F45D9-γ1 or F45D9-γ4 mAb with Fas molecules of various species was initially screened using primary blood cells or lymphocytes cell lines. The binding of F45D9-γ1 or F45D9-γ4 mAb was explored by immunofluoresce staining and flow cytometry analysis, in human PBMC, purified human resting T cells, activated human T cells, peripheral blood lymphocytes (PBL) derived from BALE/c mice, rat, dog and pig, and in B cell lines or PBL derived from common marmoset, cynomolgus macaque, olive baboon, rhesus macaque and chimpanzee. Resting and activated human T cells were prepared as described in Example 7. B cells lines from monkeys were obtained from Biomedical Primate Research Center (Rijswijk, The Netherlands), and were cultured in RPMI 1640 medium supplemented with 2 mM glutamax, 100 UI/mL penicillin, 100 μg/mL streptomycin and 10% fetal bovine serum, at 37° C. and 5% CO₂. After washing in PBS cells (0.2−0.4×10⁶ cells/well) were incubated in staining buffer (PBS/1% FBS) containing F45D9-γ1 or human IgG control antibodies (10-20 ug/ml) in a 96-well U-bottom plate for 30 min on ice, in a 100 ul/well volume. Cells were then washed once with staining buffer, and further incubated for 30 min on ice with 1:30 dilution of FITC-conjugated rabbit F(ab′)₂ anti-human kappa (DakoCytomation Cat. F0434) or FITC-conjugated rabbit F(ab′)₂ anti-human IgG (DakoCytomation Cat. F0056). FITC-conjugated mouse anti-human CD95 mAb (Clone DX2, BD Biosciences, cat. 555673) was used as positive control for Fas staining in human and monkey cells, while 20 ug/ml of hamster anti-mouse Fas (Jo2 mAb, BD Pharmingen, Cat. 15400D) followed by 20 ug/ml FITC-conjugated anti-Armenian and Syrian hamster IgG (BD Pharmingen, Cat. 444011) were used for positive Fas staining in mouse PBL. The reactivity of the antibodies to Fas antigen was quantified by flow cytometry and histograms are shown in FIG. 9A and FIG. 9B. The bold solid line indicates staining with anti-Fas antibodies and the light solid line represents staining by isotype control antibodies. Forward and side scatter gates were set to exclude dead cells. Data from a representative of two experiments are shown.

For comparison of F45D9-γ1 and F45D9-γ4 binding affinity between human and marmoset cells, the reactivity of different concentrations of F45D9-γ1 and -γ4 mAbs to Fas on human B cell line (SKW6.4) and marmoset B cell lines (established from two animals, 9505 and 9601) was quantified by flow cytometry using as secondary antibodies FITC-conjugated rabbit F(ab′)₂ anti-human IgG (DakoCytomation Cat. F0056), as described before. Results in MFI after subtraction of background staining with control human γ1 and γ4 are shown in FIGS. 9C and 9D. Forward and side scatter gates were set to exclude dead cells. Data from a representative of two experiments are shown.

For comparison of F45D9-γ1 and -γ4 binding affinity between human and marmoset the binding titration (range from 40-0.0015 ug/ml) was also done using PBMC isolated from one marmoset animal and from human healthy donor. The staining procedure was as described before, using as secondary antibodies FITC-conjugated rabbit F(ab′)₂ anti-human IgG (DakoCytomation Cat. F0056). Results are shown in FIG. 9E (marmoset PBMC) and 9F (human PBMC) as MFI, after subtraction of background staining with control human γ1 and γ4.

Binding of F45D9-γ4 mAb mAb to human and marmoset tissues was assessed in an immunohistochemical cross-reactivity study. Frozen human and marmoset tissues were cryo sectioned at 8 μm, and after fixation in cold acetone the sections were stained with FIT-labelled F45D9-γ4 mAb or isotype control, respectively and developed using secondary mouse anti-FITC and anti-mouse-HRP antibodies.

### Results

We have demonstrated that F45D9 antibodies, both γ1 and γ4 isotypes bind to Fas on human lymphocytes, as wells as lymphocytes from non-human primates chimpanzee and common marmosets. However, none or very low percentages of lymphocytes from mouse (FIG. 9A), rat, dog, pig or other non-human primate species (FIG. 9B and Table 2) reacted with F45D9 mAb. The results shown in FIGS. 9C and D indicate a similar binding affinity of F45D9 between human and marmoset cells. In addition FIGS. 9E and 9F show similar binding titration curves for F45D-γ1 and γ4 antibodies on marmoset and human primary lymphocytes.

The immunohistochemical cross-reactivity study in human and marmoset tissues showed that F45D9-γ4 mAb reacted with many of the investigated tissues. All lymphoid organs showed marked reactivity, and other positive tissues were liver, adrenal, epithelia in skin, esophagus and uterine cervix, among others. The reaction pattern was similar when comparing human and Marmoset tissues. F45D9-γ4 mAb reactivity with human and common marmoset liver tissue is illustrated in FIG. 9G.

### EXAMPLE 10

### In Vitro Antagonistic Activity of F45D9-γ1 and F45D9-γ4 mAb in rhFasL-Induced Apoptosis in B Cells and Activated Lymphocytes from Nonhuman Primate Common Marmoset

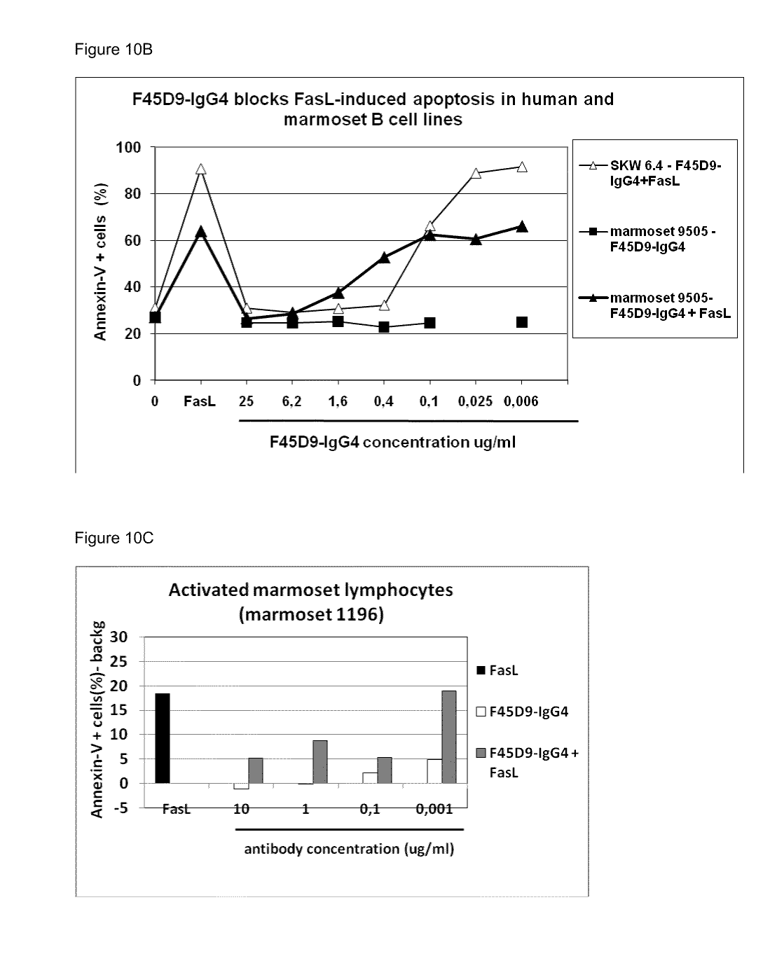

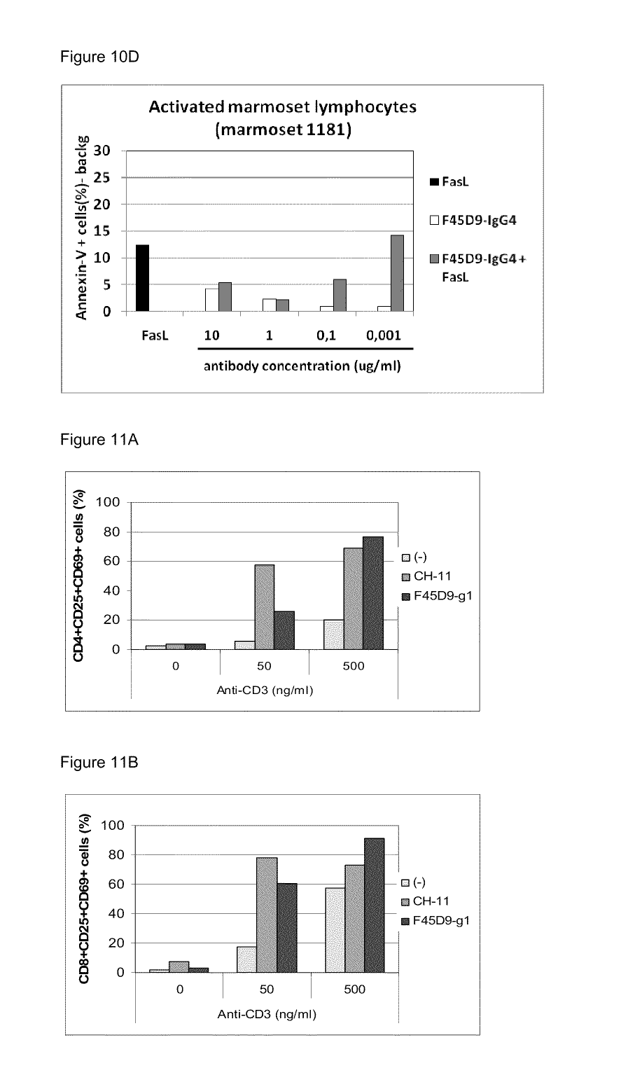

The antagonistic activity of F45D9-γ1 or F45D9-γ4 mAb on rhFasL-induced apoptosis was assessed in vitro in immortalized B cells isolated from common marmoset, as described in Example 6. B cell line derived from two common marmoset monkey (marmoset 9601 in FIG. 10A; marmoset 9505 in FIG. 10B)) were obtained from Biomedical Primate Research Center (Rijswijk, The Netherlands), and were cultured, before using in the assay, in RPMI 1640 medium supplemented with 2 mM glutamax, 100 UI/mL penicillin, 100 μg/mL streptomycin and 10% fetal bovine serum, at 37° C. and 5% CO₂. The human B cell line SKW6.4 was also used to compare the antagonistic activity of F45D9-γ4 mAb in both human and marmoset B cell lines (FIG. 10B). For assessing antibodies effect on apoptosis, cells were pre-incubated for 1 h at 37° C. with F45D9-γ1 mAb (10 ug/ml) or different concentrations of F45D9-γ4 mAb (25-0.006 ug/ml) in 100 ul/well of medium, or with medium alone as control. Apoptosis was determined after Annexin-V and Propidium iode (PI) staining and flow cytometry analysis as described in Example 6. Data from a representative of two experiments are shown in FIGS. 10A and 10B.

The antagonistic activity of F45D9-γ4 mAb on apoptosis induced in vitro with recombinant human FasL (rhFasL) was also studied in activated lymphocytes isolated from common marmoset, as described in Example 6. PBMC were isolated from two common marmoset animals (marmoset 1196 and marmoset 1181) and activated as described in Example 7.

### Results

It was demonstrated that F45D9-γ1 or F45D9-γ4 mAb mAb inhibits in vitro FasL-induced apoptosis in marmoset B cells in a dose dependent manner at a concentration in the range of 6 ug/ml to 25 ug/ml (FIGS. 10A and 10B). It was also shown that F45D9-γ1 or F45D9-γ4 mAb alone does not induce apoptosis in this cell type.

In FIG. 10C (marmoset 1196) and FIG. 10D (marmoset 1181) it is shown that F45D9-γ4 mAb inhibits in vitro FasL-induced apoptosis of activated marmoset lymphocytes in a dose dependent manner at a concentration in the range of 0.1 ug/ml to 10 ug/ml. It was also shown that F45D9-γ4 mAb alone does not induce apoptosis in activated marmoset lymphocytes. The results demonstrate similar antagonistic activity of F45D9-γ4 mAb in human and marmoset lymphocytes.

### EXAMPLE 11

### F45D9-γ1-Mediated Co-Stimulatory Signal in Activation and Proliferation of Human T Cells

Since Fas has been described as a co-stimulatory factor during T cell activation, we investigated the potential of F45D9-γ1 mAb to co-stimulate T cell activation and proliferation.

Human T cells were purified as described in Example 7 and were cultured for 3 days with solid-phase-bound anti-CD3 monoclonal antibody at a suboptimal concentration with or without F45D9-γ1 or CH-11 mAb, which is a well-characterized mouse anti-Fas mAbs that served as control. T cell activation was assessed measuring the expression of activation markers, namely CD25 (IL-2 Receptor) and CD69 on CD4+ and CD8+ T cells. T cell proliferation was assessed measuring ³H-thymidine incorporation and FACS analysis of CFSE labelled cells.

### 1. Effect of F45D9-γ1 mAb on Activation of Human T Cells

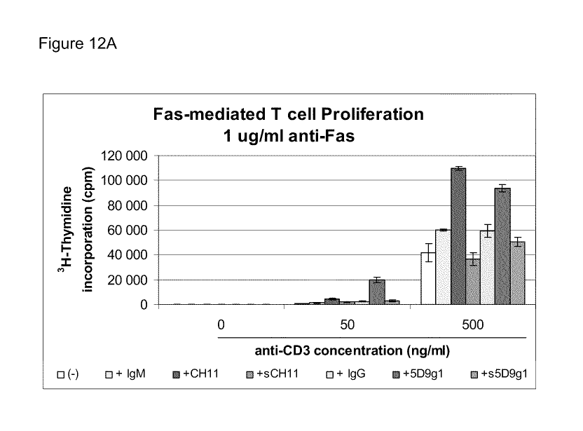

96-well flat bottom plate (Corning Incorporated, Costar 3590) was coated overnight at 4° C. with mouse anti-human CD3 mAb (Clone HIT3a, BD Pharmingen, Cat. 555336) at a concentration of 500 or 50 ng/ml in 100 μl PBS with or without F45D9-γ1 or mouse anti-Fas CH-11 (MBL, Cat. SY-001) antibodies at concentration 1 ug/ml. The wells were washed once with medium before seeding 1,5×10⁵ purified T cells resuspended in 200 μl medium (RPMI 1640 medium supplemented with 2 mM glutamax, 100 UI/mL penicillin, 100 μg/mL streptomycin and 10% fetal bovine serum). After 3 days of culture, the cells were harvested, washed with PBS and stained with APC-conjugated anti-CD4 or -CD8, PE-Cy5-conjugated anti-CD69 and PE-conjugated anti-25 (BD Pharmingen): cells were incubated with the antibodies for 30 minutes at +4° C. in the dark. After washing in PBS, cells were fixed in 1% paraformaldehyde in PBS. FACS analyses were done by using a FACScalibur flow cytometer and CellQuest Pro (Beckton-Dickinson) software. Routinely, 10,000 cells were collected for analyses and gated using the side/forward scatter; additionally CD4 or CD8 positive cells were gated before the analysis of CD25 and CD69 expression. Percentage of CD25 and CD69 double positive cells on CD4+ and CD8+ cells were determined as shown in FIGS. 11A and 11B, respectively. Data are representative of three independent experiments.

### Results

Sub-optimal concentrations of anti-CD3 (50 and 500 ng/ml) were chosen not to give by itself a signal allowing a fully activation and a proliferation of the T cells. Without any activation, less than 10% of both CD4+ and CD8+ T cells expressed CD25 and CD69. Activation of T cells with solid bound anti-CD3 and F45D9-γ1 mAbs led to a dramatic increase in both activation markers in CD4+ and CD8+ cells, as compared with cells cultured with coated anti-CD3 alone, even at low concentration of 50 ng/ml anti-CD3 mAbs. Interestingly, CD8+ T cells up-regulated the membrane expression of CD25 and CD69 more than CD4+ T cells. Both F45D9-γ1 and CH-11 mAbs acted similarly on activation marker expression. Thus, F45D9-γ1 mAb might mediate co-stimulatory signal in the activation of human T cells.

### 2. Effect of F45D9-γ1 mAb on Proliferation of Human T Cells

96-well flat bottom plate (Corning Incorporated, Costar 3590) was coated overnight at 4° C. with mouse anti-human CD3 mAb (Clone HIT3a, BD Pharmingen, Cat. 555336) at a concentration of 500 or 50 ng/ml in 100 μl PBS with or without F45D9-γ1 or mouse anti-Fas CH-11 (MBL, Cat. SY-001) antibodies at concentration 1 ug/ml. Human IgM or Human IgG1kappa (Sigma, Cat. 18260 and I5154, respectively) were used as negative controls. The wells were washed once with medium before seeding 1.5×10⁵ purified T cells resuspended in 200 μl medium. In some wells coated only with anti-CD3 mAbs the anti-Fas antibodies were added together with the cells to assess the antibody effect of their soluble form. The proliferation was assessed after 3 days of culture measuring ³H-thymidine incorporation: cells were pulse for the last 18 hours with 20 μl/well (1 μCi/well) of 50 μCi/ml [methyl-³H]-thymidine (Sigma). The cells were harvested on day 3 onto a glass fiber filters and sealed with Liquid Scintillation cocktail (Betaplate Scint.). Then the ³H-thymidine uptake was determined using a Liquid Scintillation counter (1450 Microbeta), and results are expressed in cpm. The experiments were done in triplicate.

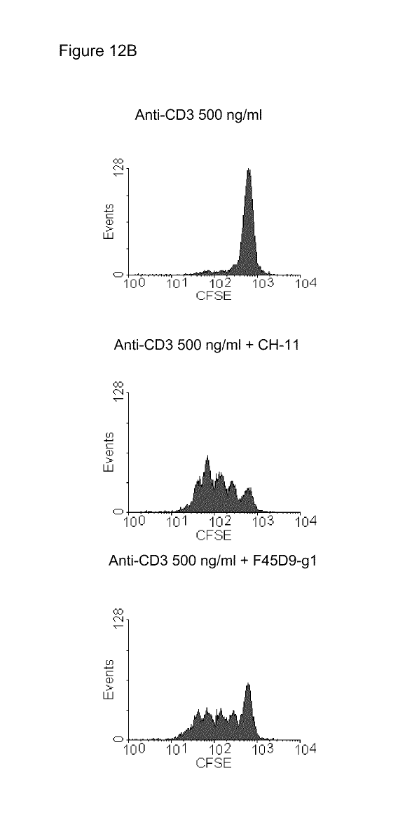

In parallel, purified T cells were stained with CFSE. The cells were washed once in phosphate buffered saline (PBS) and diluted in 10 ml pre-warmed PBS containing 0.1 μM CFSE (Molecular Probes). Following 15 minute-incubation at 37° C., the T cell suspension was washed with pre-warmed medium and incubated for an additional 30 minutes at 37° C. CFSE-stained T cells were counted and resuspended in medium 10% FCS to be added (1.5×10⁵/well) to the plates and activated in the same conditions as mentioned above. CFSE-labeled cells were harvested after 3 days of culture and analyzed by FACS (Becton-Dickinson) to assess proliferation. Data shown in FIG. 12B are representative of two different experiments.

### Results

Both F45D9-γ1 and CH-11 mAbs increased the proliferation of cells culture with sub-optimal concentrations of anti-CD3 (500 ng/ml), although it was slightly higher with CH-11. Interestingly, with less anti-CD3 (50 ng/ml), only F45D9-γ1 mAb gave proliferation signal to T cells, however the response varied between donors. Cells incubated with ZB4, another anti-Fas blocking antibody, showed only a slight increase in the proliferative response of cells cultured with suboptimal concentration of anti-CD3. With APO-1-1 as anti-Fas mAbs, the proliferation observed was not higher than with isotype control. When F45D9-γ1 or CH-11 mAbs were added in soluble form to the coated anti-CD3 cultures, no increase in the proliferative response was observed, as previously reported for CH-11 [33]. Thus, F45D9-γ1 mAb may be used to mediate co-stimulatory signal in the proliferation of human T cells.

### EXAMPLE 12

### Effect of F45D9-γ1 and F45D9-γ4 mAb in Inducing Antibody Dependent Cell Mediated Cytotoxicity (ADCC)

The effect of F45D9-γ1 and F45D9-γ4 mAb in inducing ADCC was evaluated by quantifying the release of ⁵¹Cr from lysed SKW6.4 cells, after incubation with PBMC and antibody.

A sufficient volume of SKW6.4 cells was centrifuged for 5-8 minutes at approximately 200 g before being washed in RPMI1640 medium. The supernatant was discarded and the cells re-suspended by tapping the tube without the addition of extra medium and allowed to equilibrate for up to 15 min at 37±2° C. in 5% CO₂ in air. ⁵¹Cr was added to the cells to give 1.11 MBq of activity per 10⁶ cells and incubated at 37° C. for 1.5 hr. Cells were gently shaken approximately every 15 min, and then washed three times in medium to remove excess ⁵¹Cr. The washed cells were re-suspended in medium at 2×10⁵ cells/ml.

To obtain the PBMCs, human blood was drawn into EDTA tubes and diluted 1 in 2 in PBS, and PBMCs was then isolated by ficoll density gradient. The separated cells were washed twice in PBS and re-suspended at 1×10⁷ cells/ml in media. The assay was performed in triplicate wells in U-bottomed 96-well microtiter plates. Fifty microliters of target cell suspension (1×10⁴ cells) were added to each well in the presence of varying concentrations (1; 0.5; 0.25; 0.125; 0.06; 0.03; 0.015 and 0.008 μg/ml) of 5D9-≢1 or 5D9-γ4 antibodies, respectively. Target cells were incubated with positive control antibodies (Rituximab) or a non-binding IgG1 or IgG4 negative control antibody (20 ug/ml). Cells were then incubated for 1 h at 37±2° C. in a humidified atmosphere of 5% CO₂ in air prior to the addition of PBMC at a 50:1 ratio (50 μl of 1×10⁷ cells/ml). The cultures were then incubated for a further 4 hrs at 37±2° C. in a humidified atmosphere of 5% CO₂ in air. At the end of the 4 hr incubation period, assay plates were centrifuged for 5 min at 400 g. A volume of 100 μl of the supernatant was gently removed into 5 ml vials for gamma counting. A non-binding antibody was used as a negative control and Rituximab as positive control antibody. Additional control wells using PBMC incubated with target cells, but no antibody were prepared to determine the background level of non-antibody dependent cell lysis and wells containing target cells and antibody but no PBMC were prepared as control for any lytic effects of the antibody. SKW6.4 cells were incubated without PBMC to derive ⁵¹Cr spontaneous release data and with 1% triton X-100 to establish maximum release.

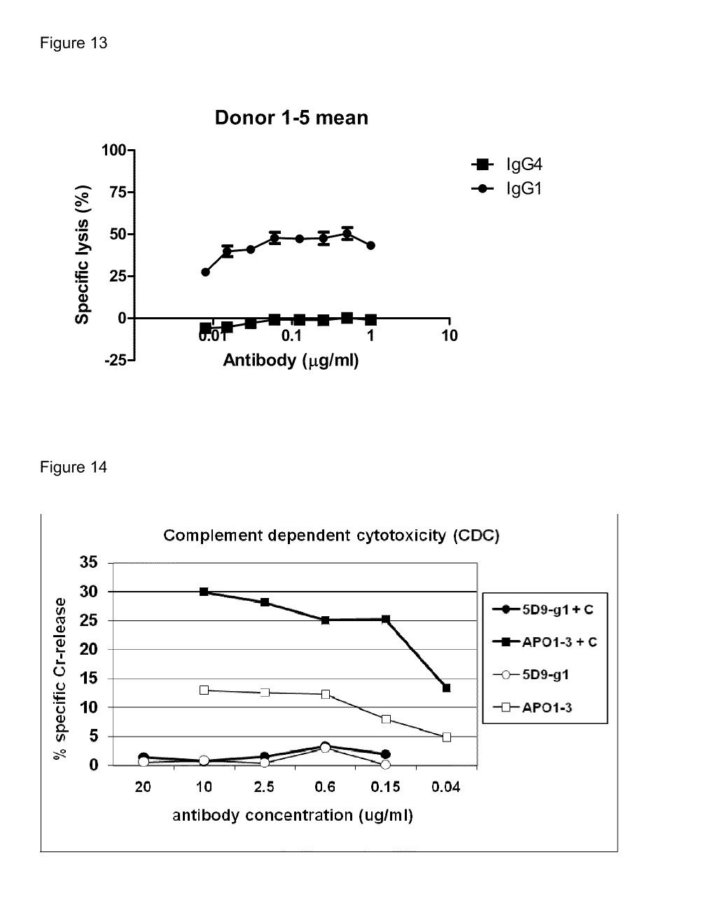

Results are expressed as a percent of specific lysis (exp release-background release/maximum release-background release)×100. Results from 5 donors expressed as mean of % of specific lysis are shown in FIG. 13.

### Results

Using 6 donors a very poor specific lysis induction was demonstrated by F45D9-γ4 mAb. However, a dose related increase in lysis was observed in the presence of F45D9-γ1 mAb in all six donors. It was found that the lysis reached its peak at 0.06 uM of F45D9-γ1 mAb. Results showed in FIG. 13 represent the mean of specific lysis from 5 donors (one of the donor showed poor response in the presence of F45D9-γ1 mAb, and was therefore removed from the plot of mean response).

We have shown that F45D9-γ1 mAb causes a dose dependent ADCC effect using SKW6.4 cells as target cells. F45D9-γ4 mAb caused very little ADCC of the SKW6.4 cells.

### EXAMPLE 13

### Effect of F45D9-γ1 mAb in Inducing Complement Dependent Cytotoxicity (CDC)

The effect of F45D9-γ1 mAb in inducing CDC was evaluated by an assay using release of ⁵¹Cr from Jurkat target cells. Jurkat cell pellet were labeled with 100 μCi of ⁵¹Cr (Na₂⁵¹CrO₄ stock 10 mCi/ml, Amersham) per 1×10⁶ cells for 1 h at 37° C. After washing the cells three times with complete culture medium (RPMI 1640 medium supplemented with 2 mM glutamax, 100 UI/mL penicillin, 100 μg/mL streptomycin and 5% fetal bovine serum), 1×10⁴ cells in 100 μl of medium were distributed in 96-well U-bottom plates (Corning Incorporated, Costar) that were previously filled with 50 ul/well of various concentrations of 5D9-γ1 or APO-1-3 (mouse IgG3 anti-human Fas, Alexis, Cat. ALX-805-020-C100) antibodies, diluted in medium. APO-1-3 mAb was used as a positive control of the assay. Then 50 μl/well of rabbit sera complement (Sigma Cat. S-7764) to a final dilution of 1/25 or culture medium were added. Plates were incubated for 4 h at 37° C. in 5% CO₂. Radioactivity was determined in a 50 μl aliquot of each supernatant obtained after a centrifugation at 200×g for 5 minutes. Wells of labelled Jurkat cells cultured in medium alone served as spontaneous release. Maximal ⁵¹Cr-release was determined by lysing the cells with 100 μl of 1% Tween-20 (Sigma-Aldrich, Cat. P1379). All tests were done in triplicates. The percentage of specific lysis was calculated as follows:

$$\frac{( experimental {}^{51} Cr \text{-} release ) - ( spontaneous {}^{51} Cr \text{-} release )}{( maximal {}^{51} Cr \text{-} release ) - ( spontaneous {}^{51} Cr \text{-} release )} \times 100$$

### Results

Results from FIG. 14 demonstrated that F45D9-γ1 mAb does not induce complement dependent cytotoxicity at a concentration in the range of 0.15 ug/ml to 20 ug/ml (shown here until 10 ug/ml). As described before (Dhein, J., et al. J. Immunol., 149:3166-3173, 1992) APO-1-3 mAb, used as positive control, induced CDC, which it was two times higher than cytotoxicity induced by the antibodies in the absence of complement rabbit sera, at a concentration in the range of 0.04 ug/ml to 10 ug/ml.

### EXAMPLE 14

### In Vitro Toxicity Test of F45D9-γ1 mAb, in Primary Human Hepatocytes

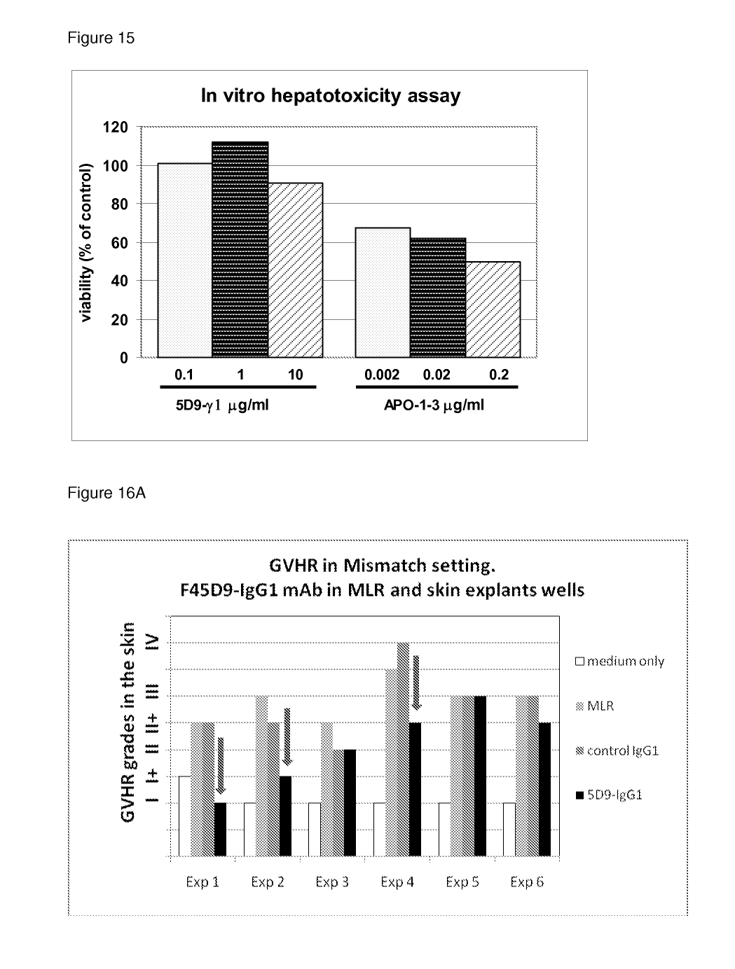

The hepatotoxicity effect of F45D9-γ1 mAb was tested in vitro using in primary human hepatocytes and XTT assay to determine cell viability. Human primary hepatocytes were obtained from Cambrex (Cat. No. CC-2591). Hepatocytes were dispensed into a type I collagen-coated 96-well plate (Beckton Dickinson, Cat. 354407) at 6×10⁴ cells/well in 150 ul/well of human epidermal growth factor (hEGF)-reconstituted hepatocyte culture medium (HCM) (Bullekit, Cambrex, Cat. No. CC-3198). After 3 h of incubation at 37° C., the plate was washed with warm medium to remove unattached cells. The hepatocytes were incubated with 150 ul/well of EGF-reconstituted HCM containing various concentrations of 5D9-γ1 or mouse anti-Fas APO-1-3 mAbs for 7 h at 37° C. Mouse anti-Fas antibody APO-1-3 induces hepatotoxicity and was used as positive control (Galle, P. R., et al., J.E.M 182:1223-1230, 1995). The viability of hepatocytes was determined by XTT assay, adding 75 ul/well of XTT reagent, incubating the plate overnight at 37° C., and then reading absorbance at A492 nm-A690 nm. Means and SD of triplicates were calculated. Results shown in FIG. 15 are expressed as percent of control in which cells were cultured in medium without antibodies.

### Results

It was demonstrated that F45D9-γ1 mAb does not induce hepatotoxicity at a concentration in the range of 0.1 ug/ml to 10 ug/ml. As described before (Galle, P. R., et al., J.E.M 182:1223-1230, 1995) APO-1-3 mAb, used as positive control, was hepatotoxic at a concentration in the range of 0.002 ug/ml to 0.2 ug/ml.

### EXAMPLE 15

### Pilot Toxicity Study in Marmosets with F45D9 mAb

The results from previous studies indicate that the marmoset is suitable for testing of safety and biological activity of F45D9-γ4. Therefore tolerability studies were done in common marmosets (Callithrix jacchus; Biomedical Primate Research Center, Rijswijk, The Netherlands. In the first part of the study, three marmoset monkeys were sequentially injected (i.v.) in a dose escalating study with 0.05, 0.5, and 5 mg/kg of 5D9-γ1, respectively, over approximately a 2 month period (at day 0, 35, and 70). One marmoset monkey was injected in parallel with IVIG control IgG (Gammagard S/D, Baxter, Belgium) at respective dose. The bleeding schedule was identical for each cycle of dosing. Blood samples were taken 3 days prior to injection, 1 h post infusion and on day 1 and 3 after injection. Clinical chemistry and haematology was determined and serum was stored for pharmacokinetics measurements. On day 77 or 78 the animals were euthanized for complete necropsy and pathological examination. PBMC were isolated and stored.

In a second part of the study three animals were injected (i.v.) with a single dose of 5 mg/kg of F45D9 of isotype IgG4 (5D9-γ4). The same blood-sampling schedule was performed as in previous study and animals were euthanized on day 7 or 8 for complete necropsy and pathological examination. PBMC were also obtained.

General well-being was recorded daily, body weight and temperature recorded each time handling of the animals was performed. All clinical chemistry analysis were performed on a COBAS INTEGRA-400+ (Roche, Almere, The Netherlands), and measurements included: Sodium, potassium, chloride, calcium, carbon dioxide, phosphate, alkaline phosphatase, bilirubine total, gamma GT, ASAT (SGOT), ALAT (SGTP), LDH, cholesterol, total protein, albumine, glucose, creatinine, urea, creatine kinase (CKL). All haematology analysis were performed on a Sysmex XT-2000i and measurements included: total white cell count and differential, red cell count, reticulocytes, platelets, hematocrit, hemoglobin, mean cell volume. A full pathology examination was performed on the following organs: adrenals, aorta, brain (brain stem, cerebrum and cerebellum), caecum, colon, duodenum, epididymus, heart, ileum, jejunum, kidneys, liver, lungs, lymph nodes, oesophagus, ovaries, pancreas, parotid salivary glands, pituitary, prostate, sciatic nerve, skeletal muscle (thigh), skin (flank), spleen, sternum (with bone marrow), stomach, salivary glands (submax. trachea), urinary bladder, all gross lesions.

### Results

F45D9-γ1 and F45D9-γ4 administration did not result in deviations of the general well being of the animals. There were no post-dosing signs or treatment-related clinical signs during the treatment period. No animals died prematurely. No major deviations in haematology values and clinical chemistry related to treatment were seen.

F45D9-γ1 administration induced abnormal pathological findings in the liver, pancreas, duodenum, spleen, lymph nodes, lung and kidneys. Findings include inflammation of these organs. In the liver of the most severely affected animal, hepatocellular necrosis, apoptosis and degeneration with hemorrhage and bile stasis was found, in the spleen lymphoid apoptosis and necrosis with white pulp hypoplasia was found and in the lymph nodes follicular hyperplasia with lymphoid apoptosis and necrosis was found. In the other two animals treated with F45D9-γ1 the lesions were comparable, but less pronounced.

F45D9-γ4 administration did not induce apoptosis or necrosis and pathological findings were mild.

Based on the results of the previous toxicological study, where the human anti-Fas antibody F45D9-γ4 mAb showed no toxicity in marmosets after a 5 mg/kg single dose, another toxicity study was conducted on male common marmosets using repeated doses of F45D9-γ4 for a 4 week period. Male common marmosets (Callithrix jacchus; Huntingdon Life Science, Huntingdon, UK) were administered intravenously (bolus) with F45D9-γ4 mAb at a dose of 1, 5 or 5 or 15 mg/kg/occasion once every 7 days for 4 weeks, namely, for 4 times in total (dosed on days 1, 8, 15 and 22). The treatment was conducted in three groups (one group per does) and 3 animals were allocated in each group.

General well-being and body weight were recorded twice weekly. Clinical chemistry and haematology analysis were performed at pretreatment and at termination. On day 29 the animals were euthanized for complete necropsy and pathological examination.

### Results

F45D9-γ4 repeated doses administration did not result in deviations of the general well being of the animals. There were no post-dosing signs or treatment-related clinical signs during the treatment period. No animals died prematurely. Haematological investigations after 4 weeks of treatment revealed no toxicologically significant differences from the pre-treatment values. Clinical chemistry showed increased liver enzymes (ASAT, ALAT, ALP, as well as bilirubin) in two animals after 4 weeks of treatment with 15 mg/kg F45D9-γ4.

It was concluded that four, once weekly, i.v. (bolus) administration of F45D9-γ4 mAb to male common marmosets was well tolerated at doses up to 15 mg/kg.

After this study a pilot pharmacokinetics and receptor occupancy study was performed using single dose administrations of 1.5 or 5 or 15 or 45 mg/kg of F45D9-γ4 mAb. The study had also as main objective the tolerability to a single 45 mg/kg dose administration. The treatment was conducted in 4 groups (one group per dose) and 2 male animals were allocated in each group. In the 5 mg/kg dose group another 2 female animals were allocated. Animals were euthanized on day 22.

The results of this study showed no post-dosing signs or treatment-related clinical signs during the treatment period. No animals died prematurely. The study demonstrated that single dose administration of 45 mg/kg in marmosets was well tolerated. There were no pathological findings which were considered to be related to treatment with F45D9-γ4.

### EXAMPLE 16

### Effect of the Human F45D9 Anti-Fas Antibody in Skin Explant Model of Human Graft-Versus-Host Disease (GVHD)

The aim of the this study was to investigate the effect of anti-Fas monoclonal antibody F45D9-γ1 or F45D9-γ4 on inhibiting GvH Reaction (GvHR) as well as on cytokine release in Mixed Lymphocyte reaction (MLR) and skin explant supernatants, using an established skin explant model, developed by Professor Anne Dickinson at the University of Newcastle. This skin explants model mimics the GvHD in vitro and predicts GvHD outcome, since significant correlation was shown between GvHR in the skin explants and the clinical GvHD grade (Sviland L. et al, 1990, Bone Marrow Transplant 5:105-109; Wang X. N. et al, 2006, Biol Blood Marrow Transplant 12:152-159).

The skin explants assay was set up in a complete mismatched condition in order to test a clinical GvHD grade III-IV scenario. A “milder” setting by using HLA-matched patient and donor pairs and skin was also tested. This skin explants model is a unique assay to study the second and third phase of the GvHD response, i.e. the primary involvement of patient and donor cells with activation of donor-allospecific T cells, and the effect of activated donor T cells on a target organ of GvHD, i.e. the skin.

### Skin Explant Model

The skin explant model has been described in detail previously (Dickinson A. M. et al, 1988, Bone Marrow Transplant 3:323-329; Sviland L. et al, 1990, Bone Marrow Transplant 5:105-109) and is outlined in the overview flow chart below. The model consists of 3 main steps, including a primary MLR to activate donor-allospecific T cells, a coculture of patient skin with activated donor T cells to induce graft-versus-host (GvH)-type tissue damage, and an in situ histopathologic evaluation of the severity of skin tissue damage. Briefly, the MLR was set up in the GvH direction by using patient PBMCs as stimulator cells (20 Gy of irradiation) and an equal number of donor PBMCs as responder cells. At day 5-7 of MLR, standard 4-mm punch skin biopsy samples were obtained from patients. The skin biopsy samples were trimmed of excess dermis, dissected into small sections of equal size, and cocultured with MLR-primed donor responder cells. The skin sections cultured with medium alone were used as background controls. After 3 days of coculture, skin sections were fixed in 10% buffered formalin and stained with hematoxylin and eosin. The histopathologic evaluation of the skin sections was performed blindly by 2 observers and confirmed by an independent histopathologist. On the basis of the severity of histopathologic changes, skin GvHR was defined as grades I to IV according to the Lerner grading system (Lerner, K. G. et al, 1974 Transplant Proc, 6:367-371). All background controls displayed a skin GvHR of grade I or less. A skin GvHR of grade 0 or I was considered negative, and a skin GvHR of grade II or higher was considered as positive. In the case of the HLA-mismatched situation, the MLR was set up by using PBMCs from patients who underwent autologous Haematopoietic Stem Cell Transplantation (HSCT) or plastic surgery as stimulators and PBMCs from an unrelated healthy blood donor as responders. The MLR-primed cells were then cocultured with skin sections taken from the corresponding stimulator.

### Overview of the Skin Explants Assay

MLR—The MLR was set-up either in the HLA mismatched setting or by using HLA matched patient and donor cells. The F45D9 or control antibody was either added at A in the MLR alone (not then added to skin—one experiment) or at B (after MLR at the time of addition of responder cells to skin) or at A+B (at the initiation of the MLR and at the time of addition of responder cells to skin.

1×10⁷ recipient cells×1×10⁷ normal laboratory donor cells
±Antibodies to be Tested (control or F45D9 antibody)—A
Incubate 7 days
Responder cells at day 7 added to patient skin
4 mm punch biopsy from patient
B—Co-incubate 3 days±antibodies to be tested (control or F45D9 antibody)
Routine histopathology
GvHR readout—Grades I-IV

The antibodies used to test the effect on GvHR were F45D9-γ1 (concentration tested were: 0.15; 1.5; 5 and 15 ug/ml), or F45D9-γ4 (concentration tested were: 1.5 and 15 ug/ml) or their respective negative control antibodies: Human IgG1 kappa (from Sigma, Cat 15154) or Human IgG4 kappa (from Sigma, Cat I4639). The F45D9 or control antibody was either added at the initiation of the MLR (not then added to skin) or after MLR at the time of addition of responder cells to skin, or at both stages. The effect of F45D9-γ4 mAb in GvHR was studied only in the HLA-mismatched setting.

### Results

From a total amount of 30 experiments carried out under both HLA mismatched and matched conditions, 26 experiments were showing positive GvHR (17 of 21 in F45D9-γ1 study and 9 of 9 in F45D9-γ4 study. The results on the effect of F45D9-γ1 or F45D9-γ4 mAb antibody are based on those experiments.

Results from the study provide indication of the following.

F45D9-γ1 appeared to down-regulate GvHR in HLA-matched and HLA-mismatched responder induced skin GvHR at the primary and/or secondary stage of the reaction (at the start of MLR and after MLR when activated cells are cultured with skin, respectively) in 8/17 positively evaluated samples, representing a 47% positive effect.

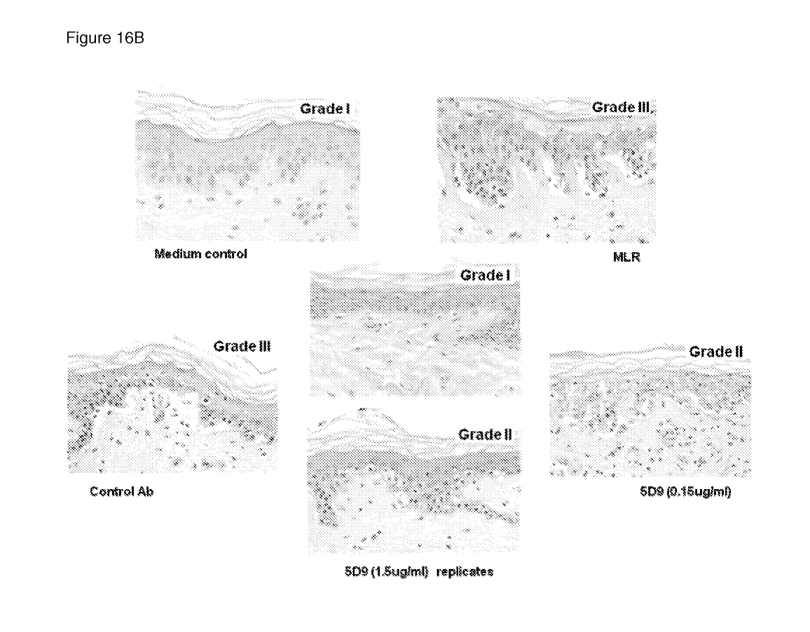

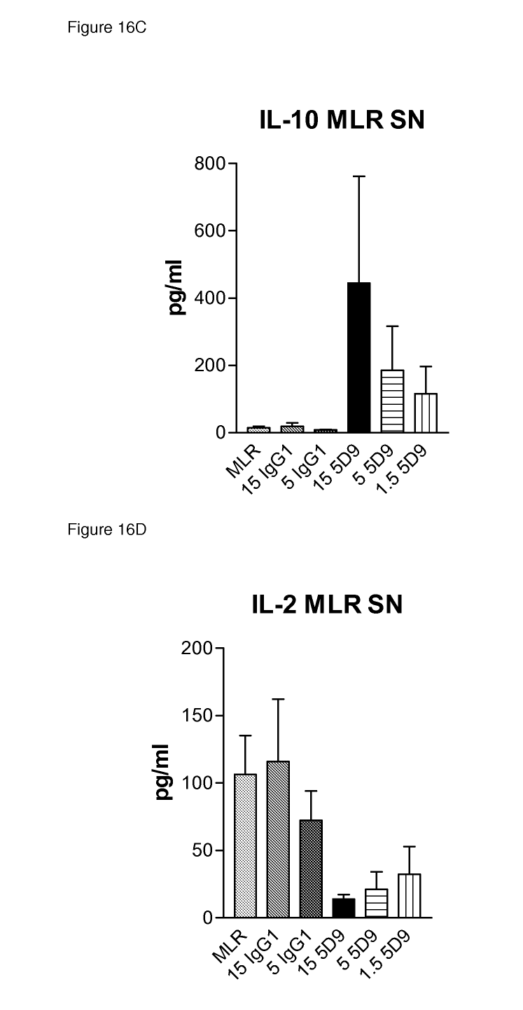

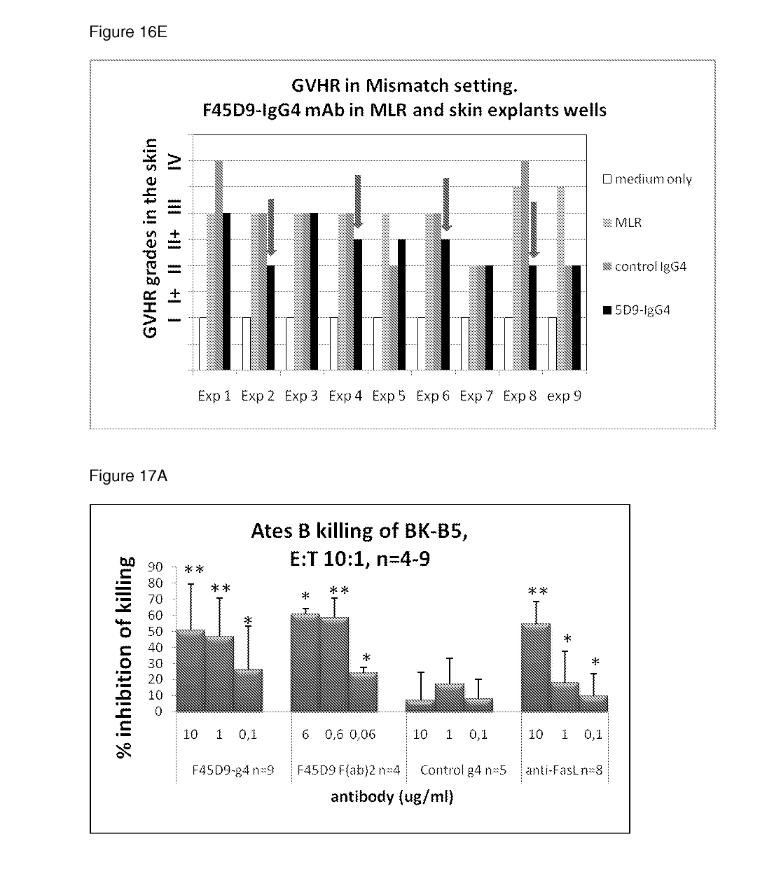

In the HLA-mismatched setting with antibodies added to both MLR and skin explants or to skin explants only, F45D9-γ1 appeared to down-regulate GvHR in 7/14 positively evaluated samples representing 50% positive effect. FIG. 16A illustrates F45D9-γ1 effect down-regulating GvHR in three out of 6 experiments with HLA-mismatched setting adding antibodies to MLR and skin explants wells. FIG. 16B illustrates F45D9-γ1 effect down-regulating GvHR from III to II at 0.15 ug/ml and to I at 1.5 ug/ml in a mismatched setting adding antibodies on skin explants only.

The presence of F45D9-γ1 in MLR or and in skin supernatants, respectively tended to up-regulate IL-10 production in a dose dependent manner and to down-regulate I1-2 production. Results from MLR supernatants are shown in FIG. 16C and FIG. 16D.

F45D9-γ4, added to both MLR and skin explants in HLA-mismatched setting, appeared to down-regulate GvHR in 4/9 positively evaluated samples representing 44% positive effect. Concentration dependence was indicated by a stronger effect at 15 ug/ml as compared to 1.5 ug/mL of F45D9-γ4. FIG. 16E illustrates F45D9-γ4 effect down-regulating GvHR in four out of 9 experiments with HLA-mismatched setting adding antibodies to MLR and skin explants wells F45D9-γ4 was only slightly blocking GvHR in 1/9 experiments when the antibody was only added to skin explants.

F45D9 mAb has been shown to inhibit GvHR in the clinically predictive explant assay. The experiments indicate that F45D9 may be used, optionally together with other prophylaxis regimens, to prevent GvHD (i.e. as shown by adding the antibody at the start of the MLR) and also therapeutically to reduce GvHD (as shown by adding the antibody after the MLR when the cells are activated).

GvHD involves pathological damage caused by donor T cells and the subsequent damage by cytokines. IL-2 is one of the main initial cytokines produced by T cells which aids in proliferation and expansion; IL-10 down regulates the effect of IL-2, INFα and IFNγ. The cytokine modulation induced by F45D9 as shown in our results, specially the down-regulation of IL-2 and up-regulation of IL-10, may be how F45D9 antibody reduces the pathological damage in GvHD.

### EXAMPLE 17

### Effect of F45D9-γ4 mAb on Human Cytotoxic T Cell (CTL) Activity In Vitro

One of the main mechanisms involved in the effector phase of acute GVHD is the direct cell mediated cytotoxicity of host target cells by activated donor T cells (CTL). Fas/FasL is one of the cytolytic mechanisms used by these T cells (and other cytolytic cells) to kill their targets.

The purpose of these studies is to investigate the dose dependent ability of F45D9-γ4 to block specific T cell cytotoxic activity of target cells. For this purpose we have used allogeneic polyclonal HLA-A2-restricted T cell lines obtained from Dr. Victor Levitsky (from Johns Hopkins School of Medicine, Baltimore, USA) and HLA-typed EBV transformed lymphoblastoid cell lines (LCL) as target cells.

The capacity of the F45D9-γ4 antibody to block killing of target cells by allogeneic CTLs was investigated by means of ⁵¹Cr release from target cells (HLA-A2 positive EBV EBNA-4-expressing lymphoblastoid cell line BK-B5) after 16 hours incubation of effect and target at ratios 10:1 or 5:1. One of the effector cells (Ates B) was derived from a patient with a genetic mutation in the perforin gene, causing a premature stop in transcription and no functional perforin protein. Ates B is thus unable to kill target cells in a perforin dependent manner. Alternatively, concanamycin A (CMA; Sigma, Cat C9705) was used to block perforin mediated cytolysis in an allogenic T clone derived from healthy donor (310905/Mon-B1). F45D9 F(ab′)₂ fragments, anti-FasL mouse monoclonal antibody NOK-2 (BD Bioscience, Cat 556375), and isotype human IgG4 kappa control antibody (Sigma, Cat 14639) were also used in the experiments.

### Results:

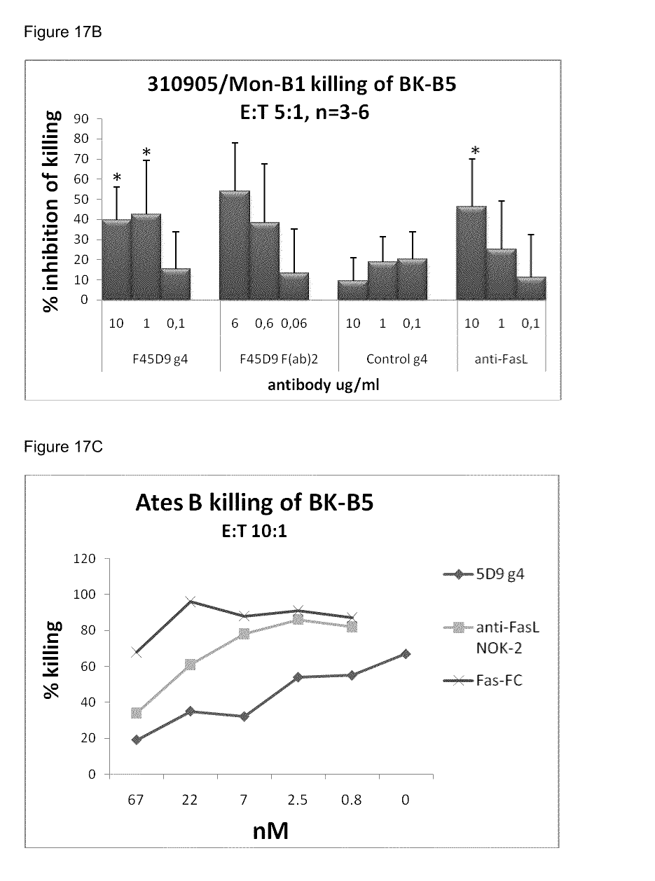

FIG. 17A shows that F45D9-γ4 mAb efficiently blocks Ates B killing of HLA-A2 expressing LCL BK-B5, in a dose dependent manner at a concentration in the range of 0.1 ug/ml to 10 ug/ml. On average 50% of the killing could be blocked at concentration of 10 ug/ml. Similar results were seen with F45D9 F(ab)2 fragments, but not with the isotype control.

FIG. 17B shows that F45D9-γ4 mAb also blocks cytolysis mediated by a CMA treated allogeneic T cell clone from a healthy donor (310905/Mon-B1) of BK-B5 targets, in a dose dependent manner at a concentration in the range of 1 ug/ml to 10 ug/ml. On average 40% of the killing could be blocked at concentration of 10 ug/ml.

F45D9-γ4 mAb was shown to be more efficient in blocking T cell cytolysis as compared to anti-FasL mouse monoclonal antibody NOK-2 (FIGS. 17A and 17C with Ates B and FIG. 17B with 310905/Mon-B1 as effector CTLs).

F45D9-γ4 mAb was also superior when compared on a molar basis to soluble monomeric Fas in blocking Ates B killing of BK-B5 target cells (FIG. 17C).

These results using allogeneic CTLs support the rational for inhibiting FasL/Fas in the effector phase of GVHD with F45D9 mAb.

TABLE 1
Immunization protocols
Dates shown are Date of Immunization (yy-mm-dd) rFas (PreproTech)

| Mouse 18 | | | Mouse 3 | | |
|---|---|---|---|---|---|
| (2^{nd} batch mice) | | | (3^{rd} batch mice) | | |
| Serial | Date | Immunogen | Serial | Date | Immunogen |
| 1^{st} | 03-07-25 | Whole Jurkat cells + | 1^{st} | 03-10-09 | 20 ug rFas + |
| | | 10 ug rFas | | | adjuvant |
| 2^{nd} | 03-08-05 | Whole Jurkat cells + | 2^{nd} | 03-10-21 | 10 ug rFas + |
| | | 10 ug rFas | | | adjuvant |
| 3^{rd} | 03-08-12 | Whole Jurkat cells + | 3^{rd} | 03-10-30 | 10 ug rFas + |
| | | 10 ug rFas | | | 10 ug FP5, 8, 9, 11, 18 + |
| | | | | | adjuvant |
| 4^{th} | 03-08-21 | 5 ug rFas + | 4^{th} | 03-11-06 | 10 ug rFas + |
| | | 10 ug FP5, 8, 9, 11, 18 + | | | adjuvant |
| | | adjuvant | | | |
| 5^{th} | 03-08-28 | 5 ug rFas + | 5^{th} | 03-11-13 | 10 ug rFas + |
| | | 10 ug FP5, 8, 9, 11, 18 + | | | 10 ug FP11, 18 + |
| | | adjuvant | | | adjuvant |
| 6^{th} | 03-10-30 | 10 ug rFas + | — | n.a. | — |
| | | 10 ug FP5, 8, 9, 11, 18 + | | | |
| | | adjuvant | | | |
| 7^{th} | 03-11-13 | 10 ug rFas + | — | n.a. | — |
| | | 10 ug FP11, 18 + | | | |
| | | adjuvant | | | |
| Boost- | 03-12-03 | rFas + | Boost- | 03-12-03 | rFas + |
| 1 | | FP5, 8, 9, 11,18 | 1 | | FP5, 8, 9, 11, 18 |
| Boost- | 03-12-09 | rFas + | Boost- | 03-12-09 | rFas + |
| 2 | | FP5, 8, 9, 11, 18 | 2 | | FP5, 8, 9, 11, 18 |
| Boost- | 03-12-12 | rFas + | Boost- | 03-12-12 | rFas + |
| 3 | | FP5, 8, 9, 11, 18 | 3 | | FP5, 8, 9, 11, 18 |

TABLE 2
Reactivity of F45D9 on non-human primate cells

| | Non-human primate | | | | | | | | |
|---|---|---|---|---|---|---|---|---|---|
| | | | | Cynomolgus | | Olive | | | |
| | Rhesus macaque | | | macaque | | baboon | Chimpanzee | Marmoset | |
| | B cell | | Activated | B cell | | B cell | B cell | B cell | |
| Antibody | line | PBL | T cell | line | PBL | line | line | line | PBMC |
| Mouse anti- | + | + | + | + | + | + | + | + | + |
| human CD95 | | | | | | | | | |
| (Clone DX2) | | | | | | | | | |
| F45D9 | − | − | − | − | − | − | + | + | + |

1. A binding member that binds human Fas and which comprises an antibody VH domain and an antibody VL domain, the antibody VH domain comprising a VH CDR1, VH CDR2 and a VH CDR3 and the VL domain comprising a VL CDR1, VL CDR2 and a VL CDR3, wherein the VH CDR3 is the VH CDR3 of SEQ ID NO. 7 and optionally wherein the VH CDR1 is the VH CDR1 of SEQ ID NO. 5 and/or the VH CDR2 is the VH CDR2 of SEQ ID NO. 6.

2. A binding member according to claim 1 wherein the VH domain comprises the VH CDR1 of SEQ ID NO. 5, the VH CDR2 of SEQ ID NO. 6 and the VH CDR3 of SEQ ID NO. 7.

3. A binding member according to claim 1 comprising the VH domain of SEQ ID NO. 2.

4. A binding member according to claim 1 wherein the VL domain comprises the VL CDR1 of SEQ ID NO. 8, the VL CDR2 of SEQ ID NO. 9 and the VL CDR3 of SEQ ID NO. 10.

5. A binding member according to claim 1 comprising the VL domain of SEQ ID NO. 4.

6. A binding member according to claim 3 comprising the VL domain of SEQ ID NO. 4.

7. A binding member according to claim 1 that binds human Fas with affinity equal to or better than the affinity of a human Fas antigen-binding site formed by the VH domain of SEQ ID NO. 2 and the VL domain of SEQ ID NO. 4, the affinity of the binding member and the affinity of the antigen-binding site being as determined under the same conditions.

8. A binding member according to claim 1 that inhibits human Fas-mediated apoptosis.

9. A binding member according to claim 8 that inhibits human Fas-mediated apoptosis with a potency equal to or better than the potency of a Fas antigen-binding site formed by the VH domain of SEQ ID NO. 2 and the VL domain of SEQ ID NO. 4, the potency of the binding member and the potency of the antigen-binding site being as determined under the same conditions.

10. A binding member according to claim 1 that mediates a co-stimulatory signal with an anti-CD3 antibody in the proliferation of human T cells.

11. A binding member according to claim 1 that does not induce complement dependent cytotoxicity.

12. A binding member according to claim 1 that inhibits the GVHR in skin tissue sections from experimental skin explants model of human GVHD.

13. A binding member according to claim 1 that does not induce hepatotoxicity in primary human hepatocytes.

14. A binding member according to claim 1 that comprises an scFv antibody molecule.

15. A binding member according to claim 1 that comprises an antibody constant region.

16. A binding member according to claim 15 wherein the antibody constant region is of IgG4 isotype.

17. A binding member according to claim 16 wherein the antibody constant region of IgG4 isotype has mutation S228P.

18. A binding member according to claim 17 that comprises a whole antibody.

19. A binding member according to claim 6 that comprises an antibody constant region.

20. A binding member according to claim 19 wherein the antibody constant region is of IgG4 isotype.

21. A binding member according to claim 20 wherein the antibody constant region of IgG4 isotype has mutation S228P.

22. A binding member according to claim 21 that comprises a whole antibody.

23. An isolated nucleic acid which comprises a nucleotide sequence encoding a binding member or antibody VH or VL domain of a binding member according to claim 6.

24. A host cell transformed with nucleic acid according to claim 23.

25. A method of producing a binding member or antibody VH or VL domain, the method comprising culturing host cells according to claim 24 under conditions for production of said binding member or antibody VH or VL domain.

26. A method according to claim 25 further comprising isolating and/or purifying said binding member or antibody VH or VL variable domain.

27. A method according to claim 26 further comprising formulating the binding member or antibody VH or VL variable domain into a composition including at least one additional component.

28. A method of obtaining a binding member that binds human Fas, the method comprising
providing by way of addition, deletion, substitution or insertion of one or more amino acids in the amino acid sequence of the VH domain of SEQ ID NO. 2 one or more VH domains each of which is an amino acid sequence variant of the VH domain of SEQ ID NO. 2, optionally combining one or more VH domain amino acid sequence variants thus provided with one or more VL domains to provide one or more VH/VL combinations; and/or
providing by way of addition, deletion, substitution or insertion of one or more amino acids in the amino acid sequence of the VL domain of SEQ ID NO. 4 a VL domain which is an amino acid sequence variant of the VL domain of SEQ ID NO. 4, and combining one or more VL domain amino acid sequence variants thus provided with one or more VH domains to provide one or more VH/VL domain combinations;
and
testing the VH domain amino acid sequence variants or VH/VL combination or combinations for to identify a binding member that binds human Fas.

29. A method of obtaining a binding member that binds human Fas, which method comprises:
providing starting nucleic acids encoding one or more VH domains which either comprise a CDR3 to be replaced or lack a CDR3 encoding region, and combining said starting nucleic acid with a donor nucleic acid encoding the VH CDR3 amino acid sequence of SEQ ID NO. 7 such that said donor nucleic acid is inserted into the CDR3 region in the starting nucleic acid, so as to provide product nucleic acids encoding VH domains; or
providing starting nucleic acids encoding one or more VL domains which either comprise a CDR3 to be replaced or lack a CDR3 encoding region, and combining said starting nucleic acid with a donor nucleic acid encoding the VL CDR3 amino acid sequence of SEQ ID NO. 10 such that said donor nucleic acid is inserted into the CDR3 region in the starting nucleic acid, so as to provide product nucleic acids encoding VL domains;
expressing the nucleic acids of said product nucleic acids encoding VH domains and optionally combining the VH domains thus produced with one or more VL domains to provide VH/VL combinations, and/or expressing the nucleic acids of said product nucleic acids encoding VL domains and combining the VL domains thus produced with one or more VH domains to provide VH/VL combinations;
selecting a binding member comprising a VH domain or a VH/VL combination that binds human Fas; and
recovering said binding member that binds human Fas and/or nucleic acid encoding the binding member that binds human Fas.

30. A method according to claim 28, further comprising testing the binding member that binds human Fas for ability to inhibit human Fas-mediated apoptosis.

31. A method according to claim 30 wherein a binding member that binds human Fas and inhibits human Fas-mediated apoptosis is obtained.

32. A method according to claim 31 wherein the binding member that binds human Fas is an antibody fragment comprising a VH domain and a VL domain.

33. A method according to claim 32 wherein the antibody fragment is an scFv antibody molecule.

34. A method according to claim 32 wherein the antibody fragment is an Fab antibody molecule.

35. A method according to claim 31 further comprising providing the VH domain and/or the VL domain of the antibody fragment in a whole antibody.

36. A method according to claim 35 further comprising formulating the binding member that binds human Fas or an antibody VH or VL variable domain of the binding member that binds human Fas into a composition including at least one additional component.

37. A method according to claim 35 further comprising binding a binding member that binds human Fas to Fas or a fragment of Fas.

38. A method comprising binding a binding member that binds human Fas according to claim 6 to Fas or a fragment of Fas.

39. A method according to claim 38 wherein said binding takes place in vitro.

40. A method according to claim 38 comprising determining the amount of binding of binding member to Fas or a fragment of Fas.

41. A method of treatment of a disease, disorder or patient selected from the group consisting of (1) GVHD; (2) HIV-infected individuals, e.g. non-treated HIV-infected individuals with decreasing CD4 T cells and low viral load or anti-viral-treated HIV-infected individuals with controlled viral load but not recovered CD4 T cell counts; (3) Stevens-Johnson syndrome (SJS) or Toxic epidermal necrolysis (TEN); (4) Islet transplantation as treatment for insulin-dependent diabetes (autoimmune diabetes); (5) diseases based on ischemia or ischemic reperfusion injury, e.g. disease based on ischemic reperfusion injury in heart, kidney, liver, lung, gut or brain, such as stroke; diseases based on ischemic reperfusion injury associated with surgery or transplantation; ischemic reperfusion injury associated with thrombolytic therapy or angioplasty; (6) heart disease, ischemic heart diseases, myocardial infarction, heart failure, ischemic reperfusion injury; (7) renal disease, renal failure; renal ischemia; ischemic reperfusion injury, acute renal failure; (8) neurological disorders and injuries, cerebral or spinal cord injury, stroke; and (9) lymphocyte depletion in cancer patients associated to cytotoxic antineoplastic therapy, the method comprising administering a binding member according to claim 6 to a patient with the disease or disorder or at risk of developing the disease or disorder.
